(19) United States
(12) Patent Application Publication
(10) Pub. No.: US 2024/0033403 A1
(43) Pub. Date: Feb. 1, 2024

(54) METHODS OF ALTERING PROTEIN DEPOSITION ON URINARY CATHETERS AND DEVICES

(71) Applicants: University of Maine System Board of Trustees, Orono, ME (US); University of Notre Dame du Lac, South Bend, IN (US)

(72) Inventors: Caitlin Lake Howell, Veazie, ME (US); Marissa J. Andersen, South Bend, IN (US); Ana Lidia Flores Mireles, South Bend, IN (US)

(21) Appl. No.: 18/224,842

(22) Filed: Jul. 21, 2023

Related U.S. Application Data
(60) Provisional application No. 63/458,266, filed on Apr. 10, 2023.
(60) Provisional application No. 63/393,169, filed on Jul. 28, 2022.

(51) Int. Cl. A61L 29/08; A61L 29/06

(52) U.S. Cl. CPC A61L 29/085; A61L 29/06; A61L 2300/424; A61L 2300/404

(57) ABSTRACT

Presented herein are devices, systems, and methods related to liquid infused substrates for use in medical applications. Adhesion of proteins, pathogens, and other substances to medical devices presents an issue. Proteins from the surrounding environment adhere to medical devices, which may, under certain conditions, result in the adhesion of pathogens to the medical device. The presence of these pathogens may result in infections when a medical device is inserted or otherwise placed in vivo (in whole or in part), which may require the removal of the device and/or treatment of the subject with antibiotics. Changing the surface properties of such devices can alter which proteins, pathogens, and/or other substances adhere and/or adsorb to the surface. Accordingly, in some embodiments, the present disclosure provides for technologies for altering surface adhesion and/or absorption of proteins, pathogens and/or other substances by infusing/impregnating a substrate of the device with an impregnation fluid.

### CROSS-REFERENCE TO RELATED APPLICATIONS

This application claims the benefit of and priority to U.S. Provisional Application 63/458,266 filed on Apr. 10, 2023 and U.S. Provisional Application 63/393,169 filed on Jul. 28, 2022, the contents of which are hereby incorporated by reference herein in its entirety.

### STATEMENT REGARDING FEDERALLY SPONSORED RESEARCH

This invention was made with government support under grant number RO1-DK128805 awarded by the National Institutes of Health, CBET-2029378, and CBET-1939710 awarded by the National Science Foundation. The government has certain rights in the invention.

### TECHNICAL FIELD

The present disclosure relates generally to protein deposition on devices (e.g., medical devices, e.g., catheters). In certain embodiments, the disclosure relates to methods for altering protein deposition on urinary catheters.

### BACKGROUND

The global urinary catheters market size was valued at USD 4.65 billion in 2020 and is expected to grow at a compound annual growth rate of 7.0% from 2021 to 2028. The key factors for the urinary catheter market growth is the increase in the number of patients suffering from Urinary Tract Infections (UTIs), urethra blockages, the rise cases of tumors in the urinary tract or reproductive organs, and the rapid growth of the geriatric population.

Catheterization is an exceedingly common procedure in healthcare facilities, with an estimated ˜30 million urinary catheters used in the US and Europe annually. Urinary catheters are used to drain patient's bladders during surgical sedation and recovery in addition to being used in treatment for a variety of conditions. Despite the benefits urinary catheters provide for patients, catheterization causes adverse effects including infections and bladder stones (Andersen & Flores-Mireles, 2019; Feneley, Hopley, & Wells, 2015). The most common complication is catheter-associated urinary tract infection (CAUTI), which accounts for 40% of all hospital acquired infections (HAIs) (Andersen & Flores-Mireles, 2019; Feneley et al., 2015). Catheter placement alone predisposes the patient to CAUTIs, and the risk of developing an infection is directly correlated to the catheter's dwell time. CAUTIs often lead to bloodstream infections and systemic dissemination with a 30% mortality rate, causing significant financial burdens for hospitals and patients (Andersen & Flores-Mireles, 2019; Feneley et al., 2015).

Accordingly, there is a need for improved catheters.

### SUMMARY

Presented herein are devices, systems, and methods related to liquid infused substrates for use in medical applications. Adhesion of proteins, pathogens, and other substances to medical devices presents an issue in the field. Proteins from the surrounding environment adhere to medical devices, which may, under certain conditions, result in the adhesion of pathogens to the medical device. The presence of these pathogens may result in infections when a medical device is inserted or otherwise placed in vivo (in whole or in part), which may require the removal of the device and/or treatment of the subject with antibiotics. Changing the surface properties of such devices can alter which proteins, pathogens, and/or other substances adhere and/or adsorb to the surface. Accordingly, in some embodiments, the present disclosure provides for technologies (e.g., devices, systems, and/or methods(s)) for altering surface adhesion and/or absorption of proteins, pathogens and/or other substances by infusing/impregnating a substrate of the device with an impregnation fluid.

Infusing a substrate (e.g., a polymeric substrate) of a device with an impregnation fluid alters surface properties of the substrate. In certain embodiments, adhesion and/or absorption of proteins to a substrate infused with an impregnation fluid is altered from one that has not been infused with an impregnation fluid. In certain embodiments, a substrate of a device is infused with an impregnation fluid such that an impregnation fluid does not form an overlayer on the substrate. A lack of an overlayer of impregnation fluid (e.g., silicone oil) on a surface is important for medical applications, where release of an impregnation fluid into an organism can result in production of protein aggregates which can cause inflammation or other types of damage within a living system. Previously, it had been understood that a free overlayer of impregnation fluid must be present for a material to resist adhesion by proteins and microorganisms. An anti-adhesion effect can also be achieved without the presence of such a layer. For example, and without wishing to be bound to any particular theory, impregnating a silicone substrate with a silicone oil results in altered adhesion properties of the surface of the substrate by altering the local charges (e.g., gradients of charges) on the substrate's surface. In some embodiments, an impregnated substrate allows proteins with less local surface charge (e.g., weak gradients of charges) to adhere to the substrate's surface, while proteins with more local surface charges (e.g., strong gradients of charges) are unable to adhere, or have reduced ability to interact directly with the substrate. In contrast, in some embodiments, an overlayer of impregnation fluid would prevent adhesion of substantially all proteins to the surface, including proteins with less local surface charge. Locally charged proteins, such as fibrinogen, are in part responsible for the adhesion of pathogens such as uropathogens to the surface of devices, which can result in infections.

In some embodiments, the substrate is infused with an impregnation fluid such that no overlayer or immobilized layer of impregnation fluid is formed on the surface of the substrate.

In one aspect, the disclosure encompasses methods of modifying a polymeric substrate of a medical device, the method comprising: infusing the polymeric substrate with an impregnation fluid such that the polymeric substrate is impregnated with the fluid.

In some embodiments, a polymeric substrate is biocompatible.

In some embodiments, a polymeric substrate comprises silicone.

In some embodiments, a silicone comprises polydimethylsiloxane (PDMS) (e.g., cross-linked PDMS).

In some embodiments, a polymeric substrate comprises a hydrogel, poly(acrylic acid), poly(vinyl alcohol), poly(vinylpyrrolidone), poly(ethylene glycol), polyacrylamide, or a polysaccharide hydrogel.

In some embodiments, an impregnation fluid comprises a hydrophilic liquid (e.g., a liquid comprising ammonia, alcohol(s), one or more amides [e.g., urea], carboxylic acid(s) [e.g., acetic acid]) or a hydrophilic ionic liquid.

In some embodiments, a polymeric substrate comprises an organogel.

In some embodiments, an organogel comprises one or more of the following materials: anthracene, anthraquinone and steroid-based molecules.

In some embodiments, an impregnation fluid is or comprises silicone oil. In some embodiments, the silicone oil is medical grade (e.g., biocompatible). In some embodiments, the viscosity of the silicone oil is from 0.65 cSt to 10,000 cSt (e.g., from 10 cSt to 50 cSt)(e.g., using a viscometer). In some embodiments, a silicone oil comprises trimethoxy-terminated polydimethylsiloxane. In some embodiments, a silicone oil comprises repeating siloxane units and end-blocking siloxane units.

In some embodiments, an impregnation fluid comprises one or more of the following low-volatility polydimethylsiloxanes, cyclic polydimethylsiloxanes (e,g, cyclomethicones), silicone emulsions, silicone fluid blends, thermal silicone fluids, alkyl silicones (e.g., alkyl-methilsiloxane fluids), aryl-alkyl silicones, fluorosilicone fluids, hydrophilic silicones (e.g. polyalkylene oxide silicones), polar silicones, ampliphilic silicones, low-temperature fluids (e.g. polydiethysiloxanes, silahydrocarbons, di/trisiloxane fluids), naturally derived silicones (e.g., MonoAnisyl-terminated polydimethylsiloxane, limonenyl trisiloxane).

In some embodiments, provided methods comprise removing substantially all impregnation fluids from a surface the polymeric substrate (e.g., prior to implantation or insertion into a subject). In some embodiments, provided methods comprise removing substantially all free the silicone oil from a surface the polymeric substrate.

In some embodiments, provided methods comprise mechanically or chemically removing post-infusion excess silicone oil from the surface of the polymeric substrate.

In some embodiments, provided methods do not produce an immobilized liquid layer of silicone on the surface of the polymeric substrate.

In some embodiments, provided methods comprise infusing a polymeric substrate with a silicone oil comprises impregnating the polymeric substrate less than 100% of the maximum absorption capacity (e.g., Qₘₐₓ) of the polymeric substrate.

In some embodiments, infusing a polymeric substrate with a silicone oil comprises impregnating the polymeric substrate from 1% to 99.99% (e.g., from 50% to 99.99%, e.g., from 90% to 95%) of the maximum absorption capacity (e.g., Qₘₐₓ) of the polymeric substrate.

In some embodiments, infusing a polymeric substrate with a silicone oil comprises impregnating the polymeric substrate more than 1% (e.g., more than 50%) of the maximum absorption capacity (e.g., Qₘₐₓ) of the polymeric substrate.

In some embodiments, infusing a polymeric substrate with a silicone oil comprises immersing the polymeric substrate in the silicone oil for a period of time (e.g., at least 1 hour, etc.) (e.g., based at least on one or more physical properties (e.g., thickness, etc.) of the substrate)(e.g., based on the temperature of the silicone oil).

In some embodiments, provided methods reduce adhesion and/or adsorption of one or more protein(s) to a surface of the polymeric substrate. In some embodiments, the one or more protein(s) comprise fibrinogen or serum albumin.

In some embodiments, provided methods increase adhesion and/or adsorption of one or more protein(s) to the polymeric substrate. In some embodiments, the one or more protein(s) comprises one or more members of the group consisting of: UDP-glucose 6-dehydrogenase (UGDH), filamin B (FLNB), and Proteasome 20S Subunit Beta 5 (PSMB5). In some embodiments, the one or more protein(s) are characterized in that the one or more protein(s) are not involved in an immune response of a subject (e.g., a human subject).

In some embodiments, a polymeric substrate comprises an outward facing surface which interfaces with a tissue.

In some embodiments, a polymeric substrate comprises an inward facing surface which interfaces with a biological fluid.

In another aspect, the present disclosure encompasses urinary catheters comprising a polymeric substrate manufactured according to the methods described herein.

In another aspect, the present disclosure encompasses methods of treating a subject using a urinary catheter comprising a polymeric substrate manufactured according to methods described herein.

In another aspect, the present disclosure encompasses methods of modifying a urinary catheter to alter protein adhesion to a polymeric substrate of the catheter, the method comprising: infusing, by immersion for a period of time, a polymeric substrate of the urinary catheter with a silicone oil, wherein the polymeric substrate comprises silicone, wherein the period of time is characterized in that the polymeric substrate is impregnated with the silicone oil; and removing (e.g., stripping) substantially all of the silicone oil from the surface(s) of the catheter (e.g., an overlayer of silicone oil) such that the surface(s) of the polymeric substrate substantially does not comprise free silicone oil.

In another aspect, the present disclosure encompasses methods of modifying a urinary catheter to alter protein adhesion to a polymeric substrate of the catheter, the method comprising: infusing, by immersion for a period of time, the polymeric substrate of the urinary catheter with a silicone oil, wherein the polymeric substrate comprises silicone, wherein the period of time is characterized in that the polymeric substrate is partially impregnated with the silicone oil such that the surface of the substrate substantially does not comprise free silicone oil.

In another aspect, the present disclosure encompasses medical devices comprising: a polymeric substrate, wherein the polymeric substrate is impregnated with a fluid.

In some embodiments, a medical device is an indwelling medical device.

In some embodiments, a medical device is a urinary catheter.

In some embodiments, a polymeric substrate is biocompatible.

In some embodiments, a polymeric substrate comprises silicone. In some embodiments, the silicone comprises polydimethylsiloxane (PDMS) (e.g., cross-linked PDMS).

In some embodiments, a polymeric substrate comprises a hydrogel, poly(acrylic acid), poly(vinyl alcohol), poly(vinylpyrrolidone), poly(ethylene glycol), polyacrylamide, or a polysaccharide hydrogel.

In some embodiments, an impregnation fluid comprises a hydrophilic liquid (e.g., a liquid comprising ammonia, alcohol(s), one or more amides [e.g., urea], carboxylic acid(s) [e.g., acetic acid]) or a hydrophilic ionic liquid.

In some embodiments, a polymeric substrate comprises an organogel. In some embodiments, the organogel comprises one or more of the following materials: anthracene, anthraquinone and steroid-based molecules.

In some embodiments, an impregnation fluid comprises silicone oil. In some embodiments, the silicone oil is medical grade (e.g., biocompatible). In some embodiments, the viscosity of the silicone oil is from about 0.65 cSt to about 10,000 cSt (e.g., from about 10 cSt to about 50 cSt). In some embodiments, the silicone oil comprises trimethoxy-terminated polydimethylsiloxane. In some embodiments, the silicone oil comprises repeating siloxane units and end-blocking siloxane units.

In some embodiments, an impregnation fluid comprises one or more of the following: low-volatility polydimethylsiloxanes, cyclic polydimethylsiloxanes (e,g, cyclomethicones), silicone emulsions, silicone fluid blends, thermal silicone fluids, alkyl silicones (e.g., alkyl-methilsiloxane fluids), aryl-alkyl silicones, fluorosilicone fluids, hydrophilic silicones (e.g. polyalkylene oxide silicones), polar silicones, amphiphilic silicones, low-temperature fluids (e.g. polydiethysiloxanes, silahydrocarbons, di/trisiloxane fluids), naturally derived silicones (e.g., MonoAnisyl-terminated polydimethylsiloxane or limonenyl trisiloxane).

In some embodiments, a polymeric substrate does not comprise a layer of impregnation fluid on the surface of the polymeric substrate. In some embodiments, the polymeric substrate does not comprise an immobilized liquid layer of silicone oil on the surface of the polymeric substrate.

In some embodiments, a silicone oil is impregnated in the substrate at less than 100% of the maximum absorption capacity (e.g., Qₘₐₓ) of the polymeric substrate.

In some embodiments, a silicone oil is impregnated in the substrate from about 1% to about 99.9% of the maximum absorption capacity (e.g., Qₘₐₓ) of the polymeric substrate.

In some embodiments, a silicone oil is impregnated in the substrate at more than about 1% (e.g., about 50%) of the maximum absorption capacity (e.g., Qₘₐₓ) of the substrate.

In some embodiments, the polymeric substrate has reduced adhesion and/or adsorption for one or more protein(s). In some embodiments, the one or more protein(s) comprise fibrinogen or serum albumin.

In some embodiments, the polymeric substrate has increased adhesion and/or adsorption for one or more protein(s). In some embodiments, the one or more protein(s) comprise one or more members of the group consisting of: UDP-glucose 6-dehydrogenase (UGDH), filamin B (FLNB), and Proteasome 20S Subunit Beta 5 (PSMB5).

### BRIEF DESCRIPTION OF THE FIGURES

Drawings are presented herein for illustration purposes, not for limitation. The foregoing and other objects, aspects, features, and advantages of the disclosure will become more apparent and may be better understood by referring to the following description taken in conjunction with the accompanying drawings.

FIG. 1 is an illustrative embodiment of surface protein adhesion to substrates. FIG. 1, panel A is an illustrative embodiment of an exemplary partially silicone-oil indwelling urinary catheter. FIG. 1, panel B is an illustrative embodiment of an exemplary untreated urinary catheter.

FIG. 2 is a series of schematics of the fabrication of different infused silicone samples, according to an illustrative embodiment. “Traditional infusion” refers to samples with a free overlayer of impregnation fluid (e.g., silicone oil). “Partial infusion” and “Overlayer stripping” refer to two methods used to remove a free overlayer of impregnation fluid (e.g., free oil). “Partial infusion” is also described as % Qₘₐₓ, with 100% Qₘₐₓ equivalent to “Overlayer stripping”.

FIG. 3A is a series of immunofluorescent (IF) images of urinary catheters. Urinary catheters are stained with IF antibodies for Fg deposition (Fg; green) and pathogen binding (respective pathogen; red). Yellow in the “MERGE” image is indicative of overlap between Fg and pathogen. Unimplanted catheters were used as controls (C) for autofluorescence, n=3-4.

FIG. 3B is a series of bar graphs quantifying the IF images of FIG. 2A. For all graphs error bars show the standard error of the mean (SEM). Between 3 and 5 replicates of n=4312 each were performed for each pathogen and condition

FIG. 3C is a series of representative images from a single bladder illustrating the interaction of uropathogens (red), Fg (green), and nuclei (blue) on the bladder urothelium (U), and in the lumen (L). Scale bar is 50 μm.

FIG. 4 is a series of IF images corresponding to the IF images of FIG. 2C. Montages of FIG. 1 merged images. Mice were implanted and infected with 1×10⁶ CFU of the respective uropathogens. At 24 hpi, bladders tissues were harvested, fixed, and parafilm-embedded. Bladder were subjected to IF analysis, antibodies staining were used to detect Fg (anti-Fg; green), uropathogens (red), and cell nuclei (blue; DAPI). Scale bars, 50 μm. Magnification 100×.

FIG. 5 is a series of bar graphs showing the relative percent binding pathogens to catheters. Uropathogens were tested for their ability to bind to protein coated (Fg; fibrinogen and BSA; bovine serum albumin) and uncoated (UC) silicone catheters. Each panel represents the respective uropathogen which the pathogen bound to—(A) E. faecalis OG1RF, (B) E. coli UTI89, (C) P. aeruginosa PA01, (D) K. pneumoniae TOP52, (E) A. baumannii UPAB1, and (F) C. albicans SC5314. Each panel the standard error of the mean (SEM) are represented as error bars. Between 3-5 replicates of n=4-12 each were performed for each pathogen and condition. Differences between groups were tested for significance using the Mann-Whitney U test. *P≤**P≤0.01, ***P≤0.001; ****P≤0.0001; ns, difference not significant.

FIG. 6 shows how the relative binding percentage was calculated for FIG. 5, panels A-F. Relative binding percentage was calculated as the sample (e.g., the signal in the blue square) minus the background (e.g., the signal in the purple square) divided by the average of the 0% Qₘₐₓ controls. The resulting value was multiplied by 100 and presented as a percentage.

FIG. 7 is a series of graphs characterizing properties of silicone and Tygon® tubing infused with an impregnation fluid. Panel A shows the weight of silicone tubes measured at designated time points before and during silicone oil infusion. The mean (±SEM) of n=5 silicone tubes over infusion time is shown in this panel. Panel B shows the weight of Tygon® tubes measured at designated time points before and during silicone oil infusion. The mean (±SEM) of n=5 silicone tubes over infusion time is shown in this panel. Panel C shows the weight of mouse catheters measured at designated time points before and during silicone oil infusion. The mean (±SEM) of n=5 mouse catheter over infusion time is shown in this panel. Panel D shows kinetics of silicone oil infusion on silicone and Tygon® tubes. Panel E shows kinetics of silicone oil infusion on mouse silicone catheters. Panel F shows the change in length, outer diameter, and inner diameter of silicone catheters (n=5) before and after infusion. Panel G shows change in the length, outer diameter, and inner diameter of mouse catheters (n=5-10) measured before and after infusion.

FIG. 8A is a series of IF images of urinary catheters visualizing deposition of fibrinogen (Fg; green) on UM (unmodified) and LI (liquid infused) substrate (LIS) catheter material. Ctl is representative of control catheters that were not implanted.

FIG. 8B is a bar graph showing quantification of deposition of fibrinogen (Fg; green) on UM (unmodified) (black bars) and LI (liquid infused) substrate (LIS) (white bars) catheter material using IF staining. Three replicates with n=2-3 each.

FIG. 8C is a series of bar graphs showing pathogen adhesion to catheters. Each panel is represents the respective uropathogen which the pathogen bound to. The corresponding pathogens are listed as follows for each panel: (A) E. faecalis OG1RF, (B) E. coli UTI89, (C) P. aeruginosa PA01, (D) K. pneumoniae TOP52, (E) A. baumannii UPAB1, and (F) C. albicans SC5314. Each panel the standard error of the mean (SEM) are represented as error bars. The experiment was conducted for 3 replicates with 3 samples for each replicate. The error bars represent SEM.

FIG. 9 is a series of images and graphs depicting colonization of bacteria. Panels A-F of FIG. 7 are graphs depicting bacterial distribution in an organ or on a catheter when using liquid infused silicone (LI) or unmodified (UM) catheters. Panels G-L of FIG. 7 are IF images of urinary catheters demonstrating binding of pathogens (PA) and fibrinogen (Fg) to unmodified (UM) or liquid infused silicone (LI) catheters. Each panel is labeled with the respective pathogen being studied.

FIG. 10 is a series of bar graphs depicting the amount of pathogen localized to fibrinogen (Fg) on an unmodified (UM) or liquid infused silicone (LI) catheter. The corresponding pathogens are listed as follows for each panel: (A) E. faecalis OG1RF, (B) E. coli UTI89, (C) P. aeruginosa PA01, (D) K. pneumoniae TOP52, (E) A. baumannii UPAB1, and (F) C. albicans SC5314. Each panel the standard error of the mean (SEM) are represented as error bars. Quantification of uropathogen-Fg colocalization on UM and LIS-catheters from mice catheterized and infected with one of six uropathogens. Quantification was done using pixel color counter from Fiji where colocalization (yellow) of Fg (green) and pathogen (red) were quantified and compared to the total pathogen colonization of the catheter.

FIG. 11 is a series of images of catheterized mouse bladders. Unmodified (UM) catheters are presented next to liquid infused silicone (LI) catheters. Panels A-F and M of FIG. 8 are hematoxylin and eosin (H&E) stained images. Panels G-L of FIG. 11 are immunofluorescent (IF) images of catheterized mouse bladders.

FIG. 12A is a graph of the total protein abundance in unmodified (UM) catheters and liquid infused (LI) substrate (LIS) catheters. A subset of UM catheters and LIS-catheters taken from mice 24 hpi with E. faecalis were assessed for protein deposition via mass spectrometry 4 UM catheters and 5 LIS-catheters were used. Intensities of the 95% most abundant proteins were summed in a total proteome approach and compared between the UM-catheter and the LIS-catheter groups.

FIG. 12B is a volcano plot of a liquid infused silicone (LI) catheters. A subset of UM catheters and LIS-catheters taken from mice 24 hpi with E. faecalis were assessed for protein deposition via mass spectrometry 4 UM catheters and 5 LIS-catheters were used. A volcano plot was created for a subset of proteins using the mean rank difference and Mann-Whitney statistical analysis to generate p-values. Negative mean rank difference indicates less protein on the LIS catheter than on the UM catheter and a significant difference is shown with a −log 10(P-value) over 1.3. The Fg chains (α-, β-, and γ-) are highlighted in green, serum albumin in orange, UDP-glucose 6-dehydrogenase, filamin-B and proteasome subunit beta type-5 are in yellow.

FIG. 13 is a series of immunofluorescent (IF) images of bladder tissue from mice catheterized with unmodified or liquid infused catheters corresponding to FIG. 4. Mice were implanted with either an unmodified catheter or a liquid-infused catheter and infected with 1×10⁶ CFU of the respective uropathogens. At 24 hpi, bladders tissues were harvested, fixed, and parafilm embedded. Bladder tissues were subjected to IF analysis, antibody staining was used to detect Fg (anti-Fg; green), uropathogens (red), neutrophils (anti-Ly6G; white) and cell nuclei (DAPI; blue). Scale bars are 500 Images are stitched 2×2 tiles at 20× magnification.

FIG. 14 is a drawing of liquid-infused silicone (LIS) and unmodified (UM) catheterized bladders, according to an illustrative embodiment. A liquid-infused silicone (LIS)-catheter reduces bladder inflammation, incidence of catheter-associated urinary tract infection (CAUTI), and dissemination as compared to UM catheters. Urinary catheter-induced inflammation promotes the release of fibrinogen (Fg) into the bladder to heal physical damage. Consequently, this Fg is deposited onto the catheter creating a scaffold for incoming pathogens to bind, establish infection, and promote systemic dissemination. However, catheterization with a LIS-catheter reduces Fg deposition onto its surface; thus, reducing the availability of a binding scaffolds for incoming pathogens. Consequently, overall bladder colonization and systemic dissemination are reduced making LIS-catheters a strong candidate for CAUTI prevention.

FIG. 15A is a series of panels describing fabrication of liquid-infused catheters, according to an illustrative embodiment. A catheter (red) is submerged in a liquid (blue) until equilibrium is reached. The catheter (purple) is then removed from the liquid. Excess liquid on the surface (red) is then stripped away to leave a modified surface.

FIG. 15B is a graph showing the contact angle of non-infused (red), infused (purple, outlined in red), and infused+wiped (purple) silicone tube samples.

FIG. 15C is a graph showing the sliding angle of non-infused (red), infused (purple, outlined in red), and infused+wiped (purple) silicone tube samples.

FIG. 16A shows an illustrative embodiment of the mechanism of action of silicone oil-infusion on commercial silicone catheter surfaces. Commercial silicone catheters have nm-scale heterogeneity of surface charge. Adding a free silicone fluid, which can migrate through the polymer and associate positive charges to negative (and vise-versa), allows the system to become closer to fully neutral.

FIG. 16B shows exemplary proteins with neutral charges and non-neutral charges, which have altered adsorption/adhesion characteristics to silicone oil-infused silicone catheters. Proteins with closer to neutral surface charge (e.g., having weak gradients of charges) show increased adsorption/adhesion to silicone oil-infused silicone catheters. Exemplary more neutrally surface charged proteins include UDP-glucose 6-dehydrogenase (UGDH), Filamin-B (FLNB), and Proteasome subunit beta type-5 (PSMB5). Proteins with more positive or negative surface charge show decreased adsorption/adhesion to silicone oil-infused silicone catheters. Exemplary proteins with localized surface charges include fibrinogen and serum albumin.

FIG. 17, panels A-D show changes of silicone samples under different infusion conditions, according to an illustrative embodiment. % Qₘₐₓ refers to various degrees of partial impregnation. FIG. 17, panel A shows % Qₘₐₓ increases with increasing infusion time, then reaches plateau after 60 hours of infusion. FIG. 17, panel B shows oil uptake by silicone samples increases with increasing infusion time, then plateaus after 60 hours of infusion. FIG. 17, panel C shows the tilt angle of silicone samples infused under different infusion conditions is shown. FIG. 17, panel D shows droplet velocity of silicone samples infused under different infusion conditions. Samples without droplet movement are marked as 100 s in the graph. For all graphs, error bars show the standard deviation (SD), n=3.

FIG. 18, panels A-B show silicone oil loss of silicone samples via repeated exposure of air-water interface. FIG. 18, panel A shows silicone oil loss of fully impregnated silicone samples with or without the free silicone oil overlayer stripped. FIG. 18, panel B shows partially impregnated silicone samples with or without the free silicone oil overlayer stripped. These silicone samples were then tested for silicone oil loss through water dipping. For all graphs, error bars show the standard deviation (SD). Differences between groups were tested for significance using the Welch's t test. **, P≤0.005. n=3.

FIG. 19, panels A-D show fibrinogen and E. faecalis adhesion levels decrease with increasing degrees of impregnation of the substrate. FIG. 19, panel A shows silicone discs were stained with immunofluorescence (IF) for fibrinogen deposition (green). FIG. 19, panel B shows silicone discs were stained with immunofluorescence (IF) for E. faecalis (red) binding. Non-infused silicone discs incubated in PBS were used as controls as controls for autofluorescence. FIG. 19, panel C shows quantification of fibrinogen localization on silicone discs from panel A. FIG. 19, panel D shows quantification of E. faecalis localization on silicone discs from panel B. For all graphs, error bars show the standard deviation (SD). Differences between groups were tested for significance using the Kruskal-Wallis test. *, P<0.05; **, P<0.005; ***, P<0.0005 and ****, P<0.0001. 3 replicates of n=3-4 each were performed for each condition.

FIG. 20, panels A-C show confocal microscopy cross-section images of a PDMS substrate. FIG. 20, panel A shows a cross-section of PDMS. FIG. 20, panel B shows PDMS fully infused with silicone oil. FIG. 20, panel C shows PDMS fully infused with silicone oil after the overlayer was stripped. Overlayer stripping removes any free silicone oil from the surface. The scale bar in the lower left hand corner of each panel is 50 μm in length.

FIG. 21, panels A and B quantify the amount of silicone oil removed from the surface of catheters by passing the catheter through an air-water interface (μL/mm). FIG. 21, panel A shows the amount of silicone oil removed from a non-infused catheter section (“non-infused”), a fully infused LIS-catheter section (T), and a fully infused LIS-catheter section with the silicone oil overlayer stripped (OS). FIG. 21, panel B shows the amount of silicone oil removed from LIS-catheter sections at varying levels of infusion with silicone oil either having the overlayer stripped (dark grey) or left intact (light grey). *P<0.05, ****P<0.0001.

FIG. 22, panels A and B characterize protein and bacterial adhesion on catheter sections. FIG. 22, panel A shows fibrinogen adhesion images (left) and quantification (right) on non-infused (control) PDMS catheters, fully infused PDMS LIS-catheters (traditional infusion, T), and fully infused PDMS LIS-catheters with the overlayer stripped (OS). FIG. 22, panel B shows E. faecalis adhesion images (left) and quantification (right) on non-infused (control) PDMS catheters, fully infused PDMS LIS-catheters (traditional infusion, T), and fully infused PDMS LIS-catheters with the overlayer stripped (OS).

FIG. 23A shows tilt angle analysis of catheter sections.

FIG. 23B shows droplet velocity on catheter sections.

FIG. 23C, panels i and ii show fibrinogen adhesion images (FIG. 23, panel i) and quantification (FIG. 23, panel ii) as the amount of oil in the system as a function in decreasing oil content.

FIG. 23D, panels i and ii show E. faecalis images (FIG. 23D, panel i) and quantification (FIG. 23D, panel ii) as a function of decreasing silicone oil infusion.

FIG. 24, panels A-C show droplet sliding speed for water droplets with either a neutral, positive, or negative charge. FIG. 24, panel A shows the sliding speed of water droplets alone, which have a neutral charge. FIG. 24, panel B shows the sliding speed of crystal violet in water, which has a positive charge. FIG. 24, panel C shows the sliding speed of bromophenol blue in water, which has a negative charge.

FIG. 25, panels A-D show LIS-catheters reduced uropathogenic E. coli catheter-associated UTI and systemic dissemination during prolonged urinary catheterization. FIG. 25, panel A shows results from bladder tissue imaging. FIG. 25, panel B shows results from catheter imaging. FIG. 25, panel C shows results from kidney tissue imaging. FIG. 25, panel D shows results from spleen tissue imaging. All animal studies for CFUs had at least 10 animals per strain and catheter type. Differences between groups were tested for significance using the Mann-Whitney U test. *, P<0.05; **, P<0.005; ***, P<0.0005; and ****, P<0.0001.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C1-C20 for straight chain, C2-C20 for branched chain), and alternatively, about 1-10. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C1-C4 for straight chain lower alkyls).

Aryl: The term “aryl” used alone or as part of a larger moiety as in “aralkyl,” “aralkoxy,” or “aryloxyalkyl,” refers to ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system and exemplary groups include phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Biocompatible: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their use in vivo does not induce significant inflammation or other such adverse effects.

Impregnate: The term “impregnate” as used herein refers to infusing a material with a fluid such that it swells the material. In certain embodiments, a material is partially impregnated with a fluid. In certain embodiments, a material is fully impregnated with a fluid. Exemplary materials and impregnation fluids are described herein. In certain embodiments, the degree of impregnation of a material is measured based on the relative amount of fluid (e.g., an impregnation fluid) the material absorbs or infused with. In certain embodiments, impregnation occurs through diffusion of an impregnation fluid into a substrate.

Absorption Capacity: The term “absorption capacity” is used to describe the amount of an impregnation fluid that can be absorbed by or infused into a substrate. In certain embodiments, the absorption capacity of a material is dependent on the material and/or methods used to impregnate a material with a fluid. For example, the temperature of the substrate and/or impregnation fluid, viscosity of the impregnation fluid, cross-linking density of the substrate, composition of the impregnation fluid, composition of the substrate, period of time of infusion of the impregnation fluid, and other factors (e.g., as disclosed herein) may alter the absorption capacity. In certain embodiments, Qₘₐₓ is a ratio of the difference between the mass of the material when substantially fully infused (M_{swollen}) with an impregnation fluid and the original mass of the material (M_{Original}) to the original mass of the material (M_{Original})

$$( i . e . , {Q}_{max} = \frac{( {M}_{Swollen} - {M}_{Original} )}{( {M}_{Original} )} ) .$$

In certain embodiments, Qₘₐₓ is measured at ambient room temperature (e.g., about 25° C.) and pressure (e.g., about 1013.25 hPa). In certain embodiments, the absorption capacity is expressed as a percentage of Qₘₐₓ where 0% Qₘₐₓ is indicative of the material having not been infused with the impregnation fluid and 100% Qₘₐₓ is indicative of the material having been substantially completely infused with the impregnation fluid.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION

It is contemplated that systems, devices, methods, and processes of the claimed invention encompass variations and adaptations developed using information from the embodiments described herein. Adaptation and/or modification of the systems, devices, methods, and processes described herein may be performed, as contemplated by this description.

Throughout the description, where articles, devices, and systems are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

The technologies presented herein relate to are devices, systems, and methods related to substrates infused with an impregnation fluid for use in medical applications. Deposition of material (e.g., proteins) on a surface of a substrate (e.g., a polymeric substrate) of a medical device occurs when a medical device is implanted into a subject. Infusing a substrate with an impregnation fluid results in alterations to surface properties of the substrate. In certain embodiments, the technologies disclosed herein relate to infusing a substrate with an impregnation fluid such that there is no overlayer of or excess impregnation fluid present on a surface of the substrate. Excess impregnation fluid on a surface could lead to substantially total inhibition of all proteins or materials onto a surface.

New and current management guidelines for CAUTIs have resulted in moderate reductions in incidences. The standard treatment for patients with symptomatic CAUTI is catheter removal and replacement and an antibiotic regiment. However, this approach is not effective because biofilms on the catheter surface protect microbes against antibiotics and the immune system. Additionally, there is a potential development of antimicrobial resistance. Catheters impregnated with antimicrobials, such as metal ions and antibiotics, have become popular and are now commercialized due to promising in vitro work but, in clinical trials these catheters have shown, at best, mixed results. Importantly, there is concern that this approach may not be a long-term solution given that the presence of antimicrobial compounds may drive development of resistance, especially when considering the host factors that coat urinary catheters could potentially inhibit or decrease pathogen interaction with antimicrobials.

Microbial adhesion to medical devices is common for hospital-acquired infections, including for urinary catheters. If not properly treated these infections cause complications and exacerbate antimicrobial resistance. Catheter use elicits bladder inflammation, releasing host serum-proteins, including fibrinogen (Fg), into the bladder, which deposit on the urinary catheter. E. faecalis uses fibrinogen as a scaffold to bind and persist in the bladder despite antibiotic treatments. Inhibition of fibrinogen-pathogen interaction significantly reduces infection.

Host clotting factor 1, fibrinogen (Fg), (a host glycoprotein) is important for surface adhesion and subsequent establishment of biofilms and persistence of CAUTIs in E. faecalis and Staphylococcus aureus infections. Targeting catheter protein deposition may reduce pathogen (e.g., uropathogen) colonization, creating an effective intervention. Fg is continuously released into the bladder lumen in response to mechanical damage to the urothelial lining caused by catheterization. Once in the lumen, Fg is deposited on the catheter, providing a platform for incoming uropathogens to attach and form biofilms. Biofilms are the most common underlying cause of bacteriuria and play an important role in promoting bladder colonization, microbial persistence, and systemic dissemination.

### I. Methods of Infusion of a Substrate with an Impregnation Fluid

Technologies described herein relate to methods and systems for infusing a substrate with an impregnation fluid. In some embodiments, a substrate can be impregnated partially (e.g., less than 100%) with an impregnation fluid. In some embodiments, a substrate can be impregnated 99% or less with an impregnation fluid (e.g., 95% or less, 90% or less, 85% or less, 80% or less, 75% or less, 70% or less, 65% or less, 50% or less). In certain embodiments, a substrate can be impregnated more than 1% with an impregnation fluid (e.g., 50% or more, 55% or more, 60% or more, 65% or more, 70% or more, 75% or more. 80% or more, 85% or more, 90% or more, 99.9% or more). In some embodiments, a substrate can be impregnated with from 1% to 99.9% with an impregnation fluid (e.g., from 50% to 99.9%, from 55% to 99.9%, from 60% to 99.9%, from 65% to 99.9%, from 70% to 99.9%, from 75% to 99.9%, from 85% to 99.9%, from 90% to 99.9%, from 1% to 90%, from 50% to 90%, from 55% to 90%, from 60% to 90%, from 65% to 90%, from 70% to 90%, from 85% to 90%, from 1% to 85%, from 50% to 85%, from 55% to 85%, from 60% to 85%, from 65% to 85%, from 70% to 85%, from 75% to 85%, from 80% to 85%, from 1% to 80%, from 50% to 80%, from 55% to 80%, from 60% to 80%, from 65% to 80%, from 70% to 80%, from 75% to 80%, from 1% to 75%, from 50% to 75%, from 55% to 75%, from 60% to 75%, from 65% to 75%, from 70% to 75%, from 1% to 70%, from 50% to 70%, from 55% to 70%, from 60% to 70%, from 65% to 70%, from 1% to 65%, from 50% to 65%, from 55% to 65%, from 60% to 65%, from 1% to 60%, from 50% to 60%, from 55% to 60%, from 1% to 55%, from 50% to 55%, from 1% to 50%).

In certain embodiments, a substrate can be infused from 90% to 99% with an impregnation fluid. In some embodiments, a maximum adhesion reduction (e.g., of proteins) with a minimal or substantially no impregnation fluid (e.g., silicone oil) overlayer occurs from to 95% impregnation.

In some embodiments, the degree of impregnation (e.g., percent impregnation, e.g., % Qₘₐₓ) for a given substrate is determined based on how much of an impregnation fluid is infused into a substrate. A change in mass of a substrate after infusion with an impregnation fluid can be used to determine the degree of impregnation. In some embodiments, the maximum amount of material that can be infused (e.g., 100% impregnated or substantially completely impregnated) into a substrate can be determined by monitoring the change in mass or weight of the substrate over time during the infusion process. In certain embodiments, for example, full impregnation is determined by measuring changes in mass or weight of a substrate and determining that no further, substantial changes in weight are observed. In certain embodiments, a fully impregnated substrate changes less than 10% (e.g., less than 5%, less than 1%) between weight measurements.

In certain embodiments, Qₘₐₓ is a maximum absorption capacity of a substrate. That is, Qₘₐₓ is an amount of impregnation fluid that is substantially the maximum amount of impregnation fluid that can be infused for a given infusion method, infusion conditions, or period of time. Qₘₐₓ is determined using the following equation:

Qₘₐₓ=(M₁−M₀)/M₀

where M₁ is a mass of the given material after having been fully impregnated and M₀ is the initial mass of the material. In certain embodiments, the absorption capacity of a substrate can be expressed as a percentage of Qₘₐₓ.

In certain embodiments, Qₘₐₓ can be calculated as the amount of impregnation fluid absorbed by a substrate such that no significant changes in mass of an impregnated substrate are observed over a given period of time. For example, Qₘₐₓ can be determined after relatively little to no change is observed in the mass of the substrate undergoing or having undergone infusion.

Infusing a substrate with an impregnation fluid can involve immersing or submerging a substrate in an impregnation fluid for a time period. In certain embodiments, a substrate is a polymeric (e.g., a cross-linked) substrate that is immersed in an impregnation fluid (e.g., silicone oil). In certain embodiments, a time period is at least 1 minute (e.g., at least 5 minutes, at least, 10 minutes, at least 30 minutes, at least 1 hour, at least 15 hours, at least 20 hours, at least 24 hours, at least 2 days, at least 5 days or more). In certain embodiments, a time period is dependent on the degree of impregnation desired. In certain embodiments, a time period is determined based on one or more physical properties of a substrate. For example, without limitation, substrate thickness, substrate density (e.g., crosslinking density), temperature (e.g., of an impregnation fluid), and substrate composition may effect infusion time and a rate at which a substrate is infused with an impregnation fluid.

In certain embodiments, infusing a substrate with an impregnation fluid results in an overlayer or immobilized layer of impregnation fluid on a surface of the substrate. In some embodiments, a substrate can be impregnated (e.g., fully or partially impregnated) with an impregnation fluid such that an overlayer of fluid or immobilized layer of fluid is created on a surface of the substrate due to infusion. As discussed herein, in certain embodiments. the presence of an overlayer or immobilized fluid layer is undesirable as it prevents the adhesion of substantially any proteins to surfaces of an impregnated substrate. Additionally, an overlayer of impregnation fluid may leach into a subject into which a substrate is implanted, resulting in negative effects on the health or a condition of the subject.

In certain embodiments, an overlayer or an immobilized or overlayer of impregnation fluid is removed from a surface of a substrate after infusion of the substrate with the impregnation fluid. In certain embodiments, an immobilized fluid or overlayer is removed mechanically and/or chemically from a substrate. In certain embodiments, mechanical removal of an overlayer or immobilized fluid layer involves contacting a surface of a substrate with a suitable device or implement to physically wipe the surface of the device. For example, in some embodiments, a surface of a substrate is wiped with an absorbent material (e.g., a Kimwipe™) to remove excess or immobilized fluid from a surface of an impregnated substrate. In some embodiments, mechanical removal results in removal of substantially all of the immobilized or excess impregnation fluid from a surface of a substrate. In certain embodiments, chemical removal of an overlayer or immobilized fluid layer results in dissolution of an overlayer or immobilized fluid layer (e.g., substantially complete dissolution of an overlayer or immobilized fluid layer). In certain embodiments, a solvent is used to chemically remove an overlayer or immobilized fluid layer. In certain embodiments, a solvent is any suitable solvent for dissolution of an overlayer or immobilized fluid layer. Based on the present disclosure, a person of skill in the art would understand solvents for dissolution of an overlayer or immobilized fluid layer.

### II. Impregnation Fluids

An impregnation fluid is any suitable fluid which can be infused (e.g., by diffusion) into a substrate in order to create a desired effect on absorption or adhesion to a surface of a substrate. In certain embodiments, an impregnation fluid is biocompatible (e.g., medical grade).

In certain embodiments, an impregnation fluid is or comprises a silicone oil (e.g., when the substrate is a polymeric substrate, e.g., PDMS). In certain embodiments, the viscosity of a silicone oil is greater than 0.65 cSt (e.g., greater than 10 cSt, greater than 50 cSt, greater than 1000 cSt). In certain embodiments, the viscosity of a silicone oil is less than 10,000 cSt (e.g., less than 1000 cSt, less than 50 cSt, less than 10 cSt). In certain embodiments, the viscosity of a silicone oil is from 0.65 cSt to 10,000 cSt (e.g., from 0.65 cSt to 1000 cSt, from 0.65 cSt to 50 cSt, from 0.65 cSt to 10 cSt, from 10 cSt to 1000 cSt, from 10 cSt to 50 cSt, from 50 cSt to 1000 cSt, from to 10,000 cSt, from 1000 cSt to 10,000 cSt). In certain embodiments, the viscosity of a silicone oil is about 50 cSt. In certain embodiments, the viscosity is a kinematic viscosity of the fluid. In certain embodiments, the viscosity of a silicone oil is measured using a viscometer (e.g., a u-tube viscometer, a falling-sphere viscometer, a vibrational viscometer, a rotational viscometer, an electromagnetically spinning-sphere viscometer). In certain embodiments, the viscosity is measured at about 25° C. using a viscometer.

In certain embodiments, an impregnation fluid comprises low-volatility polydimethylsiloxanes, cyclic polydimethylsiloxanes (e,g, cyclomethicones), silicone emulsions, silicone fluid blends, thermal silicone fluids, organic compatible silicone fluids (e.g., alkyl silicones (e.g., alkyl-methylsiloxane fluids), aryl-alkyl silicones), fluorosilicones, hydrophilic silicones (e.g. polyalkylene oxide silicones), polar silicones, ampliphilic silicones, low-temperature fluids (e.g. polydiethysiloxanes, silahydrocarbons, branched fluids, disiloxanes, trisiloxane fluids), naturally derived silicones (e.g., MonoAnisyl-terminated polydimethylsiloxane, limonenyl trisiloxane). In certain embodiments, a silicone oil comprises trimethoxy terminated polydimethylsiloxane. In certain embodiments, a silicone oil comprises repeating siloxane units and end-blocking siloxane units.

In certain embodiments, a silicone oil is a medical grade silicone oil. In some embodiments, the silicone oil comprises a silicone oil that is or is an equivalent of a Liveo™360 Medical Fluid. For example, without limitation, a silicone oil has a viscosity of about 20 cSt, 100 cSt, about 350 cSt, about 1000 cSt, or about 23,500 cSt. In certain embodiments, a silicone oil comprises a plurality of Liveo™360 Medical Fluids such that a fluid with an intermediate viscosity (e.g., a viscosity between one or more available viscosities) is achieved.

In certain embodiments, an impregnation fluid comprises one or more of the below listed trimethylsiloxy terminated polydimethysiloxanes or substantial equivalents thereof having the properties listed in Table 1 below:

| | | Viscosity | | | |
|---|---|---|---|---|---|
| Product | | Temp. | Pourpoint | Specific | Refractive |
| Code* | Viscosity | Coefficient | ° C. | Gravity | Index |
| DMS-T0 | .65 | .32 | −68 | .761 | 1.3750 |
| DMS-T01 | 1.0 | .37 | −85 | .818 | 1.3825 |
| DMS-T01.5 | 1.5 | .46 | −75 | .853 | 1.3880 |
| DMS-T02 | 2.0 | .48 | −80 | .873 | 1.3900 |
| DMS-T03 | 3.0 | .51 | −70 | .898 | 1.3935 |
| DMS-T05 | 5.0 | .54 | −65 | .918 | 1.3970 |
| DMS-T07 | 7.0 | .55 | −65 | .930 | 1.3980 |
| DMS-T11 | 10 | .56 | −65 | .935 | 1.3990 |
| DMS-T12 | 20 | .59 | −65 | .950 | 1.4000 |
| DMS-T15 | 50 | .59 | −65 | .960 | 1.4015 |
| DMS-T21 | 100 | .60 | −65 | .966 | 1.4025 |
| DMS-T22 | 200 | .60 | −60 | .968 | 1.4030 |
| DMS-T23 | 350 | .60 | −60 | .970 | 1.4031 |
| DMS-T25 | 500 | .60 | −55 | .971 | 1.4033 |
| DMS-T31 | 1,000 | .61 | −50 | .971 | 1.4034 |
| DMS-T35 | 5,000 | .61 | −48 | .973 | 1.4035 |
| DMS-T41 | 10,000 | .61 | −48 | .974 | 1.4035 |
| DMS-T41.2 | 12,500 | .61 | −46 | .974 | 1.4035 |
| DMS-T43 | 30,000 | .61 | −43 | .976 | 1.4035 |
| DMS-T46 | 60,000 | .61 | −42 | .976 | 1.4035 |
| DMS-T51 | 100,000 | .61 | −41 | .977 | 1.4035 |
| DMS-T53 | 300,000 | .61 | −41 | .977 | 1.4035 |
| DMS-T56 | 600,000 | .61 | −41 | .978 | 1.4035 |
| DMS-T61 | 1,000,000 | .62 | −39 | .978 | 1.4035 |
| DMS-T63 | 2,500,000 | .62 | −38 | .978 | 1.4035 |
| DMS-T72 | 20,000,000 | .62 | −35 | .979 | 1.4035 |

is

| | Coeff. of | Thermal | | | | | |
|---|---|---|---|---|---|---|---|
| | Thermal | Conductivity Cal/ | Surface | Dielectric | Dielectric | Flashpoint | Molecular |
| Product Code* | Expansion ×10⁻⁴ | cm · sec. ×10⁻⁴° C. | Tension | Constant | Strength | C. ° | Weight |
| DMS-T00 | 13.4 | 2.4 | 15.9 | 2.20 | 300 | −1 | 162 |
| DMS-T01 | 13.4 | 2.4 | 17.4 | 2.30 | 350 | 39 | 237 |
| DMS-T01.5 | 13.4 | 2.5 | 18.0 | 2.39 | 350 | 63 | 340 |
| DMS-T02 | 11.7 | 2.6 | 18.7 | 2.45 | 350 | 79 | 410 |
| DMS-T03 | 11.4 | 2.7 | 19.2 | 2.50 | 350 | 100 | 550 |
| DMS-T05 | 11.2 | 2.8 | 19.7 | 2.60 | 375 | 135 | 770 |
| DMS-T07 | 11.0 | 3.0 | 19.9 | 2.65 | 375 | 150 | 950 |
| DMS-T11 | 10.8 | 3.2 | 20.1 | 2.68 | 375 | 163 | 1,250 |
| DMS-T12 | 10.7 | 3.4 | 20.6 | 2.72 | 375 | 232 | 2,000 |
| DMS-T15 | 10.6 | 3.6 | 20.8 | 2.75 | 400 | 285 | 3,780 |
| DMS-T21 | 9.3 | 3.7 | 20.9 | 2.75 | 400 | 315 | 5,970 |
| DMS-T22 | 9.3 | 3.7 | 21.0 | 2.75 | 400 | 315 | 9,430 |
| DMS-T23 | 9.3 | 3.8 | 21.1 | 2.75 | 400 | 315 | 13,650 |
| DMS-T25 | 9.3 | 3.8 | 21.1 | 2.75 | 400 | 315 | 17,250 |
| DMS-T31 | 9.3 | 3.8 | 21.2 | 2.75 | 400 | 315 | 28,000 |
| DMS-T35 | 9.3 | 3.8 | 21.3 | 2.75 | 400 | 315 | 49,350 |
| DMS-T41 | 9.3 | 3.8 | 21.5 | 2.75 | 400 | 315 | 62,700 |
| DMS-T41.2 | 9.3 | 3.8 | 21.5 | 2.75 | 400 | 315 | 67,700 |
| DMS-T43 | 9.3 | 3.8 | 21.5 | 2.75 | 400 | 315 | 91,700 |
| DMS-T46 | 9.2 | 3.8 | 21.5 | 2.75 | 400 | 315 | 116,500 |
| DMS-T51 | 9.2 | 3.8 | 21.5 | 2.75 | 400 | 321 | 139,000 |
| DMS-T53 | 9.2 | 3.8 | 21.5 | 2.75 | 400 | 321 | 204,000 |
| DMS-T56 | 9.2 | 3.8 | 21.6 | 2.75 | 400 | 321 | 260,000 |
| DMS-T61 | 9.2 | 3.8 | 21.6 | 2.75 | 400 | 321 | 308,000 |
| DMS-T63 | 9.2 | 3.8 | 21.6 | 2.75 | 400 | 321 | 423,000 |
| DMS-T72 | 9.2 | 3.8 | 21.6 | 2.75 | 400 | 321 | >500,000 |

A person of skill in the art would, in light of the teachings in the present disclosure, understand the provided measurements and be able to extrapolate equivalent silicone fluids based on the disclosure.

As would be understood by a person of skill in the art, the value, viscosity-temperature coefficient (VTC), is a measure of the change of fluid viscosity over the temperature range 38° C. to 99° C. The VTC can be measured using the following equation: VTC=1−(viscosity at 99° C./viscosity at 38° C.).

Thus, the lower the V.T.C. the less the change in viscosity over the temperature range.

In certain embodiments, an impregnation fluid is or comprises a silicone fluid. A table of silicone fluid classes and associated physical and chemical properties are presented below in Table 2:

TABLE 2
List of exemplary silicone fluid classes.

| | | | | | Organic |
|---|---|---|---|---|---|
| | | | | Thermal | Compatible |
| | | Comment | Conventional | Silicone | Silicone |
| Property | | on Property | Silicone Fluids | Fluids | Fluids |
| Thermal | High Temp ° C. | 1000 hours | 175° C. | 260° C. | 150° C. |
| Properties | | in air, max. | | | |
| | High Temp ° C. | indefinite O₂ | 200° C. | 280° C. | — |
| | | free, max. | | | |
| | Low Temp ° C. | pour point, | −70° C. | −73° | −50° |
| | | low value | | | |
| Rheological | Viscosity, cSt. | range | 3-2.5 × 10⁶ | 50-3.0 × 10⁵ | 500-1 × 10⁴ |
| Properties | Visc.-temp. coeff. | low value | 0.51 | 0.61 | 0.75 |
| Electrical | Dielectric | range | 360-400 | 400-420 | — |
| Properties | Strength | | | | |
| | volts/mil | | | | |
| | Dielectric | range, | 2.50-2.77 | 2.78-2.95 | 2.5-3.0 |
| | Constant | 100 Hz | | | |
| Mechanical | Compressibility, | @ 20,000 | 9.1 | 5.5 | approx. 5-8 |
| Properties | % | psi | | | |
| | Density, g/cc | | 0.90-0.98 | 0.98-1.15 | 0.88-1.04 |
| Compatibility | Water solubility | | insoluble | insoluble | insoluble- |
| Properties | | | | | partial |
| | Hydrocarbon | aromatic/ | soluble/partial | soluble/ | soluble/ |
| | solubility | aliphatic | | soluble | soluble |
| Optical | Refractive Index n_{d}²⁵ | range | 1.393-1.403 | 1.428-1.582 | 1.443-1.493 |
| Properties | | | | | |
| Release & | Surface Tension, | range | 19.2-21.6 | 20.5-28.5 | 22.0-39.5 |
| Wettability | dynes/cm | | | | |
| Properties | | | | | |
| Wear/Lubricity | Four ball wear, | | 2-3 | 1.8-2.5 | 0.7 |
| Properties | mm at 75° C., 40 | | | | |
| | kg. load steel on | | | | |
| | steel, one hr. | | | | |

| | | Hydrophilic | |
|---|---|---|---|
| | | and | Low |
| | Fluorosilicone | Polar Silicone | Temperature |
| Property | Fluids | Fluids | Silicone Fluids |
| Thermal | 190° C. | 135° C. | 235° C. |
| Properties | 230° C. | — | 260° C. |
| | −47° C. | −50° C. | −100° C. |
| Rheological | 80-1 × 10⁴ | 20-5,000 | 4-400 |
| Properties | 0.84 | — | 0.5 |
| Electrical | 175-200 | — | 300-400 |
| Properties | 6.95-7.35 | — | — |
| Mechanical | 7.5 | approx. 7 | 11.9 |
| Properties | 1.25-1.30 | 1.00-1.07 | 0.76-1.09 |
| Compatibility | insoluble | insoluble- | insoluble |
| Properties | | soluble | |
| | insoluble/ | partial/ | soluble/soluble |
| | insoluble | insoluble | |
| Optical | 1.336-1.387 | 1.441-1.454 | 1.335-1.588 |
| Properties | | | |
| Release & | 25.7-28.7 | 23.6-27.0 | 15.9-26.7 |
| Wettability | | | |
| Properties | | | |
| Wear/Lubricity | 0.8 | 2-6 | 0.9-2.5 |
| Properties | | | |

In certain embodiments, an impregnation fluid is a hydrophilic liquid or a hydrophilic ionic liquid (e.g., when the substrate is a hydrogel). In some embodiments, an impregnation fluid is a liquid comprising ammonia, alcohol(s), one or more amides (e.g., urea), or carboxylic acid (e.g., acetic acid). In certain embodiments, a hydrophilic liquid or a hydrophilic ionic liquid is used when altering adhesion and/or adsorption of hydrophilic proteins.

### III. Substrates

A substrate described herein can be or comprise any suitable substrate that can be infused with an impregnation fluid to achieve desired adhesion or other surface properties. In some embodiments, substrates described herein are polymeric substrates. In some embodiments, a polymeric substrate is cross-linked (e.g., cross-linked polydimethylsiloxane (PDMS)). In some embodiments, a substrate is biocompatible (e.g., does not harm biological tissue).

In certain embodiments, a polymeric substrate is or comprises silicone. In certain embodiments a silicone is polydimethylsiloxane (PDMS).

In certain embodiments, a substrate is or comprises a hydrogel, poly(acrylic acid), poly(vinyl alcohol), poly(vinylpyrrolidone), poly(ethylene glycol), polyacrylamide, or a polysaccharide hydrogels or a modified version or equivalent thereof. In certain embodiments, a substrate is or comprises an organogel. In certain embodiments, an organogel is comprised of anthracene, anthracene, anthraquinone or steroid-based molecules.

In some embodiments, a substrate is or comprises a hydrogel. In some embodiments, a substrate being or comprising a hydrogel is used to coat a part of a device (e.g., a medical device) where there is a high likelihood of unwanted adhesion of proteins. For example, in some embodiments, joints or parts of a device create turbulent flow, which creates a higher likelihood of proteins adhering to the device at or near these locations. Coating the device with a hydrogel as described herein may help to control protein adhesion or adsorption to the device.

In certain embodiments, the degree of cross-linking of a polymer substrate can be used to control impregnation of the fluid. For example, in certain embodiments, a lower cross-linking density is associated with a higher amount of fluid being allowed to impregnate the substrate.

By way of non-limiting example, certain materials useful in creating a polymeric substrate include, but are not limited to, natural and synthetic elastomers such as Ethylene Propylene Diene Monomer (EPDM, a terpolymer of ethylene, propylene and a diene component)), natural and synthetic polyisoprenes such as cis-1,4-polyisoprene natural rubber (NR) and trans-1,4-polyisoprene gutta-percha, isoprene rubber, chloroprene rubber (CR), such as polychloroprene, Neoprene, and Baypren, Butyl rubber (copolymer of isobutylene and isoprene), Styrene-butadiene Rubber (copolymer of styrene and butadiene, SBR), Nitrile rubber (copolymer of butadiene and acrylonitrile, NBR), also called Buna N rubbers, Epichlorohydrin rubber (ECO), Polyacrylic rubber (ACM, ABR), Fluoroelastomers (FKM, and FEPM) Viton, Tecnoflon, Fluorel, Aflas and Dai-El, Perfluoroelastomers (FFKM) Tecnoflon PFR, Kalrez, Chemraz, Perlast, Polyether block amides (PEBA), Chlorosulfonated polyethylene (CSM), (Hypalon), Ethylene-vinyl acetate (EVA), Polybutadiene, Polyether Urethane, Perfluorocarbon Rubber, Fluoronated Hydrocarbon (Viton), silicone, fluorosilicone, polyurethane, polydimethylsiloxane, vinyl methyl silicone, and their composite materials where one or more of such exemplary polymers are compounded with other filler materials such as carbon black, titanium oxide, silica, alumina, nanoparticles, and the like.

### IV. Infusion of Substrates with Impregnation Fluids

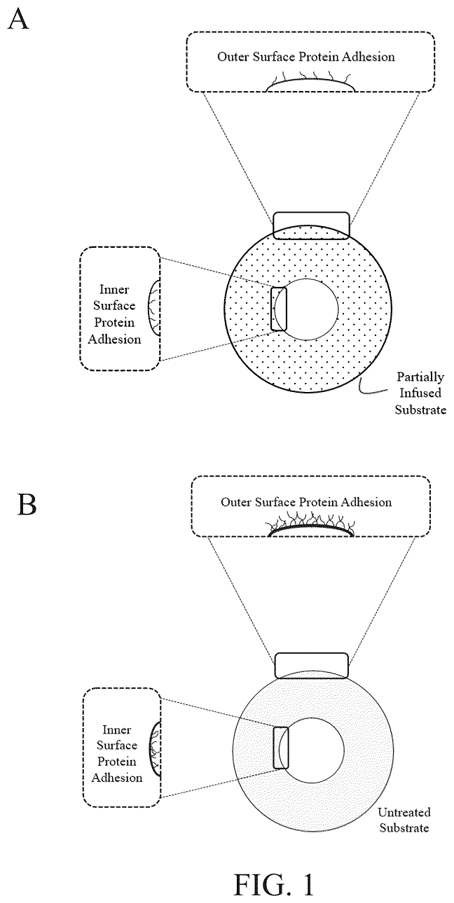

As described herein, substrates are infused with an impregnation fluid in order to alter adhesion to the substrate. In certain embodiments, an impregnation fluid reduces adhesion and/or adsorption of proteins to the surface of a substrate. FIG. 1, panel A is an illustrative embodiment of a partially silicone-oil impregnated indwelling urinary catheter. FIG. 1, panel B is an illustrative embodiment of an untreated urinary catheter. As shown in FIG. 1, panel A, partial impregnation of a urinary catheter with a silicone oil results in a decrease in adhesion of protein to the outer and inner surfaces of the catheter depicted. In the absence of infusion, there is an overall increase in protein adhesion to the inner and outer surfaces of the substrate.

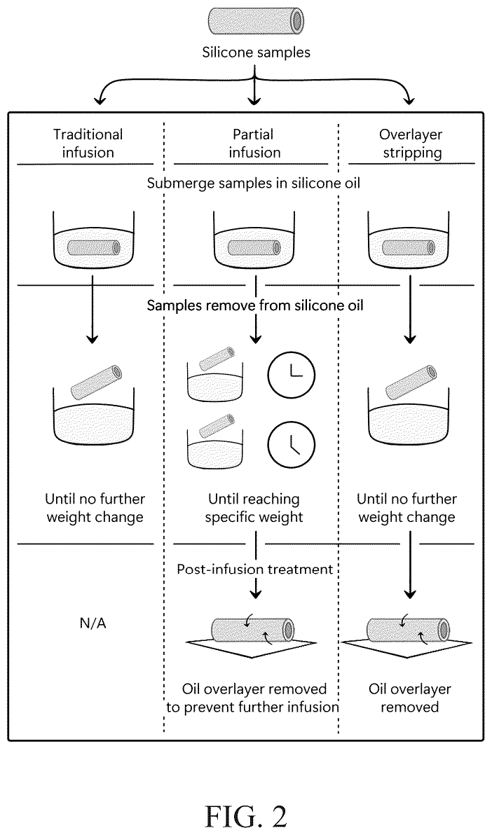

In order to achieve certain desirable effects, in some embodiments, the surface of a substrate does not have an overlayer or immobilized layer of impregnation fluid. FIG. 2 is a series of schematics illustrating different methods of infusing a substrate (e.g., a silicone substrate) with an impregnation fluid, according to an illustrative embodiment. The first method shows “traditional infusion”, which results in creation of an overlayer or immobilized layer of impregnation fluid on a surface of a substrate. For example, in an embodiment of traditional infusion, a substrate (e.g., a silicone substrate) is submerged or immersed in an impregnation fluid (e.g., silicone oil) for a period of time until no further weight change is observed. As a result, a free overlayer of impregnation fluid is present on the surface of the substrate. In “partial infusion”, a substrate (e.g., a silicone substrate) is submerged or immersed in an impregnation fluid (e.g., silicone oil) for a period of time until a specific weight of the partially impregnated substrate is reached. A change in mass of the substrate after infusion with an impregnation fluid can be used to determine the degree of impregnation. In some embodiments, the maximum amount of material that can be infused (e.g., 100% impregnated or substantially completely impregnated) into a substrate can be determined by monitoring the change in mass of the material over time during the infusion process. In some embodiments, partial infusion is also described as a percentage absorption capacity of a substrate (e.g., % Qₘₐₓ). In some embodiments, Qₘₐₓ is the absorption capacity of the substrate. In some embodiments, 100% Qₘₐₓ is indicative that a substrate has achieved its maximum absorption of an impregnation fluid. After infusion, a free overlayer of fluid is removed from a surface of a substrate. In some embodiments, a free overlayer of fluid is removed mechanically and/or chemically (e.g., as described herein).

Another method for removal of a free overlayer of impregnation fluid is known as “overlayer stripping” (e.g., as shown in FIG. 2). In overlayer stripping, a substrate is submerged or immersed in an impregnation fluid for a period of time to substantially fully impregnate the substrate with the impregnation fluid. In some embodiments, full impregnation is determined by measuring changes in mass or weight of a substrate and determining that no further, substantial changes in weight are observed. In some embodiments, a fully impregnated substrate changes less than 10% (e.g., less than 5%, less than 1%, or less) between weight measurements. After infusion, a free overlayer of fluid is removed mechanically and/or chemically (e.g., as described herein).

In certain embodiments, adhesion and/or adsorption of proteins to the substrate can be increased or reduced depending on an impregnation fluid used or degree of impregnation. In certain embodiments, it is desirable to increase adhesion and/or adsorption of uncharged proteins to the substrate. In certain embodiments, adhesion and/or adsorption of uncharged proteins not involved in an immune response of the subject in which the substrate is increased. Examples of proteins having a more neutral surface charge include, but are not limited to, UDP-glucose 6-dehydrogenase (UGDH), filamin B (FLNB), and Proteasome 20S Subunit Beta 5 (PSMB5)). In certain embodiments, it is desirable to reduce adhesion for proteins involved in an immune response and/or responsible for adhesion of bacteria to the substrate. Examples of proteins that are responsible for adhesion of bacteria to the substrate include, but are not limited to, fibrinogen and serum albumin.

Without wishing to be bound to any particular theory, infusion of silicone oil into a polymeric substrate (e.g., PDMS) may result in free silicone oil chains diffusing through the polymeric substrate to places within the bulk (e.g., the polymer bulk) and on the surface of the polymeric substrate which possess a localized charge (e.g., a positive or negative charge). Once at the charge location, the silicone oil neutralizes the localized charges, resulting in altered adhesion properties as compared to untreated substrates.

In certain embodiments, adhesion or absorption of proteins to a surface of a silicone (e.g., PDMS) substrate is reduced when impregnated with a sufficient amount of silicone oil. In certain embodiments, the adhesion or adsorption of proteins involved in an immune response or responsible for adhesion of pathogens (e.g., bacteria) to a surface is reduced (e.g., as compared to an untreated substrate). Proteins for which adhesion is reduced upon impregnation of a silicone substrate with silicone oil includes fibrinogen and serum albumin. In certain embodiments, adhesion or absorption of proteins to a surface of a silicone substrate is increased when impregnated with a sufficient amount of silicone oil. For example, proteins that are not involved in an immune response of a subject can be increased when the substrate is infused with silicone oil. Exemplary proteins for which adhesion is increased upon impregnation of a PDMS substrate with silicone oil includes UDP-glucose 6-dehydrogenase (UGDH), filamin B (FLNB), and Proteasome 20S Subunit Beta 5 (PSMB5).

### IV. Medical Devices

In certain embodiments, substrates described herein are used with or form all or a part of a medical device. The substrates described herein can be used in any suitable medical device which would benefit from altered surface adhesion properties. In certain embodiments, the medical device is an indwelling medical device. Indwelling medical devices include, but are not limited to, urinary catheters, vascular access devices, endotracheal tubes, tracheostomies, feeding tubes (e.g., enteral feeding tubes), wound drains, and the like. In certain embodiments, medical devices include implantable medical devices (e.g., a device which is either wholly or partially inserted, e.g., surgically, into the body). In certain embodiments, medical devices described herein which comprise the substrate are biocompatible or have portions thereof which are biocompatible (e.g., portions including the medical device). In certain embodiments, medical devices used with methods and substrates described herein are pre-fabricated urinary catheters which are amenable to modification using the techniques described herein.

In certain embodiments, a medical device is or comprises a tube having an inner surface (i.e., interior) and an outer surface (i.e., exterior). In certain embodiments, a substrate is found on the inner surface of the tube (e.g., a portion in contact with a bodily fluid). In certain embodiments, a substrate is found on the outer surface of the tube (e.g., a portion in contact with a bodily fluid and/or tissue). In certain embodiments, a substrate forms both the inner and outer surfaces of the tube (e.g., as in a catheter). In certain embodiments, a substrate contacts biological tissue and/or biological fluids. Biological fluids include, but are not limited to, urine, blood, interstitial fluids, saliva, intraperitoneal fluids (e.g., abdominal fluids, ascites), gastric juices, and the like.

In certain embodiments, a medical device or portion thereof comprising the polymeric substrate has reduced adhesion for pathogens (e.g., bacteria) to the medical device. Reduced adhesion of pathogens to the substrate is a result of infusing the substrate with a suitable impregnation fluid as described herein. Pathogens include, but are not limited to, uropathogens such as E. faecalis, C. albicans, uropathogenic Escherichia coli, Pseudomonas aeruginosa, A. baumannii, and Klebsiella pneumoniae.

### VI. Experimental Example 1

In an embodiment discussed herein, host-protein deposition was reduced using liquid-infused (e.g., oil-infused, silicone-oil impregnated) catheters resulting in decreased colonization of bacteria on catheters, in bladders, and dissemination in vivo. Furthermore, proteomics revealed a significant decrease in deposition of host-secreted proteins on liquid-infused catheter surfaces. Findings presented herein suggest targeting microbial binding scaffolds may be an effective, antibiotic-sparing intervention for use against catheter-associated urinary tract infections and other medical device infections.

Reducing availability of binding scaffolds, in this case fibrinogen (Fg), decreases microbial colonization in a catheterized bladder. A mouse model of CAUTI using a diverse panel of uropathogens, including E. faecalis, C. albicans, uropathogenic Escherichia coli, Pseudomonas aeruginosa, A. baumannii, and Klebsiella pneumonia, found all uropathogens bound more extensively to catheters with Fg present.

Anti-fouling modifications of the catheter were used to inhibit the deposition of fibrinogen (Fg). In the current embodiment, the anti-fouling modification is liquid infused silicone (LIS) (i.e., silicone infused with a silicone oil). LIS is simpler to make, more stable and more cost effective than other anti-fouling polymer modifications. Additionally, LIS reduces clotting in central lines and infection in skin implants. As disclosed herein, LIS-catheters reduced Fg deposition and microbial binding not only in vitro but also in vivo. Furthermore, LIS-catheters significantly decrease host-protein deposition when compared to unmodified (UM)-catheters as well as reducing catheter-induced inflammation. Without wishing to be bound to any particular theory, the findings presented herein suggest that targeting host-protein deposition on catheter surfaces and the use of LIS-catheters are plausible strategies for reducing instances of CAUTI.

Uropathogens Interact with Fg During CA UTI.

Due to the interaction between Fg and some uropathogens as well as Fg accumulation on catheters over time in humans and mice, potential interactions of E. faecalis OG1RF (positive control) uropathogenic E. coli UTI89, P. aeruginosa PAO1, K. pneumoniae TOP52, A. baumannii UPAB1, and C. albicans SC5314 with Fg were assessed in vivo, using a CAUTI mouse model. Mice catheterized and infected with the respective uropathogen were sacrificed at 24 hours post infection (hpi). Catheters and bladders were harvested, stained, and imaged. Visual and quantitative analysis of the catheters showed uropathogens co-localizing strongly with Fg deposits, which demonstrates a preference of uropathogens for Fg.

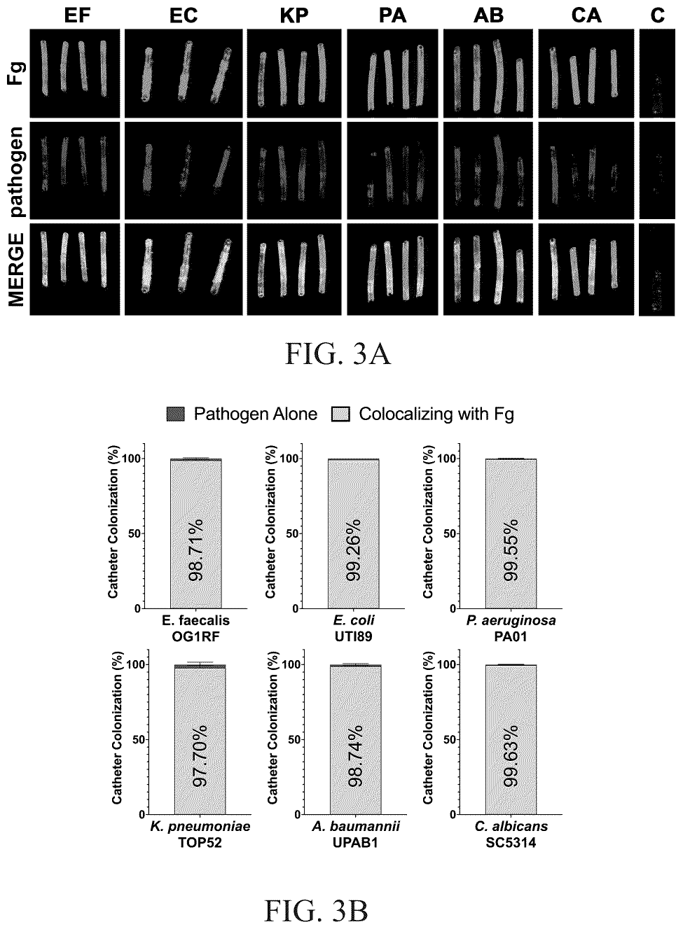

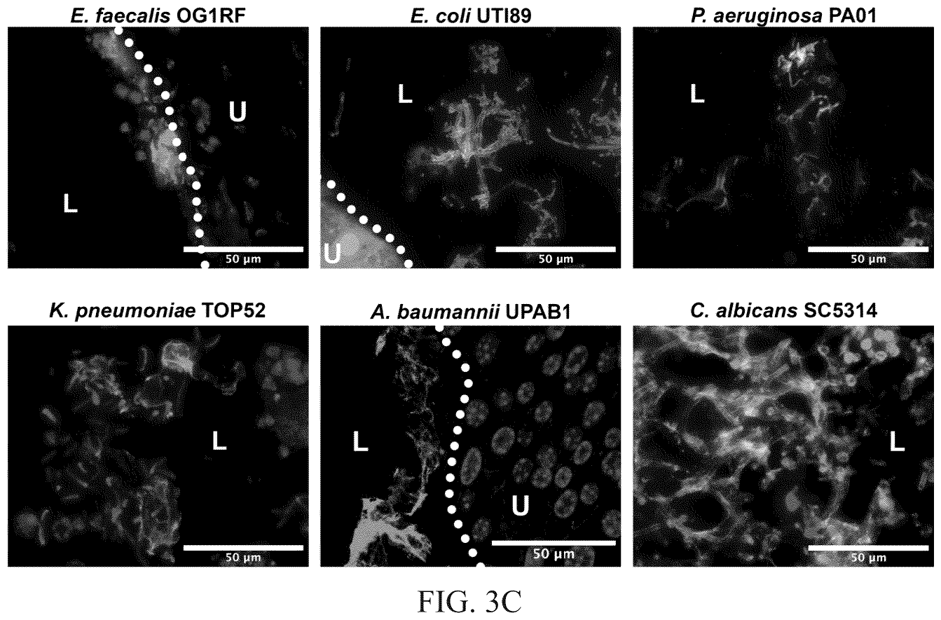

FIG. 3A is a series of images of urinary catheters extracted from mice, which were stained with immunofluorescent markers. The top row of images shows catheters stained for fibrinogen (Fg) deposition. Green coloration is representative of fibrinogen deposition on the surface of the catheter. The center row shows catheters stained with a red coloration to identify pathogens bound to the catheter. The respective pathogen is labeled along the horizontal axis as follows: EF (E. faecalis OG1RF), EC (E. coli UTI89), PA (P. aeruginosa PA01), AB (A. baumannii, UPAB1), CA (C. albicans SC5314). The C label designates a control catheter, which was not implanted into the mouse. FIG. 3B is representative of the degree of overlap between the pathogen image and fibrinogen image. The red portion of the bar is indicative of the portion of the stained pathogen that does not overlap with the fibrinogen (Fg). The yellow portion of the bar is indicative of the portion of the stained pathogen that does co-localize with fibrinogen (Fg). A completely red bar would indicate that the pathogen does not co-localize with fibrinogen, while a completely yellow bar would indicate complete colocalization of fibrinogen and pathogen. The portion of the pathogen colocalizing with fibrinogen is listed as follows: E. faecalis OG1RF (positive control)—98.71%, uropathogenic E. coli UTI89-99.26%, P. aeruginosa PAO1—99.55%, K. pneumoniae TOP52—97.70%, A. baumannii UPAB1—98.74%, and C. albicans SC5314—99.63%. For all graphs, error bars show the standard error of the mean (SEM). Between 3 and 5 replicates of n=4-12 each were performed for each pathogen and condition.

FIG. 3C is a series of immunofluorescence (IF) images of mouse bladder tissue sections. IF analysis of bladder sections showed that all uropathogens interact with Fg on the bladder urothelium or in the lumen during CAUTI. Each panel of FIG. 3C shows a representative image from a single bladder illustrating the interaction of uropathogens (red), Fg (green), and nuclei (blue) on the bladder urothelium (U) and in the lumen (L).

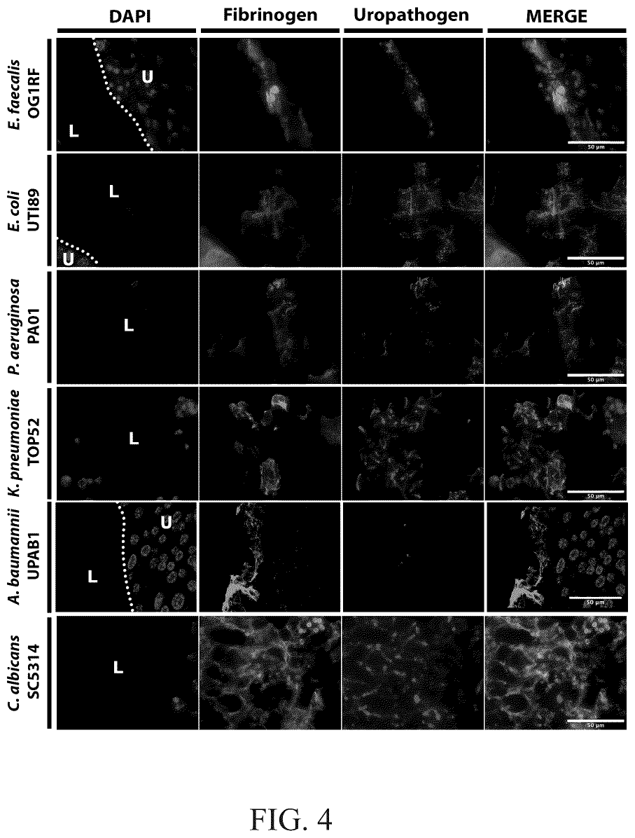

FIG. 4 is a series of color separated images corresponding to the individual panels of FIG. 3C. Mice were implanted and infected with 1×10⁶ CFU of the respective uropathogens. At 24 hpi, bladders tissues were harvested, fixed, and parafilm-embedded. Bladder tissue sections were subjected to IF analysis. Antibody staining was used to detect Fg (anti-Fg; green), uropathogens (red), and cell nuclei (DAPI; blue). Scale bars are 50 μm. Magnification of the images is 100×.

### Fg on Urinary Catheter Material Enhances Microbial Binding.

Based on in vivo findings, it was assessed whether Fg could promote initial binding of the uropathogens to silicone catheters. In addition to Fg, bovine serum albumin (BSA) was tested since serum albumin is one of the most abundant protein on human and mouse urinary catheters as shown in Table 3. Table 3 is a list of proteins found on LI and UM mouse catheters infected with E. faecalis OG1RF. The average number of peptides for each protein found on 10 mouse catheters sorted by greatest abundance on the UM catheter. Table 3 is also found in Andresen et al. Inhibiting host-protein deposition on urinary catheters reduces associated urinary tract infections. eLife 2022; 11:e75798. DOI: doi.org/10.7554/eLife.75798, which is incorporated by reference in its entirety.

TABLE 3
List of proteins found on LI and UM mouse catheters
infected with E. faecalis OG1RF.

| | | | | Decrease |
|---|---|---|---|---|
| | | UM | LI | in |
| | | Catheter | Catheter | Binding |
| Protein IDs | Protein names | Average StDev | Average StDev | (%) |
| P01027 | Complement C3 | 30.50 ± 3.70 | 10.80 ± 5.55 | 64.59 |
| H3BKW9 | | | | |
| H3BL60 | | | | |
| CON_Q2UVX4 | | | | |
| P07724 | Serum albumin | 30.38 ± 3.02 | 15.80 ± 2.82 | 47.98 |
| Q921I1 | Serotransferrin | 25.38 ± 4.34 | 9.10 ± 2.28 | 64.14 |
| D3YYR8 | | | | |
| F7BAE9 | | | | |
| E9Q2Q7 | | | | |
| F7CJN9 | | | | |
| E9Q939 | | | | |
| CON_Q29443 | | | | |
| CON_Q0IIK2 | | | | |
| CON_Q2HJF0 | | | | |
| E9PV24 | Fibrinogen alpha, | 22.00 ± 8.98 | 8.70 ± 4.31 | 60.45 |
| CON_P02672 | beta and gamma | | | |
| Q8K0E8 | | | | |
| Q3UER8 | | | | |
| Q8VCM7 | | | | |
| Q8VDD5 | Myosin-9 | 18.86 ± 15.02 | 6.70 ± 6.57 | 64.47 |
| Q5SV64 | | | | |
| Q3UH59 | | | | |
| Q61879 | | | | |
| A0A2R8VKI5 | | | | |
| Q8BXF2 | | | | |
| A0A140LI60 | | | | |
| E9Q264 | | | | |
| A2AQP0 | | | | |
| Q00623 | Apolipoprotein | 15.00 ± 2.07 | 6.50 ± 1.35 | 56.67 |
| A0A1L1STX7 | A-I | | | |
| CON_P15497 | | | | |
| Q61838 | Alpha-2- | 13.88 ± 4.76 | 3.33 ± 2.40 | 75.98 |
| D3YUI3 | macroglobulin | | | |
| Q6GQT1 | | | | |
| P20152 | Vimentin | 11.00 ± 9.77 | 3.25 ± 3.37 | 70.45 |
| A0A0A6YWC8 | | | | |
| A2AKJ2 | | | | |
| P31001 | | | | |
| D3YZ35 | | | | |
| A0A0R4J036 | | | | |
| P46660 | | | | |
| P08551 | | | | |
| P08553 | | | | |
| P11679 | Keratin, type II | 10.75 ± 1.49 | 9.10 ± 4.41 | 15.35 |
| CON_Q9H552 | cytoskeletal 8 | | | |
| Q91X72 | Hemopexin | 10.50 ± 1.93 | 4.30 ± 2.45 | 59.05 |
| A0A1B0GS57 | | | | |
| B7FAV1 | Filamin-A | 10.29 ± 9.78 | 1.00 ± 1.69 | 90.28 |
| B7FAU9 | | | | |
| Q8BTM8 | | | | |
| F6XC15 | | | | |
| Q8VHX6 | | | | |
| F6Z2C0 | | | | |
| J3JS91 | | | | |
| A8DUK4 | Hemoglobin | 10.00 ± 3.46 | 5.90 ± 2.28 | 41.00 |
| P02088 | subunit beta-1 | | | |
| E9Q223 | | | | |
| P02089 | | | | |
| CON_Q3SX09 | | | | |
| CON_P02070 | | | | |
| P02104 | | | | |
| P13020 | Gelsolin | 9.88 ± 1.55 | 4.90 ± 2.81 | 50.38 |
| A0A0J9YUQ8 | | | | |
| CON_Q3SX14 | | | | |
| A6PWS5 | | | | |
| A0A0J9YUJ8 | | | | |
| P20918 | Plasminogen | 9.38 ± 3.62 | 1.70 ± 1.83 | 81.87 |
| CON_P06868 | | | | |
| P63260 | Actin, | 9.25 ± 3.54 | 6.00 ± 1.63 | 35.14 |
| P60710 | cytoplasmic 2 | | | |
| E9Q5F4 | | | | |
| G3UZ07 | | | | |
| E9Q1F2 | | | | |
| G3UYG0 | | | | |
| E9Q606 | | | | |
| A0A1D5RM20 | | | | |
| B1ATY1 | | | | |
| Q8BFZ3 | | | | |
| F8WGM8 | | | | |
| E9Q2D1 | | | | |
| F6WX90 | | | | |
| V9GXQ2 | | | | |
| B7ZNJ1 | Fibronectin | 8.88 ± 5.25 | 1.33 ± 1.73 | 84.98 |
| A0A087WS56 | Anastellin | | | |
| B9EHT6 | | | | |
| A0A087WSN6 | | | | |
| Q3UHL6 | | | | |
| A0A087WR50 | | | | |
| P11276 | | | | |
| Q4KL80 | | | | |
| A0A087WSU6 | | | | |
| A0A087WS99 | | | | |
| P28665 | Murinoglobulin-1 | 8.50 ± 3.12 | 1.22 ± 0.97 | 85.62 |
| P28666 | | | | |
| A0A0N4SVU1 | | | | |
| CON_ENSEMB | | | | |
| L:ENSBTAP0000 | | | | |
| 0024146 | | | | |
| A2BIN1 | Major urinary | 8.50 ± 3.02 | 6.40 ± 1.35 | 24.71 |
| Q4FZE8 | protein 6 | | | |
| A2AKN9 | | | | |
| A2BIM8 | | | | |
| P02762 | | | | |
| P04938 | | | | |
| A9C497 | | | | |
| A9C496 | | | | |
| A2CEK7 | | 7.88 ± 3.56 | 5.70 ± 1.57 | 27.62 |
| P08071 | Lactotransferrin | 7.88 ± 7.77 | 2.70 ± 3.65 | 65.71 |
| A0A0G2JDN0 | | | | |
| A0A0G2JGN1 | | | | |
| A0A0G2JFM6 | | | | |
| A0A0G2JEA3 | | | | |
| P11589 | Major urinary | 7.88 ± 3.44 | 5.60 ± 1.51 | 28.89 |
| | protein 2 | | | |
| A2AE89 | Glutathione S- | 7.63 ± 1.60 | 7.00 ± 4.40 | 8.20 |
| P10649 | transferase Mu 1 | | | |
| F6WHQ7 | | | | |
| P19639 | | | | |
| D3YVP6 | | | | |
| E9QAC8 | | | | |
| D3YVP5 | | | | |
| Q80W21 | | | | |
| F6Y363 | | | | |
| D3YZ29 | | | | |
| D3YVP8 | | | | |
| G5E8M7 | | | | |
| O35660 | | | | |
| D3YVP9 | | | | |
| E9PV63 | | | | |
| E9PVM7 | | | | |
| P48774 | | | | |
| Q61233 | Plastin-2 | 7.50 ± 7.37 | 1.33 ± 1.87 | 82.22 |
| A0A1C7CYV0 | | | | |
| B1AX58 | | | | |
| Q99K51 | | | | |
| D3YZ25 | | | | |
| D3YVW8 | | | | |
| D3Z7D9 | | | | |
| D3Z311 | | | | |
| Q3V0K9 | | | | |
| G3X9T8 | Ceruloplasmin | 7.50 ± 2.45 | 0.50 ± 0.53 | 93.33 |
| G3X8Q5 | | | | |
| E9PZD8 | | | | |
| Q61147 | | | | |
| G3UXG1 | | | | |
| G3UWP5 | | | | |
| G3UZ53 | | | | |
| A2AKN8 | | 7.50 ± 3.51 | 5.50 ± 1.27 | 26.67 |
| E9PVW0 | | | | |
| B8JI96 | | | | |
| A2BIN0 | | | | |
| P11591 | | | | |
| P01132 | Pro-epidermal | 7.38 ± 7.58 | 2.89 ± 1.83 | 60.83 |
| A0A0G2JDT8 | growth factor | | | |
| A0A0G2JF92 | | | | |
| A0A0G2JFB8 | | | | |
| P26041 | Moesin | 7.29 ± 7.70 | 0.20 ± 0.42 | 97.25 |
| Q7TSG6 | | | | |
| P10107 | Annexin A1 | 7.25 ± 7.38 | 1.78 ± 2.11 | 75.48 |
| A0A494BBD8 | | | | |
| P40142 | Transketolase | 7.25 ± 4.68 | 2.44 ± 1.74 | 66.28 |
| A0A286YE28 | | | | |
| E0CY51 | | | | |
| P21614 | Vitamin D- | 7.25 ± 1.67 | 2.56 ± 0.73 | 64.75 |
| A0A0G2JGM6 | binding protein | | | |
| A9R9W0 | Major urinary | 7.00 ± 3.38 | 5.30 ± 2.06 | 24.29 |
| A2CEL1 | protein 1 | | | |
| P11588 | | | | |
| A2CEL0 | | | | |
| L7MUC7 | | | | |
| A2CEK9 | | | | |
| P26039 | Talin-1 | 6.88 ± 7.94 | 1.70 ± 2.45 | 75.27 |
| A2AIM2 | | | | |
| E9PUM4 | | | | |
| A0AILISQ51 | | | | |
| Q8CDM9 | | | | |
| Q71LX4 | | | | |
| F6SX70 | | | | |
| A0A1L1SRI1 | | | | |
| F6S1V7 | | | | |
| A0A1L1SQP9 | | | | |
| P68134 | Actin, alpha | 6.88 ± 3.09 | 4.30 ± 1.83 | 37.45 |
| P68033 | skeletal muscle | | | |
| P62806 | Histone H4 | 6.63 ± 3.78 | 4.50 ± 1.84 | 32.08 |
| P62737 | Actin, aortic | 6.50 ± 2.88 | 4.30 ± 1.83 | 33.85 |
| P63268 | smooth muscle | | | |
| A0A494B9T3 | | | | |
| D3YZY0 | | | | |
| D3Z2K3 | | | | |
| A0A0U1RQ96 | | | | |
| A0A0R4J0I1 | Serine protease | 6.38 ± 2.83 | 1.80 ± 0.92 | 71.76 |
| P07759 | inhibitor A3K | | | |
| Q80X76 | | | | |
| A0A2K6EDJ7 | Inter alpha- | 6.25 ± 1.58 | 2.30 ± 1.89 | 63.20 |
| E9Q5L2 | trypsin inhibitor, | | | |
| E9PVD2 | heavy chain 4 | | | |
| A6X935 | | | | |
| CON_Q3T052 | | | | |
| A0A0R4J0X5 | Alpha-1- | 6.25 ± 1.16 | 3.80 ± 0.79 | 39.20 |
| A0A0A0MQA3 | antitrypsin 1-3 | | | |
| Q00896 | | | | |
| P07758 | | | | |
| Q00897 | Alpha-1- | 6.25 ± 0.89 | 3.90 ± 0.99 | 37.60 |
| | antitrypsin 1-4 | | | |
| Q91VB8 | Hemoglobin | 6.13 ± 3.27 | 3.20 ± 2.44 | 47.76 |
| P01942 | subunit alpha | | | |
| P11247 | Myeloperoxidase | 6.13 ± 6.10 | 1.50 ± 2.32 | 75.51 |
| F7DC05 | | | | |
| F8WGZ9 | | | | |
| A0A0A0MQL9 | | | | |
| P10126 | Elongation factor | 6.13 ± 2.70 | 4.40 ± 3.06 | 28.16 |
| D3Z318 | 1-alpha 1 | | | |
| D3YZ68 | | | | |
| P62631 | | | | |
| P22599 | Alpha-1- | 6.13 ± 1.73 | 2.70 ± 0.67 | 55.92 |
| | antitrypsin 1-2 | | | |
| Q504P4 | Heat shock | 5.75 ± 4.13 | 3.13 ± 2.03 | 45.65 |
| P63017 | cognate 71 kDa | | | |
| D3Z5E2 | protein | | | |
| E9Q8I0 | Complement | 5.75 ± 1.04 | 1.22 ± 1.20 | 78.74 |
| P06909 | factor H | | | |
| D6RGQ0 | | | | |
| E9Q8H9 | | | | |
| A0A0A6YWP4 | | | | |
| A0A0A6YVP8 | | | | |
| A2CEK6 | Major urinary | 5.75 ± 2.92 | 3.60 ± 1.07 | 37.39 |
| B5X0G2 | protein 17 | | | |
| L7N222 | | | | |
| P58252 | Elongation factor | 5.63 ± 2.88 | 5.00 ± 4.58 | 11.11 |
| G3UXK8 | 2 | | | |
| G3UZ34 | | | | |
| A2AH85 | | | | |
| O08810 | | | | |
| Q9JKF1 | Ras GTPase- | 5.50 ± 6.35 | 0.44 ± 0.73 | 91.92 |
| A0A0UIRNG5 | activating-like | | | |
| Q3UQ44 | protein IQGAP1 | | | |
| A0A0U1RPU3 | | | | |
| Q3UQP1 | | | | |
| F8VQ29 | | | | |
| Q9D154 | Leukocyte | 5.38 ± 5.50 | 1.22 ± 1.99 | 77.26 |
| Z4YK03 | elastase inhibitor | | | |
| Q8VHP7 | A | | | |
| Q5SV42 | | | | |
| E9Q1Z0 | | 5.38 ± 1.41 | 3.10 ± 1.29 | 42.33 |
| P52480 | Pyruvate kinase | 5.25 ± 3.85 | 3.80 ± 2.49 | 27.62 |
| A0A1L1SU37 | PKM | | | |
| A0A1L1SQV8 | | | | |
| A0A1L1SUV0 | | | | |
| A0A1L1SSN6 | | | | |
| A0A1L1STV8 | | | | |
| A0A1L1ST52 | | | | |
| A0A1L1SVH2 | | | | |
| E9Q509 | | | | |
| G3X925 | | | | |
| P53657 | | | | |
| P23953 | Carboxylesterase | 5.25 ± 1.04 | 2.00 ± 0.82 | 61.90 |
| D3Z5G7 | 1C | | | |
| A0A1D5RM42 | | | | |
| Q8VCT4 | | | | |
| E9PYP1 | | | | |
| Q8VCC2 | | | | |
| P32261 | Antithrombin-III | 5.13 ± 2.42 | 1.70 ± 0.48 | 66.83 |
| A0A0A6YWH7 | | | | |
| CON_P41361 | | | | |
| A0A0A6YXS8 | | | | |
| A0A0A6YX49 | | | | |
| A0A0A6YX70 | | | | |
| P06728 | Apolipoprotein | 5.13 ± 1.64 | 1.60 ± 0.97 | 68.78 |
| | A-IV | | | |
| P01029 | Complement C4-B | 5.13 ± 2.42 | 0.00 ± 0.00 | 100.00 |
| P15864 | Histone H1.2 | 5.13 ± 2.17 | 4.20 ± 3.43 | 18.05 |
| P43277 | Histone H1.3 | | | |
| I7HFT9 | | | | |
| Q07133 | | | | |
| Q91X17 | Uromodulin | 5.00 ± 3.42 | 5.00 ± 2.06 | 0.00 |
| A0A140LI10 | | | | |
| P31725 | Protein S100-A9 | 5.00 ± 2.93 | 3.10 ± 2.18 | 38.00 |
| P17182 | Alpha-enolase | 5.00 ± 3.41 | 1.67 ± 1.87 | 66.67 |
| Q6PHC1 | | | | |
| B1ARR7 | | | | |
| B0QZL1 | | | | |
| A0A0N4SUI6 | | | | |
| B1ARR6 | | | | |
| Q5SX61 | | | | |
| Q5SX60 | | | | |
| D3YVD3 | | | | |
| D3Z2S4 | | | | |
| J3QPZ9 | | | | |
| Q5SX59 | | | | |
| D3Z6E4 | | | | |
| A0A0N4SUX5 | | | | |
| P17183 | | | | |
| Q61646 | Haptoglobin | 5.00 ± 2.14 | 2.33 ± 2.12 | 53.33 |
| P08226 | Apolipoprotein E | 4.88 ± 2.23 | 1.33 ± 0.50 | 72.65 |
| A0A1B0GX15 | | | | |
| G3UWN5 | | | | |
| G3UZM8 | | | | |
| D3YTY9 | Kininogen-1 | 4.88 ± 1.36 | 1.38 ± 1.19 | 71.79 |
| A0A0R4J038 | | | | |
| O08677 | | | | |
| D3Z2B2 | | | | |
| P05064 | Fructose- | 4.75 ± 2.19 | 2.67 ± 2.12 | 43.86 |
| A6ZI44 | bisphosphate | | | |
| Q9CPQ9 | aldolase A | | | |
| A6ZI46 | | | | |
| D3Z510 | | | | |
| A0A0U1RPN8 | | | | |
| D3YWI1 | | | | |
| D3YV98 | | | | |
| A0A0U1RPT5 | | | | |
| A6ZI47 | | | | |
| P07356 | Annexin A2 | 4.75 ± 4.06 | 1.78 ± 0.83 | 62.57 |
| B0V2N8 | | | | |
| B0V2N7 | | | | |
| B0V2N5 | | | | |
| P49290 | Eosinophil | 4.75 ± 5.57 | 0.00 ± 0.00 | 100.00 |
| | peroxidase | | | |
| O89053 | Coronin-1A | 4.75 ± 4.40 | 1.00 ± 1.80 | 78.95 |
| G3UYK8 | | | | |
| A0A0U1RPY8 | | | | |
| D3YW57 | | | | |
| G3UX53 | | | | |
| D3YXM2 | | | | |
| O35744 | Chitinase-like | 4.71 ± 4.19 | 1.30 ± 1.83 | 72.42 |
| F6RU51 | protein 3 | | | |
| Q06890 | Clusterin | 4.63 ± 1.19 | 2.30 ± 1.42 | 50.27 |
| E9PUU2 | | | | |
| E9PXG5 | | | | |
| E9Q8Y5 | | | | |
| E9Q9B8 | | | | |
| E9Q2G2 | | | | |
| Q8CBB6 | Histone H2B | 4.63 ± 1.85 | 3.60 ± 1.78 | 22.16 |
| Q8CGP2 | | | | |
| Q8CGP1 | | | | |
| Q6ZWY9 | | | | |
| Q64525 | | | | |
| Q64478 | | | | |
| Q64475 | | | | |
| P10854 | | | | |
| P10853 | | | | |
| P70696 | | | | |
| Q9D2U9 | | | | |
| Q8CGP0 | | | | |
| Q64524 | | | | |
| P99024 | Tubulin beta-5 | 4.63 ± 3.34 | 2.20 ± 1.93 | 52.43 |
| Q9CWF2 | chain | | | |
| Q7TMM9 | | | | |
| P68372 | | | | |
| Q9D6F9 | | | | |
| Q922F4 | | | | |
| Q9ERD7 | | | | |
| CON_ENSEMB | | | | |
| L:ENSBTAP0000 | | | | |
| O025008 | | | | |
| A2AQ07 | | | | |
| G3UZR1 | | | | |
| A0A1D5RM76 | | | | |
| P45376 | Aldose reductase | 4.63 ± 1.69 | 4.50 ± 2.92 | 2.70 |
| D3YVJ7 | | | | |
| P06745 | Glucose-6- | 4.57 ± 4.28 | 1.50 ± 2.12 | 67.19 |
| A0A0UIRQ72 | phosphate | | | |
| A0A0UIRP97 | isomerase | | | |
| CON_Q3ZBD7 | | | | |
| A0A0UIRQ18 | | | | |
| O08692 | Neutrophilic | 4.38 ± 2.39 | 2.20 ± 1.62 | 49.71 |
| | granule protein | | | |
| O35639 | Annexin A3 | 4.33 ± 3.20 | 1.56 ± 1.67 | 64.10 |
| Q3TET3 | | | | |
| A0A0G2JDV9 | | | | |
| A0A0G2JGL7 | | | | |
| Q9ET01 | Glycogen | 4.25 ± 5.26 | 0.75 ± 1.04 | 82.35 |
| Q3UEJ6 | phosphorylase, | | | |
| E9PUM3 | liver form | | | |
| | Alpha-1,4 glucan | | | |
| | phosphorylase | | | |
| P43274 | Histone H1.4 | 4.25 ± 2.25 | 3.10 ± 2.28 | 27.06 |
| P11499 | Heat shock | 4.14 ± 2.27 | 2.60 ± 3.20 | 37.24 |
| E9PX27 | protein HSP 90- | | | |
| E9Q3D6 | beta | | | |
| D3Z1R1 | | | | |
| E9Q0C3 | | | | |
| A2A513 | Keratin, type I | 4.13 ± 2.64 | 1.80 ± 0.92 | 56.36 |
| CON_P02535-1 | cytoskeletal 10 | | | |
| P02535 | | | | |
| A0A0R4J039 | Histidine-rich | 4.00 ± 1.77 | 1.22 ± 0.97 | 69.44 |
| Q9ESB3 | glycoprotein | | | |
| A0A338P6H8 | | | | |
| D3Z6F5 | ATP synthase | 3.88 ± 2.75 | 2.78 ± 2.05 | 28.32 |
| Q03265 | subunit alpha | | | |
| Q831A3 | | | | |
| S4R1W1 | Glyceraldehyde- | 3.88 ± 1.96 | 2.33 ± 1.41 | 39.78 |
| P16858 | 3-phosphate | | | |
| A0A1D5RLD8 | dehydrogenase | | | |
| A0A0A0MQF6 | | | | |
| S4R257 | | | | |
| S4R1W8 | | | | |
| V9GX06 | | | | |
| A0A0R4J0X7 | | | | |
| S4R2G5 | | | | |
| Q64467 | | | | |
| S4RIN5 | | | | |
| V9GXK0 | | | | |
| A0A1B0GSR9 | L-lactate | 3.88 ± 1.81 | 2.30 ± 1.16 | 40.65 |
| A0A1B0GSX0 | dehydrogenase A | | | |
| P06151 | chain | | | |
| A0A1B0GSL7 | | | | |
| A0A1B0GT41 | | | | |
| A0A1B0GQX5 | | | | |
| D3YZQ9 | | | | |
| A0A1B0GRW9 | | | | |
| A0A1B0GS79 | | | | |
| A0A1B0GRC1 | | | | |
| A0A1B0GRS2 | | | | |
| A0A1B0GSR2 | | | | |
| D3YVR7 | | | | |
| D3YZE4 | | | | |
| A0A1B0GRE9 | | | | |
| P00342 | | | | |
| B8JJM5 | Complement | 3.88 ± 1.64 | 0.56 ± 0.53 | 85.66 |
| F6VQX8 | factor B | | | |
| B8JJN0 | | | | |
| P04186 | | | | |
| F6XQ00 | | | | |
| B8JJM6 | | | | |
| H3BK95 | | | | |
| B8JJM3 | | | | |
| F6W2T4 | | | | |
| P63101 | 14-3-3 protein | 3.88 ± 2.59 | 3.90 ± 2.69 | −0.65 |
| A0A2I3BQ03 | zeta/delta | | | |
| D3YXN6 | | | | |
| D3YXF4 | | | | |
| D3YW45 | | | | |
| P26040 | Ezrin | 3.86 ± 3.80 | 2.00 ± 2.79 | 48.15 |
| Q62266 | Cornifin-A | 3.75 ± 1.98 | 4.30 ± 2.50 | −14.67 |
| Q62267 | | | | |
| P68373 | Tubulin alpha-1C | 3.75 ± 1.39 | 2.78 ± 2.05 | 25.93 |
| P05213 | chain | | | |
| A0A2R8VHF3 | | | | |
| Q9JJZ2 | | | | |
| Q3UX10 | | | | |
| P24527 | Leukotriene A-4 | 3.75 ± 3.81 | 0.80 ± 1.14 | 78.67 |
| | hydrolase | | | |
| P19221 | Prothrombin | 3.75 ± 2.25 | 1.00 ± 1.22 | 73.33 |
| H7BX99 | | | | |
| CON_P00735 | | | | |
| P04104 | Keratin, type II | 3.75 ± 2.76 | 2.00 ± 1.00 | 46.67 |
| | cytoskeletal 1 | | | |
| Q00898 | Alpha-1- | 3.75 ± 1.16 | 1.80 ± 0.63 | 52.00 |
| | antitrypsin 1-5 | | | |
| O70456 | 14-3-3 protein | 3.71 ± 1.70 | 5.10 ± 2.56 | −37.31 |
| | sigma | | | |
| A0A0N4SV66 | Histone H2A | 3.71 ± 1.70 | 2.00 ± 1.00 | 46.15 |
| Q8CGP4 | | | | |
| C0HKE9 | | | | |
| C0HKE8 | | | | |
| C0HKE7 | | | | |
| C0HKE6 | | | | |
| C0HKE5 | | | | |
| C0HKE4 | | | | |
| C0HKE3 | | | | |
| C0HKE2 | | | | |
| C0HKE1 | | | | |
| Q8CGP6 | | | | |
| Q8R1M2 | | | | |
| Q8CGP7 | | | | |
| Q8CGP5 | | | | |
| Q8BFU2 | | | | |
| Q64523 | | | | |
| Q6GSS7 | | | | |
| P27661 | | | | |
| Q64522 | | | | |
| G3UWL7 | | | | |
| P07901 | Heat shock | 3.71 ± 2.43 | 1.20 ± 1.32 | 67.69 |
| B7ZC50 | protein HSP 90- | | | |
| A2A6A2 | alpha | | | |
| B7ZC49 | | | | |
| REV_Q8BL66 | | | | |
| A0A0G2JEU1 | Aldehyde | 3.63 ± 3.74 | 0.30 ± 0.67 | 91.72 |
| P47738 | dehydrogenase, | | | |
| A0A0G2JF60 | mitochondrial | | | |
| A0A0G2JFQ0 | | | | |
| D3YYF3 | | | | |
| Q62148 | | | | |
| G3UWP3 | | | | |
| P24549 | | | | |
| Q9JHW9 | | | | |
| Q9CZS1 | | | | |
| A0A1W2P768 | Histone H3.2 | 3.63 ± 2.39 | 1.70 ± 1.34 | 53.10 |
| P84228 | | | | |
| P68433 | | | | |
| F8WI35 | | | | |
| P84244 | | | | |
| P02301 | | | | |
| E0CZ27 | | | | |
| E0CYN1 | | | | |
| E0CYR7 | | | | |
| P07309 | Transthyretin | 3.63 ± 0.52 | 1.80 ± 0.79 | 50.34 |
| P68369 | Tubulin alpha-1A | 3.63 ± 1.30 | 2.56 ± 1.67 | 29.50 |
| P05214 | chain | | | |
| O88342 | WD repeat- | 3.50 ± 3.66 | 2.10 ± 1.91 | 40.00 |
| A0A0J9YU05 | containing | | | |
| | protein 1 | | | |
| Q3UV17 | Keratin, type II | 3.50 ± 1.07 | 2.30 ± 0.82 | 34.29 |
| CON_Q7RTS7 | cytoskeletal 2 | | | |
| CON_Q32MB2 | oral | | | |
| O88569 | Heterogeneous | 3.43 ± 4.20 | 0.67 ± 0.71 | 80.56 |
| A0A0N4SUM2 | nuclear | | | |
| | ribonucleoproteins | | | |
| | A2/B1 | | | |
| A1BN54 | Alpha-actinin-1 | 3.38 ± 5.15 | 0.00 ± 0.00 | 100.00 |
| Q7TPR4 | | | | |
| P17751 | Triosephosphate | 3.38 ± 2.39 | 2.00 ± 1.50 | 40.74 |
| H7BXC3 | isomerase | | | |
| Q00612 | Glucose-6- | 3.38 ± 3.42 | 1.00 ± 1.25 | 70.37 |
| A3KG36 | phosphate 1- | | | |
| Q836V0 | dehydrogenase X | | | |
| G3UWD6 | | | | |
| REV_P46662 | | | | |
| P97324 | | | | |
| Q9DCD0 | 6-phosphogluconate | 3.38 ± 2.92 | 1.56 ± 1.74 | 53.91 |
| | dehydrogenase, | | | |
| | decarboxylating | | | |
| Q03734 | Serine protease | 3.38 ± 1.60 | 1.00 ± 0.71 | 70.37 |
| P29621 | inhibitor A3M | | | |
| O88844 | Isocitrate | 3.33 ± 1.75 | 2.90 ± 2.18 | 13.00 |
| A0A087WPT4 | dehydrogenase | | | |
| A0A087WRS9 | [NADP] | | | |
| D3YVY3 | cytoplasmic | | | |
| A0A087WRM4 | | | | |
| A0A0UIRP68 | | | | |
| D6RIL6 | | | | |
| P54071 | | | | |
| P27773 | Protein disulfide- | 3.33 ± 2.42 | 1.00 ± 1.00 | 70.00 |
| F6Q404 | isomerase A3 | | | |
| A0A1B0GR11 | Transaldolase | 3.29 ± 2.87 | 0.78 ± 0.97 | 76.33 |
| Q93092 | | | | |
| F8WGL3 | Cofilin-1 | 3.29 ± 1.98 | 1.33 ± 0.50 | 59.42 |
| P18760 | | | | |
| A0A494B9A7 | | | | |
| Q3THW5 | Histone H2A.V | 3.29 ± 1.50 | 1.44 ± 0.53 | 56.04 |
| P0C0S6 | | | | |
| Q8R029 | | | | |
| Q3UA95 | | | | |
| Q542I8 | Integrin beta | 3.25 ± 3.01 | 0.90 ± 1.91 | 72.31 |
| P11835 | | | | |
| M0QWA7 | | | | |
| D3YYP8 | | | | |
| D3Z1S4 | | | | |
| P29788 | Vitronectin | 3.25 ± 1.49 | 1.00 ± 1.12 | 69.23 |
| Q3UP87 | Neutrophil | 3.14 ± 2.48 | 0.90 ± 1.20 | 71.36 |
| | elastase | | | |
| Q9CQV8 | 14-3-3 protein | 3.14 ± 1.35 | 2.00 ± 1.41 | 36.36 |
| A2A5N1 | beta/alpha | | | |
| E9Q604 | Integrin alpha-M | 3.13 ± 4.52 | 0.11 ± 0.33 | 96.44 |
| G5E8F1 | | | | |
| E9Q5K8 | | | | |
| Q3U1U4 | | | | |
| P05555 | | | | |
| A0A0R4J1B4 | | | | |
| P29699 | Alpha-2-HS- | 3.13 ± 1.73 | 0.67 ± 0.71 | 78.67 |
| A0A338P703 | glycoprotein | | | |
| A0A338P7G1 | | | | |
| A0A338P7H5 | | | | |
| A0A338P692 | | | | |
| A0A338P6Y6 | | | | |
| CON_P12763 | | | | |
| P08752 | Guanine | 3.13 ± 3.36 | 0.33 ± 0.50 | 89.33 |
| A0A0A6YWA9 | nucleotide- | | | |
| B2RSH2 | binding protein | | | |
| A2AE32 | G(i) subunit | | | |
| P20612 | alpha-2 | | | |
| P18872 | | | | |
| Q3V3I2 | | | | |
| P50149 | | | | |
| A2A610 | | | | |
| A2AE31 | | | | |
| Q8C040 | | | | |
| D3Z2M7 | | | | |
| Q8BHK8 | | | | |
| F6QPU5 | | | | |
| Z4YKV1 | | | | |
| Q66L47 | | | | |
| Q8CGK7 | | | | |
| P63094 | | | | |
| Q6R0H7 | | | | |
| P24472 | Glutathione S- | 3.13 ± 0.83 | 4.70 ± 1.70 | −50.40 |
| A0A1L1SS61 | transferase A4 | | | |
| P97872 | Dimethylaniline | 3.00 ± 1.85 | 4.22 ± 3.03 | −40.74 |
| Q14DT3 | monooxygenase | | | |
| Q8C116 | [N-oxide- | | | |
| | forming] 5 | | | |
| Q01339 | Beta-2- | 3.00 ± 1.41 | 0.25 ± 0.46 | 91.67 |
| I7HJR3 | glycoprotein 1 | | | |
| CON_P17690 | | | | |
| P04117 | Fatty acid- | 3.00 ± 1.41 | 3.22 ± 1.72 | −7.41 |
| A0A0A6YW05 | binding protein, | | | |
| A0A0A6YXB9 | adipocyte | | | |
| A0A0A6YXI2 | | | | |
| P24526 | | | | |
| O08716 | | | | |
| Q9CVB6 | Actin-related | 3.00 ± 2.78 | 1.00 ± 0.82 | 66.67 |
| D3YXG6 | protein 2/3 | | | |
| A0A087WRT2 | complex | | | |
| | subunit 2 | | | |
| Q05144 | Ras-related C3 | 3.00 ± 1.69 | 1.00 ± 1.00 | 66.67 |
| A0A2R8VHH0 | botulinum toxin | | | |
| | substrate 2 | | | |
| D3Z6I8 | Tropomyosin | 3.00 ± 3.34 | 0.25 ± 0.46 | 91.67 |
| E9Q7Q3 | alpha-3 chain | | | |
| A0A0R4J1P2 | | | | |
| P21107 | | | | |
| D3YVR0 | | | | |
| A2AIM5 | | | | |
| S4R2U0 | | | | |
| G5E8R0 | | | | |
| E9Q453 | | | | |
| G5E8R2 | | | | |
| G5E8R1 | | | | |
| E9Q456 | | | | |
| E9Q455 | | | | |
| E9Q452 | | | | |
| Q8BSH3 | | | | |
| Q8BP43 | | | | |
| E9Q450 | | | | |
| E9Q448 | | | | |
| CON_Q3SX28 | | | | |
| B7ZNL3 | | | | |
| A2AIM4 | | | | |
| E9Q454 | | | | |
| F8WID5 | | | | |
| P58774 | | | | |
| P58771 | | | | |
| E9Q3Z4 | Hexokinase | 2.88 ± 4.22 | 0.11 ± 0.33 | 96.14 |
| Q3TRM8 | | | | |
| E9Q8S8 | | | | |
| D6RFA3 | | | | |
| P52792 | | | | |
| P09411 | Phosphoglycerate | 2.88 ± 3.72 | 0.67 ± 0.71 | 76.81 |
| S4R2M7 | kinase 1 | | | |
| P09041 | | | | |
| D3Z2H9 | | 2.88 ± 3.27 | 0.11 ± 0.33 | 96.14 |
| E9Q5J9 | | | | |
| A0A0A0MQA5 | Tubulin alpha-4A | 2.88 ± 2.64 | 1.70 ± 1.95 | 40.87 |
| P68368 | chain | | | |
| A0A087WQS4 | | | | |
| A0A087WRB4 | | | | |
| A0A087WSB0 | | | | |
| A0A087WSL5 | | | | |
| P51881 | ADP/ATP | 2.86 ± 1.77 | 1.00 ± 0.94 | 65.00 |
| P48962 | translocase 2 | | | |
| Q3V132 | | | | |
| A0A2R8VHF9 | Myosin-11 | 2.83 ± 2.23 | 0.67 ± 0.71 | 76.47 |
| E9QPE7 | | | | |
| A0A338P6K2 | | | | |
| O08638 | | | | |
| Q5SXR6 | Clathrin heavy | 2.75 ± 4.03 | 0.44 ± 0.73 | 83.84 |
| Q68FD5 | chain | | | |
| F6Z1R4 | | | | |
| Q61598 | Rab GDP | 2.75 ± 3.06 | 1.00 ± 1.41 | 63.64 |
| A0A1Y7VL99 | dissociation | | | |
| A0A1Y7VLG4 | inhibitor beta | | | |
| P08113 | Endoplasmin | 2.75 ± 3.20 | 0.80 ± 1.14 | 70.91 |
| F7C312 | | | | |
| Q8C253 | Galectin | 2.75 ± 1.98 | 0.78 ± 0.83 | 71.72 |
| P16110 | | | | |
| P43276 | Histone H1.5 | 2.75 ± 1.39 | 1.10 ± 1.10 | 60.00 |
| H3BJQ7 | Peroxiredoxin-5, | 2.71 ± 1.50 | 1.50 ± 0.53 | 44.74 |
| P99029 | mitochondrial | | | |
| A0A494BAZ4 | | | | |
| G3UZJ4 | | | | |
| P40124 | Adenylyl | 2.63 ± 2.72 | 1.78 ± 1.09 | 32.28 |
| B1ARS0 | cyclase- | | | |
| D3YTR7 | associated protein | | | |
| A0A286YCS6 | 1 | | | |
| Q9CYT6 | | | | |
| P56480 | ATP synthase | 2.63 ± 1.92 | 1.50 ± 2.12 | 42.86 |
| Q831A5 | subunit beta, | | | |
| | mitochondrial | | | |
| P48036 | Annexin A5 | 2.63 ± 2.33 | 0.88 ± 0.64 | 66.67 |
| A0A0G2JGQ0 | | | | |
| Q99JI6 | Ras-related | 2.57 ± 2.57 | 0.80 ± 1.23 | 68.89 |
| A0A0G2JDL9 | protein Rap-1b | | | |
| P62835 | | | | |
| A0A0G2JE52 | | | | |
| A0A0G2JED9 | | | | |
| A0A1W2P777 | | | | |
| P62962 | Profilin-1 | 2.57 ± 2.37 | 0.78 ± 0.83 | 69.75 |
| Q5SX49 | | | | |
| CON_P02584 | | | | |
| Q9R0P5 | Destrin | 2.50 ± 1.41 | 1.56 ± 1.33 | 37.78 |
| P45591 | | | | |
| P15947 | Kallikrein-1 | 2.50 ± 2.62 | 1.90 ± 0.99 | 24.00 |
| A0A0U1RPN5 | | | | |
| P15945 | | | | |
| P00757 | | | | |
| Q61759 | | | | |
| P15946 | | | | |
| Q5FW60 | Major urinary | 2.50 ± 2.20 | 1.00 ± 0.94 | 60.00 |
| | protein 20 | | | |
| P01898 | H-2 class I | 2.50 ± 1.20 | 0.00 ± 0.00 | 100.00 |
| A0A494B9G2 | histocompatibility | | | |
| A0A0B4J1G3 | antigen, Q10 | | | |
| A0A494B9G8 | alpha chain | | | |
| E9PWT4 | | | | |
| E9QJR9 | | | | |
| P79568 | | | | |
| E9PX63 | | | | |
| Q8HWB2 | | | | |
| A0A494BA33 | | | | |
| A0A494BAT0 | | | | |
| Q3TH01 | | | | |
| O19441 | | | | |
| A7VMS6 | | | | |
| E9Q0G4 | | | | |
| G3UXE9 | | | | |
| P01895 | | | | |
| P14430 | | | | |
| P14428 | | | | |
| P14429 | | | | |
| P14426 | | | | |
| P01900 | | | | |
| P03991 | | | | |
| P04223 | | | | |
| P01901 | | | | |
| Q9DBJ1 | Phosphoglycerate | 2.43 ± 2.15 | 0.50 ± 0.53 | 79.41 |
| O70250 | mutase 1 | | | |
| A0A075B5P6 | Ig mu chain C | 2.43 ± 1.90 | 0.00 ± 0.00 | 100.00 |
| A0A075B6A0 | region | | | |
| P01872 | | | | |
| Q01853 | Transitional | 2.38 ± 2.20 | 1.60 ± 1.65 | 32.63 |
| | endoplasmic | | | |
| | reticulum | | | |
| | ATPase | | | |
| Q3KQQ2 | Major urinary | 2.38 ± 2.07 | 1.40 ± 1.07 | 41.05 |
| P04939 | protein 3 | | | |
| Q80YX8 | | | | |
| P11590 | | | | |
| P17156 | Heat shock- | 2.38 ± 1.60 | 1.38 ± 0.74 | 42.11 |
| | related 70 kDa | | | |
| | protein 2 | | | |
| P20029 | 78 kDa glucose- | 2.29 ± 2.36 | 0.40 ± 0.84 | 82.50 |
| | regulated protein | | | |
| P08249 | Malate | 2.29 ± 1.98 | 1.20 ± 1.14 | 47.50 |
| A0A0G2JF23 | dehydrogenase, | | | |
| A0A0G2JGY4 | mitochondrial | | | |
| P43275 | Histone H1.1 | 2.29 ± 1.25 | 1.22 ± 1.30 | 46.53 |
| P00920 | Carbonic | 2.25 ± 2.66 | 0.75 ± 0.89 | 66.67 |
| A0A0A6YX78 | anhydrase 2 | | | |
| P30681 | High mobility | 2.25 ± 2.55 | 0.00 ± 0.00 | 100.00 |
| A0A1B0GQX9 | group protein B2 | | | |
| O89020 | Afamin | 2.25 ± 1.16 | 0.00 ± 0.00 | 100.00 |
| Q6S9I0 | | 2.25 ± 0.71 | 0.75 ± 0.46 | 66.67 |
| Q6S9I2 | | | | |
| Q6S9I3 | | | | |
| A0A338P699 | | | | |
| A0A338P7D0 | | | | |
| E0CYI5 | | | | |
| CON_P01045-1 | | | | |
| CON_Q2KJ62 | | | | |
| CON_P01044-1 | | | | |
| P16125 | L-lactate | 2.17 ± 0.75 | 1.78 ± 1.72 | 17.95 |
| A0A0N4SVV8 | dehydrogenase B | | | |
| D3Z7F0 | chain | | | |
| P14733 | Lamin-B1 | 2.14 ± 2.48 | 0.44 ± 0.73 | 79.26 |
| A0A0R4J0Q5 | | | | |
| P21619 | | | | |
| B1AXW5 | Peroxiredoxin-1 | 2.14 ± 0.90 | 2.10 ± 1.20 | 2.00 |
| B1AXW6 | | | | |
| P35700 | | | | |
| B1AXW4 | | | | |
| B1AZS9 | | | | |
| O08807 | | | | |
| P04919 | Band 3 anion | 2.13 ± 3.36 | 0.80 ± 1.69 | 62.35 |
| | transport protein | | | |
| Q3UDS7 | ADP-dependent | 2.13 ± 2.47 | 0.30 ± 0.67 | 85.88 |
| A0A1L1SSF2 | glucokinase | | | |
| Q8VDL4 | | | | |
| A0A075B5P3 | Ig gamma-2B | 2.13 ± 1.13 | 0.11 ± 0.33 | 94.77 |
| A0A0A6YVP0 | chain C region | | | |
| P01867 | | | | |
| P17742 | Peptidyl-prolyl | 2.13 ± 1.13 | 1.00 ± 1.22 | 52.94 |
| A0A1L1SST0 | cis-trans | | | |
| V9GXC1 | isomerase A | | | |
| V9GX31 | | | | |
| P16045 | Galectin-1 | 2.13 ± 0.64 | 0.00 ± 0.00 | 100.00 |
| A0A2R8VHP3 | | 2.13 ± 0.99 | 1.40 ± 0.97 | 34.12 |
| Q6IFZ8 | | | | |
| D3Z6R0 | | | | |
| Q64727 | Vinculin | 2.00 ± 2.20 | 0.80 ± 1.32 | 60.00 |
| P42932 | T-complex | 2.00 ± 2.08 | 1.22 ± 1.48 | 38.89 |
| H3BL49 | protein 1 subunit | | | |
| H3BJB6 | theta | | | |
| H3BKR8 | | | | |
| H3BLL1 | | | | |
| H3BKG2 | | | | |
| Q61129 | Complement | 2.00 ± 1.60 | 0.00 ± 0.00 | 100.00 |
| A0A0G2JF07 | factor I | | | |
| P09528 | Ferritin heavy | 2.00 ± 2.45 | 0.00 ± 0.00 | 100.00 |
| A0A494BA92 | chain | | | |
| A0A494B9D4 | | | | |
| A0A494BAP3 | | | | |
| H3BKI8 | Heterogeneous | 2.00 ± 2.16 | 0.40 ± 0.52 | 80.00 |
| A0A286YDM3 | nuclear | | | |
| H3BK96 | ribonucleoprotein | | | |
| H3BKD0 | K | | | |
| B2M1R6 | | | | |
| P61979 | | | | |
| A0A286YCM2 | | | | |
| H3BLL4 | | | | |
| A0A286YEC4 | | | | |
| A0A286YE41 | | | | |
| H3BJ43 | | | | |
| H3BJS9 | | | | |
| Q8BT23 | | | | |
| A0A286YDH1 | | | | |
| P20065 | Thymosin beta-4 | 2.00 ± 1.07 | 1.56 ± 1.24 | 22.22 |
| Q19LI2 | Alpha-1B- | 2.00 ± 1.73 | 0.00 ± 0.00 | 100.00 |
| | glycoprotein | | | |
| Q9QUI0 | Transforming | 2.00 ± 1.83 | 0.70 ± 1.06 | 65.00 |
| A0A0A6YXF6 | protein RhoA | | | |
| Q62159 | | | | |
| A0A0G2JEP8 | | | | |
| H3BL56 | | | | |
| A0A0A6YWJ1 | | | | |
| Q9CR99 | | | | |
| P62746 | | | | |
| E9PZF0 | Nucleoside | 2.00 ± 1.41 | 0.30 ± 0.67 | 85.00 |
| Q01768 | diphosphate | | | |
| Q5NC79 | kinase | | | |
| Q07456 | Protein AMBP | 2.00 ± 1.31 | 0.00 ± 0.00 | 100.00 |
| P16015 | Carbonic | 2.00 ± 0.93 | 0.60 ± 0.52 | 70.00 |
| | anhydrase 3 | | | |
| Q9Z1Q5 | Chloride | 2.00 ± 1.63 | 0.30 ± 0.67 | 85.00 |
| | intracellular | | | |
| | channel protein 1 | | | |
| Q3TLP8 | Ras-related C3 | 2.00 ± 1.20 | 0.78 ± 0.83 | 61.11 |
| P63001 | botulinum toxin | | | |
| P60764 | substrate 1 | | | |
| A2AC13 | | | | |
| A0A2R8VH29 | | | | |
| A0A1B0GSL4 | | | | |
| G3UZM2 | | | | |
| D3Z3L1 | | | | |
| F2Z463 | | | | |
| D3YX61 | | | | |
| Q8R527 | | | | |
| Q9ER71 | | | | |
| O09131 | Glutathione S- | 1.88 ± 0.64 | 2.13 ± 1.81 | −13.33 |
| A0A494BAB1 | transferase | | | |
| A0A494B9X6 | omega-1 | | | |
| A0A494BAY2 | | | | |
| A0A494BB82 | | | | |
| Q8K2Q2 | | | | |
| F8WIT2 | Annexin | 1.88 ± 2.90 | 0.00 ± 0.00 | 100.00 |
| P14824 | | | | |
| P47791 | Glutathione | 1.88 ± 2.10 | 0.80 ± 1.14 | 57.33 |
| | reductase, | | | |
| | mitochondrial | | | |
| Q8BND5 | Sulfhydryl | 1.88 ± 1.73 | 0.00 ± 0.00 | 100.00 |
| | oxidase 1 | | | |
| D3YY36 | Inhibitor of | 1.88 ± 0.99 | 0.43 ± 0.53 | 77.14 |
| Q9DBD0 | carbonic | | | |
| F6W4D3 | anhydrase | | | |
| E9PWU4 | Adiponectin | 1.88 ± 0.35 | 0.11 ± 0.33 | 94.07 |
| Q60994 | | | | |
| P61982 | 14-3-3 protein | 1.88 ± 1.25 | 1.70 ± 1.42 | 9.33 |
| | gamma | | | |
| Q9DC51 | Guanine | 1.88 ± 2.03 | 0.33 ± 0.50 | 82.22 |
| | nucleotide- | | | |
| | binding protein | | | |
| | G(k) subunit | | | |
| | alpha | | | |
| F8WJ05 | Inter-alpha- | 1.86 ± 0.69 | 0.00 ± 0.00 | 100.00 |
| Q61702 | trypsin inhibitor | | | |
| | heavy chain H1 | | | |
| P08228 | Superoxide | 1.83 ± 0.75 | 1.89 ± 1.54 | −3.03 |
| | dismutase | | | |
| | [Cu—Zn] | | | |
| Q9Z2U0 | Proteasome | 1.83 ± 0.98 | 0.89 ± 1.05 | 51.52 |
| A0A338P7D7 | subunit alpha | | | |
| Q9CWH6 | type-7 | | | |
| B7ZMS4 | | | | |
| Q8BJS4 | SUN domain- | 1.75 ± 1.98 | 0.20 ± 0.42 | 88.57 |
| | containing | | | |
| | protein 2 | | | |
| Q5SW88 | Ras-related | 1.75 ± 1.49 | 1.13 ± 1.25 | 35.71 |
| P62821 | protein Rab-1A | | | |
| Q5SW87 | | | | |
| A0A494BA38 | | | | |
| A0A494BBL7 | | | | |
| A0A494B945 | | | | |
| Q9D1G1 | | | | |
| Q5SW86 | | | | |
| Q9DBB9 | Carboxypeptidase | 1.75 ± 0.46 | 0.00 ± 0.00 | 100.00 |
| | N subunit 2 | | | |
| P51150 | Ras-related | 1.75 ± 1.91 | 0.44 ± 0.73 | 74.60 |
| A0A0N4SVR6 | protein Rab-7a | | | |
| A0A0N4SVG9 | | | | |
| E9Q1Y9 | Keratin, type II | 1.75 ± 1.39 | 0.75 ± 0.46 | 57.14 |
| CON_Q61726 | cuticular Hb1 | | | |
| Q9ERE2 | | | | |
| P97861 | | | | |
| Q6IMF0 | | | | |
| CON_P78386 | | | | |
| Q9Z2T6 | | | | |
| CON_043790 | | | | |
| CON_Q6NT21 | | | | |
| CON_P78385 | | | | |
| CON_Q14533 | | | | |
| A0A0A6YW67 | Ubiquitin-60S | 1.75 ± 0.89 | 1.50 ± 0.53 | 14.29 |
| E9Q9J0 | ribosomal protein | | | |
| E9Q4P0 | L40 | | | |
| E9Q5F6 | | | | |
| E9QNP0 | | | | |
| Q5SX22 | | | | |
| P62984 | | | | |
| P62983 | | | | |
| P0CG49 | | | | |
| P0CG50 | | | | |
| D3YX76 | Glutathione S- | 1.75 ± 0.46 | 1.40 ± 0.70 | 20.00 |
| P15626 | transferase Mu 2 | | | |
| P47911 | 60S ribosomal | 1.71 ± 1.98 | 1.56 ± 1.59 | 9.26 |
| A0A0J9YU32 | protein L6 | | | |
| P08905 | Lysozyme C-2 | 1.71 ± 1.38 | 0.70 ± 1.06 | 59.17 |
| P17897 | | | | |
| Q9CQI6 | Coactosin-like | 1.71 ± 1.38 | 0.50 ± 0.71 | 70.83 |
| A0A1D5RLP1 | protein | | | |
| Q9CZX8 | 40S ribosomal | 1.67 ± 1.03 | 1.10 ± 0.99 | 34.00 |
| D3YUT3 | protein S19 | | | |
| D3YUG3 | | | | |
| D3Z5R8 | | | | |
| D3Z722 | | | | |
| S4R223 | | | | |
| P14069 | Protein S100-A6 | 1.63 ± 0.52 | 2.00 ± 0.47 | −23.08 |
| Q8BH61 | Coagulation | 1.63 ± 2.20 | 0.00 ± 0.00 | 100.00 |
| | factor XIII A | | | |
| | chain | | | |
| P60843 | Eukaryotic | 1.63 ± 0.92 | 1.67 ± 1.32 | −2.56 |
| Q8BTU6 | initiation factor | | | |
| P10630 | 4A-I | | | |
| E9Q561 | | | | |
| A0A338P6X5 | | | | |
| P48999 | Arachidonate 5- | 1.63 ± 2.07 | 0.11 ± 0.33 | 93.16 |
| E9QA93 | lipoxygenase | | | |
| E9Q6H6 | | | | |
| A0A0N4SW45 | | | | |
| A0A1B0GSG5 | Ribonuclease | 1.63 ± 1.30 | 0.90 ± 1.20 | 44.62 |
| Q91VI7 | inhibitor | | | |
| A0A1B0GRG4 | | | | |
| S4RIN6 | 40S ribosomal | 1.63 ± 0.92 | 0.78 ± 0.83 | 52.14 |
| F6YVP7 | protein S18 | | | |
| P62270 | | | | |
| A0A1Y7VKY1 | | | | |
| A0A3Q4EGP3 | | | | |
| Q5ND35 | Alpha-2- | 1.63 ± 1.41 | 0.00 ± 0.00 | 100.00 |
| Q61247 | antiplasmin | | | |
| E9PXE0 | | | | |
| Q99JY9 | Actin-related | 1.63 ± 1.77 | 0.50 ± 0.71 | 69.23 |
| A0A087WS98 | protein 3 | | | |
| A0A087WP86 | | | | |
| A0A087WQ14 | | | | |
| A0A087WRA1 | | | | |
| Q641P0 | | | | |
| A0A087WQ83 | | | | |
| P97369 | Neutrophil | 1.63 ± 1.85 | 0.11 ± 0.33 | 93.16 |
| A8XU21 | cytosol factor 4 | | | |
| Q8BT60 | Copine-3 | 1.63 ± 1.77 | 0.00 ± 0.00 | 100.00 |
| Q9D6C8 | | | | |
| A0A0R4J0J1 | | | | |
| Q3UYN2 | | | | |
| Q1RLL3 | | | | |
| Q9Z140 | | | | |
| Q8BLR2 | | | | |
| Q0VE82 | | | | |
| Q9DC53 | | | | |
| Q8JZW4 | | | | |
| P09405 | Nucleolin | 1.57 ± 1.72 | 1.20 ± 1.40 | 23.64 |
| Q91YR9 | Prostaglandin | 1.57 ± 1.27 | 0.60 ± 0.84 | 61.82 |
| REV_Q3TVC7 | reductase 1 | | | |
| REV_A0A0R4J177 | | | | |
| P50247 | Adenosylhomocy | 1.57 ± 0.98 | 1.00 ± 0.94 | 36.36 |
| A2ALT5 | steinase | | | |
| P05202 | Aspartate | 1.57 ± 1.27 | 0.70 ± 1.06 | 55.45 |
| | aminotransferase, | | | |
| | mitochondrial | | | |
| P62259 | 14-3-3 protein | 1.57 ± 1.13 | 1.89 ± 0.93 | −20.20 |
| F6WA09 | epsilon | | | |
| D6REF3 | | | | |
| P84096 | Rho-related GTP- | 1.57 ± 0.79 | 0.56 ± 0.53 | 64.65 |
| | binding protein | | | |
| | RhoG | | | |
| P62908 | 40S ribosomal | 1.50 ± 1.87 | 1.56 ± 1.74 | −3.70 |
| D3YV43 | protein S3 | | | |
| A0A140LI77 | | | | |
| P97384 | Annexin A11 | 1.50 ± 2.00 | 0.25 ± 0.46 | 83.33 |
| D3Z7U0 | | | | |
| O70145 | Neutrophil | 1.50 ± 1.93 | 0.00 ± 0.00 | 100.00 |
| A0A087WPH0 | cytosol factor 2 | | | |
| Q543K9 | Purine nucleoside | 1.50 ± 1.85 | 0.33 ± 0.50 | 77.78 |
| P23492 | phosphorylase | | | |
| A0A2I3BQH2 | | | | |
| A0A2I3BS22 | | | | |
| Q9D8C9 | | | | |
| A0A0J9YUZ4 | High mobility | 1.50 ± 1.69 | 0.00 ± 0.00 | 100.00 |
| P63158 | group protein B1 | | | |
| A0A0J9YUD8 | | | | |
| D3YVC6 | | | | |
| D3YZ18 | | | | |
| G3X8T9 | Serine protease | 1.50 ± 0.76 | 0.78 ± 0.44 | 48.15 |
| Q91WP6 | inhibitor A3N | | | |
| H7BWY0 | | | | |
| Q6P4P1 | | | | |
| A0A0R4IZY6 | Myeloblastin | 1.50 ± 1.41 | 1.00 ± 1.33 | 33.33 |
| Q61096 | | | | |
| F6ZK01 | | | | |
| E9PZ00 | Prosaposin | 1.50 ± 1.07 | 0.38 ± 0.74 | 75.00 |
| Q8BFQ1 | | | | |
| K3W4L3 | | | | |
| J3QPG5 | | | | |
| Q61207 | | | | |
| P68254 | 14-3-3 protein | 1.50 ± 1.07 | 1.78 ± 0.83 | −18.52 |
| | theta | | | |
| Q839G8 | | 1.43 ± 1.13 | 0.40 ± 0.84 | 72.00 |
| Q9CPX4 | Ferritin | 1.43 ± 1.40 | 0.00 ± 0.00 | 100.00 |
| A0A1B0GR60 | | | | |
| P29391 | | | | |
| A0A1B0GRH4 | | | | |
| P49945 | | | | |
| A0A1Y7VNT9 | | | | |
| Q9WVA4 | Transgelin-2 | 1.38 ± 2.00 | 0.44 ± 0.73 | 67.68 |
| A0A0A6YXG6 | | | | |
| Q9R1Q8 | | | | |
| Q61599 | Rho GDP- | 1.38 ± 1.77 | 0.40 ± 0.52 | 70.91 |
| D3YWL7 | dissociation | | | |
| A0A0N4SVH4 | inhibitor 2 | | | |
| Q8VEK3 | Heterogeneous | 1.38 ± 2.00 | 0.50 ± 0.71 | 63.64 |
| | nuclear | | | |
| | ribonucleoprotein | | | |
| | U | | | |
| P09103 | Protein disulfide- | 1.38 ± 2.00 | 1.00 ± 1.05 | 27.27 |
| E9Q8G8 | isomerase | | | |
| Q9JM76 | Actin-related | 1.38 ± 1.60 | 0.30 ± 0.67 | 78.18 |
| H7BWZ3 | protein 2/3 | | | |
| A0A0G2JFK7 | complex subunit | | | |
| D3Z2F7 | 3 | | | |
| D3Z2F8 | | | | |
| P29351 | Tyrosine-protein | 1.38 ± 1.85 | 0.11 ± 0.33 | 91.92 |
| | phosphatase non- | | | |
| | receptor type 6 | | | |
| P41245 | Matrix | 1.38 ± 1.60 | 0.30 ± 0.67 | 78.18 |
| | metalloproteinase- | | | |
| | 9 | | | |
| Q91Z25 | Actin-related | 1.38 ± 1.85 | 0.11 ± 0.33 | 91.92 |
| Q9WV32 | protein 2/3 | | | |
| F6VVE6 | complex subunit | | | |
| D3Z6S0 | 1B | | | |
| F6THG2 | | | | |
| G3UY29 | Ras-related | 1.38 ± 1.60 | 0.78 ± 0.83 | 43.43 |
| E9Q3P9 | protein Rab-11A | | | |
| F8WGS1 | Ras-related | | | |
| P62492 | protein Rab-11B | | | |
| P46638 | | | | |
| G3UZD3 | | | | |
| G3UZL4 | | | | |
| D3YYB3 | | | | |
| Q9QZQ8 | Core histone | 1.38 ± 1.60 | 0.56 ± 0.53 | 59.60 |
| Q8CCK0 | macro-H2A.1 | | | |
| A6BLY7 | Keratin, type I | 1.38 ± 0.52 | 1.33 ± 0.50 | 3.03 |
| | cytoskeletal 28 | | | |
| Q60590 | Alpha-1-acid | 1.38 ± 1.06 | 0.00 ± 0.00 | 100.00 |
| | glycoprotein 1 | | | |
| Q3U9G9 | Lamin-B receptor | 1.38 ± 1.77 | 0.22 ± 0.67 | 83.84 |
| A0A0A6YWS3 | | | | |
| A0A0A6YXW3 | | | | |
| A0A0A6YXT6 | | | | |
| A0A0A6YW01 | | | | |
| A0A0A6YY12 | | | | |
| P62827 | GTP-binding | 1.38 ± 0.74 | 0.50 ± 0.53 | 63.64 |
| Q14AA6 | nuclear protein | | | |
| Q61820 | Ran | | | |
| P10639 | Thioredoxin | 1.38 ± 0.74 | 0.60 ± 0.52 | 56.36 |
| Q8VCU2 | Phosphatidylinosi | 1.38 ± 1.06 | 0.00 ± 0.00 | 100.00 |
| O70362 | tol-glycan- | | | |
| | specific | | | |
| | phospholipase D | | | |
| A0A0A6YY34 | Glutathione | 1.38 ± 1.51 | 0.00 ± 0.00 | 100.00 |
| A0A0A6YVV2 | peroxidase | | | |
| P11352 | Glutathione | | | |
| | peroxidase 1 | | | |
| P01897 | H-2 class I | 1.38 ± 1.51 | 0.00 ± 0.00 | 100.00 |
| P01899 | histocompatibility | | | |
| P01896 | antigen, L-D | | | |
| | alpha chain | | | |
| Q91Z98 | Chitinase-like | 1.38 ± 1.51 | 0.50 ± 0.71 | 63.64 |
| | protein 4 | | | |
| P14211 | Calreticulin | 1.33 ± 1.21 | 0.30 ± 0.67 | 77.50 |
| G3X977 | Inter-alpha- | | | |
| Q61703 | trypsin inhibitor | | | |
| Q3UEG7 | heavy chain H2 | | | |
| CON_Q9TRI1 | | 1.33 ± 0.52 | 0.11 ± 0.33 | 91.67 |
| Q61171 | Peroxiredoxin-2 | 1.29 ± 0.49 | 1.50 ± 1.08 | −16.67 |
| D3Z4A4 | | | | |
| P14152 | Malate | 1.29 ± 1.11 | 0.67 ± 0.71 | 48.15 |
| | dehydrogenase, | | | |
| | cytoplasmic | | | |
| Q06770 | Corticosteroid- | 1.29 ± 1.11 | 0.00 ± 0.00 | 100.00 |
| | binding globulin | | | |
| P28293 | Cathepsin G | 1.29 ± 1.11 | 0.11 ± 0.33 | 91.36 |
| Q3THE2 | Myosin | 1.29 ± 0.49 | 0.60 ± 0.52 | 53.33 |
| Q6ZWQ9 | regulatory light | | | |
| Q9CQ19 | chain 12B | | | |
| | Myosin | | | |
| | regulatory light | | | |
| | polypeptide 9 | | | |
| Q8CI94 | Glycogen | 1.25 ± 1.83 | 1.80 ± 1.87 | −44.00 |
| | phosphorylase, | | | |
| | brain form | | | |
| P50516 | V-type proton | 1.25 ± 1.75 | 0.00 ± 0.00 | 100.00 |
| D3Z1B9 | ATPase catalytic | | | |
| D3YWH3 | subunit A | | | |
| D3YZ23 | | | | |
| Q61093 | Cytochrome b- | 1.25 ± 1.75 | 0.11 ± 0.33 | 91.11 |
| E9Q7S3 | 245 heavy chain | | | |
| E9Q802 | | | | |
| P49710 | Hematopoietic | 1.25 ± 1.75 | 0.00 ± 0.00 | 100.00 |
| E9Q4E5 | lineage cell- | | | |
| | specific protein | | | |
| Q6P069 | Sorcin | 1.25 ± 1.39 | 0.30 ± 0.48 | 76.00 |
| A0A1L1SQA8 | 40S ribosomal | 1.25 ± 0.46 | 0.89 ± 0.60 | 28.89 |
| P62852 | protein S25 | | | |
| Q9QXC1 | Fetuin-B | 1.25 ± 0.46 | 0.11 ± 0.33 | 91.11 |
| Q6YJU1 | | | | |
| P68510 | 14-3-3 protein eta | 1.25 ± 0.89 | 1.50 ± 1.18 | −20.00 |
| A2AE91 | | 1.25 ± 0.46 | 1.30 ± 0.48 | −4.00 |
| Q8R516 | | | | |
| Q80X90 | Filamin-B | 1.17 ± 1.17 | 4.00 ± 4.76 | −242.86 |
| P51885 | Lumican | 1.17 ± 0.41 | 0.00 ± 0.00 | 100.00 |
| CON_Q05443 | | | | |
| Q9CZM2 | 60S ribosomal | 1.17 ± 0.41 | 0.56 ± 0.53 | 52.38 |
| B8JKK2 | protein L15 | | | |
| | Ribosomal | | | |
| | protein L15 | | | |
| P21550 | Beta-enolase | 1.17 ± 0.41 | 0.00 ± 0.00 | 100.00 |
| G3X9D9 | Involucrin | 1.14 ± 0.90 | 2.90 ± 2.42 | −153.75 |
| P48997 | | | | |
| Q3U3V1 | Coagulation | 1.14 ± 0.90 | 0.00 ± 0.00 | 100.00 |
| O88947 | factor X | | | |
| A0A1W2P6F6 | Myosin light | 1.14 ± 0.90 | 0.11 ± 0.33 | 90.28 |
| A0A1W2P7Q9 | polypeptide 6 | | | |
| Q60605 | | | | |
| A0A1W2P6G5 | | | | |
| P60766 | Cell division | 1.14 ± 0.38 | 0.67 ± 0.71 | 41.67 |
| | control protein 42 | | | |
| | homolog | | | |
| P11680 | Properdin | 1.14 ± 0.38 | 0.20 ± 0.42 | 82.50 |
| H7BWY6 | Retinol-binding | 1.14 ± 0.38 | 0.11 ± 0.33 | 90.28 |
| Q00724 | protein 4 | | | |
| A0A494B9P3 | | | | |
| A0A494B9T2 | | | | |
| Q8R2S8 | CD177 antigen | 1.13 ± 1.36 | 0.70 ± 1.49 | 37.78 |
| Q3SXK0 | Uroplakin-2 | 1.13 ± 0.83 | 1.22 ± 1.20 | −8.64 |
| P38575 | | | | |
| P03953 | Complement | 1.13 ± 1.36 | 0.30 ± 0.67 | 73.33 |
| | factor D | | | |
| Q02053 | Ubiquitin-like | 1.13 ± 1.55 | 0.56 ± 0.53 | 50.62 |
| P31254 | modifier- | | | |
| | activating | | | |
| | enzyme 1 | | | |
| Q99LB4 | Macrophage- | 1.13 ± 1.55 | 0.67 ± 0.71 | 40.74 |
| P24452 | capping protein | | | |
| D3YZN3 | | | | |
| D3YU77 | | | | |
| D3YTL5 | | | | |
| D3Z4K5 | | | | |
| S4R293 | Neutrophil | 1.13 ± 1.36 | 0.11 ± 0.33 | 90.12 |
| F8WH69 | cytosol factor 1 | | | |
| Q09014 | | | | |
| P06684 | Complement C5 | 1.13 ± 1.25 | 0.00 ± 0.00 | 100.00 |
| P14131 | 40S ribosomal | 1.13 ± 1.46 | 0.33 ± 0.50 | 70.37 |
| | protein S16 | | | |
| P26262 | Plasma kallikrein | 1.13 ± 0.99 | 0.00 ± 0.00 | 100.00 |
| P62918 | 60S ribosomal | 1.13 ± 0.83 | 1.00 ± 0.94 | 11.11 |
| | protein L8 | | | |
| E9Q499 | Serine protease | 1.13 ± 0.35 | 0.78 ± 0.44 | 30.86 |
| F2Z405 | inhibitor A3G | | | |
| Q512A0 | | | | |
| Q9R0P9 | Ubiquitin | 1.00 ± 1.00 | 1.80 ± 1.32 | −80.00 |
| | carboxyl-terminal | | | |
| | hydrolase | | | |
| | isozyme L1 | | | |
| O70475 | UDP-glucose 6- | 1.00 ± 0.76 | 1.90 ± 0.99 | −90.00 |
| D3YXP9 | dehydrogenase | | | |
| A0A0A6YW77 | Neutrophil | 1.00 ± 1.20 | 0.11 ± 0.33 | 88.89 |
| P11672 | gelatinase- | | | |
| | associated | | | |
| | lipocalin | | | |
| Q61792 | LIM and SH3 | 1.00 ± 1.20 | 0.33 ± 0.50 | 66.67 |
| A2A6G9 | domain protein 1 | | | |
| A2A6H0 | | | | |
| A2A6G7 | | | | |
| A2A6G8 | | | | |
| A2A6G0 | | | | |
| A2A6G6 | | | | |
| P70460 | Vasodilator- | 1.00 ± 1.41 | 0.30 ± 0.67 | 70.00 |
| | stimulated | | | |
| | phosphoprotein | | | |
| D3Z1T4 | Guanine | 1.00 ± 0.89 | 0.00 ± 0.00 | 100.00 |
| D3Z1M1 | nucleotide- | | | |
| D3YZX3 | binding protein | | | |
| E9QKR0 | G(I)/G(S)/G(T) | | | |
| P62880 | subunit beta-2 | | | |
| H3BLF7 | | | | |
| H3BKR2 | | | | |
| P29387 | | | | |
| P62874 | | | | |
| E9PWM7 | | | | |
| Q61011 | | | | |
| P84084 | ADP-ribosylation | 1.00 ± 1.31 | 0.60 ± 0.84 | 40.00 |
| | factor 5 | | | |
| Q5RKN9 | F-actin-capping | 1.00 ± 1.31 | 0.00 ± 0.00 | 100.00 |
| P47753 | protein subunit | | | |
| A0A0G2JE27 | alpha-1 | | | |
| P61358 | 60S ribosomal | 1.00 ± 0.76 | 0.50 ± 0.71 | 50.00 |
| A2A4Q0 | protein L27 | | | |
| A0A0R4J0R1 | Vesicle- | 1.00 ± 1.20 | 0.00 ± 0.00 | 100.00 |
| O70404 | associated | | | |
| A0A0U1RPE8 | membrane | | | |
| | protein 8 | | | |
| P01837 | Ig kappa chain C | 1.00 ± 0.00 | 0.00 ± 0.00 | 100.00 |
| | region | | | |
| P01887 | Beta-2- | 1.00 ± 0.76 | 1.00 ± 0.00 | 0.00 |
| | microglobulin | | | |
| P30115 | Glutathione S- | 1.00 ± 0.00 | 1.50 ± 0.71 | −50.00 |
| Q6P8Q0 | transferase A3 | | | |
| P13745 | | | | |
| A0A087WQI6 | | | | |
| D3Z6A6 | | | | |
| D3YZV3 | | | | |
| P10648 | | | | |
| P60335 | Poly(rC)-binding | 1.00 ± 1.07 | 0.80 ± 0.79 | 20.00 |
| | protein 1 | | | |
| O08997 | Copper transport | 1.00 ± 0.00 | 0.00 ± 0.00 | 100.00 |
| | protein ATOX1 | | | |
| A0A0R4J043 | Meprin A subunit | 1.00 ± 0.76 | 0.78 ± 0.44 | 22.22 |
| P28825 | alpha | | | |
| I7HPY0 | SH3 domain- | 1.00 ± 0.00 | 1.00 ± 0.00 | 0.00 |
| Q91VW3 | binding glutamic | | | |
| | acid-rich-like | | | |
| | protein 3 | | | |
| Q3UA72 | Actin-related | 1.00 ± 0.00 | 0.60 ± 0.52 | 40.00 |
| Q9CPW4 | protein 2/3 | | | |
| | complex subunit 5 | | | |
| P61027 | Ras-related | 1.00 ± 0.00 | 0.89 ± 0.93 | 11.11 |
| | protein Rab-10 | | | |
| P57096 | Prostate stem cell | 1.00 ± 0.00 | 0.33 ± 0.50 | 66.67 |
| | antigen | | | |
| P07361 | Alpha-1-acid | 1.00 ± 0.82 | 0.00 ± 0.00 | 100.00 |
| | glycoprotein 2 | | | |
| O09043 | Napsin-A | 1.00 ± 0.00 | 0.80 ± 0.42 | 20.00 |
| P62242 | 40S ribosomal | 0.88 ± 1.36 | 0.78 ± 0.83 | 11.11 |
| | protein S8 | | | |
| P59999 | Actin-related | 0.88 ± 0.99 | 0.33 ± 0.50 | 61.90 |
| Q3TX55 | protein 2/3 | | | |
| E9PWA7 | complex subunit 4 | | | |
| G3UX26 | Voltage- | 0.88 ± 0.99 | 0.25 ± 0.46 | 71.43 |
| Q60930 | dependent anion- | | | |
| D3YZT5 | selective channel | | | |
| A0A286YCR8 | protein 2 | | | |
| D3YUN8 | | | | |
| Q9Z183 | Protein-arginine | 0.88 ± 1.13 | 0.00 ± 0.00 | 100.00 |
| E9QAM4 | deiminase type-4 | | | |
| Q9Z184 | | | | |
| Q9CR57 | 60S ribosomal | 0.88 ± 0.64 | 0.67 ± 0.71 | 23.81 |
| | protein L14 | | | |
| P19783 | Cytochrome c | 0.88 ± 0.99 | 0.30 ± 0.67 | 65.71 |
| | oxidase subunit 4 | | | |
| | isoform 1, | | | |
| | mitochondrial | | | |
| Q62093 | Serine/arginine- | 0.88 ± 0.99 | 0.40 ± 0.52 | 54.29 |
| | rich splicing | | | |
| | factor 2 | | | |
| P84078 | ADP-ribosylation | 0.88 ± 1.13 | 0.40 ± 0.52 | 54.29 |
| P61205 | factor 1 | | | |
| Q8BSL7 | | | | |
| D3YV25 | | | | |
| E9Q2C2 | | | | |
| A2A6T9 | | | | |
| E9PZW0 | Desmoplakin | 0.86 ± 0.69 | 1.29 ± 1.25 | −50.00 |
| E9Q557 | | | | |
| B1ARA3 | 60S ribosomal | 0.86 ± 1.21 | 0.67 ± 0.71 | 22.22 |
| P61255 | protein L26 | | | |
| B1ARA5 | | | | |
| Q835M3 | | 0.86 ± 0.69 | 0.11 ± 0.33 | 87.04 |
| Q8C266 | Ras-related | 0.86 ± 0.69 | 0.33 ± 0.50 | 61.11 |
| Q9CQD1 | protein Rab-5A | | | |
| P61021 | | | | |
| P35278 | | | | |
| A2A5F6 | | | | |
| A2A5F5 | | | | |
| Q91V55 | 40S ribosomal | 0.83 ± 0.75 | 0.60 ± 0.84 | 28.00 |
| D3YYM6 | protein S5 | | | |
| D3Z1S8 | | | | |
| P97461 | | | | |
| H3BK03 | Serum | 0.83 ± 0.75 | 0.00 ± 0.00 | 100.00 |
| P52430 | paraoxonase/aryl | | | |
| H3BLB8 | esterase 1 | | | |
| A0A075B5P5 | Ig gamma-3 | 0.83 ± 0.75 | 0.00 ± 0.00 | 100.00 |
| A0A1Y7VJN6 | chain C region | | | |
| P03987 | | | | |
| Q61878 | Bone marrow | 0.75 ± 0.89 | 0.00 ± 0.00 | 100.00 |
| | proteoglycan | | | |
| Q9D8N0 | Elongation factor | 0.75 ± 0.71 | 1.00 ± 1.22 | −33.33 |
| | 1-gamma | | | |
| P00329 | Alcohol | 0.75 ± 0.46 | 2.00 ± 1.94 | −166.67 |
| | dehydrogenase 1 | | | |
| A0A494BBB0 | Calpain-1 | 0.75 ± 1.39 | 1.00 ± 1.12 | −33.33 |
| O35350 | catalytic subunit | | | |
| A0A1L1SU53 | Twinfilin-2 | 0.75 ± 0.89 | 0.00 ± 0.00 | 100.00 |
| Q9Z0P5 | | | | |
| A0A087WRG4 | | | | |
| A0A1L1STC8 | | | | |
| Q91XL1 | | 0.75 ± 0.89 | 0.00 ± 0.00 | 100.00 |
| F7CAZ6 | F-actin-capping | 0.75 ± 0.89 | 0.00 ± 0.00 | 100.00 |
| A2AMW0 | protein subunit | | | |
| P47757 | beta | | | |
| A0A0A0MQI9 | | | | |
| F6YHZ8 | | | | |
| Q8CB58 | Polypyrimidine | 0.75 ± 0.89 | 0.11 ± 0.33 | 85.19 |
| Q8BGJ5 | tract-binding | | | |
| Q922I7 | protein 3 | | | |
| F7C521 | | | | |
| F7DCW4 | | | | |
| E9QMW9 | | | | |
| G8JL74 | | | | |
| G3UXA6 | | | | |
| Q8BHD7 | | | | |
| P17225 | | | | |
| A0A075B5P4 | Ig gamma-1 | 0.75 ± 0.46 | 0.33 ± 0.50 | 55.56 |
| A0A0A6YWR2 | chain C region | | | |
| P01868 | secreted form | | | |
| P01869 | | | | |
| G3UYV7 | 40S ribosomal | 0.75 ± 0.46 | 0.56 ± 0.53 | 25.93 |
| P62858 | protein S28 | | | |
| E9PUM5 | | 0.75 ± 0.89 | 0.20 ± 0.42 | 73.33 |
| E9Q8B6 | | | | |
| E9Q8B5 | | | | |
| Q61405 | | | | |
| Q3UDR8 | Protein YIPF3 | 0.75 ± 0.46 | 0.75 ± 0.46 | 0.00 |
| | Protein YIPF3, | | | |
| | N-terminally | | | |
| | processed | | | |
| P50396 | Rab GDP | 0.75 ± 0.89 | 0.88 ± 1.13 | −16.67 |
| D6RI86 | dissociation | | | |
| | inhibitor alpha | | | |
| P45377 | Aldose reductase- | 0.71 ± 0.95 | 1.50 ± 1.35 | −110.00 |
| P21300 | related protein 2 | | | |
| | Aldose reductase- | | | |
| | related protein 1 | | | |
| P97351 | 40S ribosomal | 0.71 ± 0.95 | 0.90 ± 0.88 | −26.00 |
| | protein S3a | | | |
| P62264 | 40S ribosomal | 0.71 ± 0.95 | 1.25 ± 0.89 | −75.00 |
| D3YVF4 | protein S14 | | | |
| D3Z711 | | | | |
| M0QWU8 | Acyl-CoA- | 0.71 ± 0.49 | 0.20 ± 0.42 | 72.00 |
| P31786 | binding protein | | | |
| P49182 | Heparin cofactor 2 | 0.71 ± 1.11 | 0.00 ± 0.00 | 100.00 |
| B1B1A8 | Myosin light | 0.71 ± 0.49 | 0.11 ± 0.33 | 84.44 |
| Q6PDN3 | chain kinase | | | |
| P97315 | Cysteine and | 0.71 ± 0.49 | 0.00 ± 0.00 | 100.00 |
| | glycine-rich | | | |
| | protein 1 | | | |
| P51437 | Cathelin-related | 0.71 ± 0.49 | 0.40 ± 0.52 | 44.00 |
| | antimicrobial | | | |
| | peptide | | | |
| P07515 | | 0.71 ± 0.49 | 0.11 ± 0.33 | 84.44 |
| E9PUZ8 | C4b-binding | 0.71 ± 0.49 | 0.00 ± 0.00 | 100.00 |
| P08607 | protein | | | |
| Q830Q8 | | 0.71 ± 0.49 | 0.40 ± 0.52 | 44.00 |
| P06801 | NADP-dependent | 0.67 ± 0.52 | 1.63 ± 1.69 | −143.75 |
| Q3TQP6 | malic enzyme | | | |
| | Malic enzyme | | | |
| P05366 | Serum amyloid | 0.67 ± 0.52 | 0.20 ± 0.42 | 70.00 |
| A0A1B0GQV5 | A-1 protein | | | |
| P05367 | Serum amyloid | | | |
| | A-2 protein | | | |
| | Amyloid protein A | | | |
| D3Z1V4 | Phosphatidyletha- | 0.67 ± 0.52 | 0.33 ± 0.50 | 50.00 |
| P70296 | nolamine-binding | | | |
| | protein 1 | | | |
| Q76MZ3 | Serine/threonine- | 0.63 ± 1.19 | 1.00 ± 1.12 | −60.00 |
| G3UWL2 | protein | | | |
| | phosphatase 2A | | | |
| | 65 kDa | | | |
| | regulatory | | | |
| | subunit A alpha | | | |
| | isoform | | | |
| P26350 | Prothymosin | 0.63 ± 0.74 | 0.44 ± 0.73 | 28.89 |
| A0A087WP98 | alpha | | | |
| A0A087WPN6 | Prothymosin | | | |
| A0A087WQN2 | alpha, N- | | | |
| | terminally | | | |
| | processed | | | |
| | Thymosin alpha | | | |
| A0A2C9F2D2 | Annexin A7 | 0.63 ± 1.19 | 0.80 ± 0.92 | −28.00 |
| Q07076 | | | | |
| A0A286YCW4 | | | | |
| F6UFG6 | Acidic leucine- | 0.63 ± 0.74 | 0.25 ± 0.46 | 60.00 |
| D3YYE1 | rich nuclear | | | |
| D3Z7M9 | phosphoprotein | | | |
| O35381 | 32 family | | | |
| Q9EST5 | member A | | | |
| Q64G17 | | | | |
| P26443 | Glutamate | 0.63 ± 0.74 | 0.70 ± 0.67 | −12.00 |
| | dehydrogenase 1, | | | |
| | mitochondrial | | | |
| Q921R2 | 40S ribosomal | 0.63 ± 0.74 | 0.40 ± 0.52 | 36.00 |
| P62301 | protein S13 | | | |
| A0A0UIRQ71 | | | | |
| P97425 | Eosinophil | 0.63 ± 0.74 | 0.00 ± 0.00 | 100.00 |
| | cationic protein 2 | | | |
| P27005 | Protein S100-A8 | 0.63 ± 0.74 | 0.11 ± 0.33 | 82.22 |
| Q839F8 | | 0.63 ± 0.74 | 0.00 ± 0.00 | 100.00 |
| A0A494BA97 | Osteoclast- | 0.63 ± 0.74 | 0.00 ± 0.00 | 100.00 |
| Q62422 | stimulating factor 1 | | | |
| A0A494B943 | | | | |
| Q6ZWV7 | 60S ribosomal | 0.60 ± 0.55 | 0.56 ± 0.53 | 7.41 |
| | protein L35 | | | |
| P80314 | T-complex | 0.57 ± 1.51 | 1.11 ± 1.27 | −94.44 |
| A0A1W2P7B7 | protein 1 subunit | | | |
| A0A1W2P828 | beta | | | |
| A0A1W2P871 | | | | |
| A0A1W2P6Q3 | | | | |
| Q99PT1 | Rho GDP- | 0.50 ± 0.53 | 0.44 ± 0.73 | 11.11 |
| | dissociation | | | |
| | inhibitor 1 | | | |
| D3YVG1 | Septin-1 | 0.50 ± 0.53 | 0.00 ± 0.00 | 100.00 |
| D3Z3V3 | | | | |
| P42209 | | | | |
| O08553 | Dihydropyrimidinase- | 0.50 ± 0.53 | 0.00 ± 0.00 | 100.00 |
| | related | | | |
| | protein 2 | | | |
| Q9D6Y7 | Mitochondrial | 0.50 ± 0.53 | 0.11 ± 0.33 | 77.78 |
| A0A1B0GT40 | peptide | | | |
| | methionine | | | |
| | sulfoxide | | | |
| | reductase | | | |
| Q9WTL2 | Ras-related | 0.50 ± 0.53 | 0.60 ± 0.84 | −20.00 |
| | protein Rab-25 | | | |
| Q9DCM0 | Persulfide | 0.50 ± 0.53 | 0.00 ± 0.00 | 100.00 |
| | dioxygenase | | | |
| | ETHE1, | | | |
| | mitochondrial | | | |
| P97426 | Eosinophil | 0.50 ± 0.53 | 0.00 ± 0.00 | 100.00 |
| O35290 | cationic protein 1 | | | |
| | Eosinophil | | | |
| | cationic-type | | | |
| | ribonuclease 3 | | | |
| P84104 | Serine/arginine- | 0.50 ± 0.53 | 0.00 ± 0.00 | 100.00 |
| | rich splicing | | | |
| | factor 3 | | | |
| Q60932 | Voltage- | 0.50 ± 0.53 | 0.00 ± 0.00 | 100.00 |
| | dependent anion- | | | |
| | selective channel | | | |
| | protein 1 | | | |
| Q9QXT9 | Zinc finger | 0.50 ± 0.53 | 0.00 ± 0.00 | 100.00 |
| | protein 354B | | | |
| P80316 | T-complex | 0.43 ± 0.53 | 0.88 ± 0.64 | −104.17 |
| E0CZA1 | protein 1 subunit | | | |
| | epsilon | | | |
| P63038 | 60 kDa heat | 0.43 ± 0.53 | 1.00 ± 0.94 | −133.33 |
| | shock protein, | | | |
| | mitochondrial | | | |
| D3Z4N2 | Osteopontin | 0.43 ± 0.53 | 0.71 ± 0.49 | −66.67 |
| F8WIP8 | | | | |
| P10923 | | | | |
| P97429 | Annexin A4 | 0.38 ± 0.74 | 1.11 ± 1.17 | −196.30 |
| F7ANV6 | Annexin | | | |
| D3Z0S1 | | | | |
| A0A0N4SW89 | | | | |
| A0A0N4SV57 | | | | |
| S4R1F2 | | | | |
| P25444 | 40S ribosomal | 0.38 ± 0.74 | 0.11 ± 0.33 | 70.37 |
| D3YVC1 | protein S2 | | | |
| D3YWJ3 | | | | |
| P60867 | 40S ribosomal | 0.33 ± 0.52 | 0.78 ± 0.83 | −133.33 |
| | protein S20 | | | |
| A0A0R4J1C2 | Calpain small | 0.33 ± 0.52 | 0.60 ± 0.52 | −80.00 |
| A0A0R4IZW8 | subunit 1 | | | |
| O88456 | Calpain small | | | |
| Q9D7J7 | subunit 2 | | | |
| Q9CX34 | Suppressor of G2 | 0.29 ± 0.76 | 0.56 ± 0.53 | −94.44 |
| | allele of SKP1 | | | |
| | homolog | | | |
| P14115 | 60S ribosomal | 0.25 ± 0.46 | 0.60 ± 0.52 | −140.00 |
| | protein L27a | | | |
| G3UYI4 | 26S proteasome | 0.14 ± 0.38 | 0.56 ± 0.53 | −288.89 |
| G3UYL3 | non-ATPase | | | |
| Q8BG32 | regulatory | | | |
| | subunit 11 | | | |
| O55234 | Proteasome | 0.00 ± 0.00 | 0.80 ± 0.79 | N/A |
| Q8BTY5 | subunit beta type-5 | | | |
| Q921L6 | Src substrate | 0.00 ± 0.00 | 1.00 ± 1.22 | N/A |
| Q60598 | cortactin | | | |

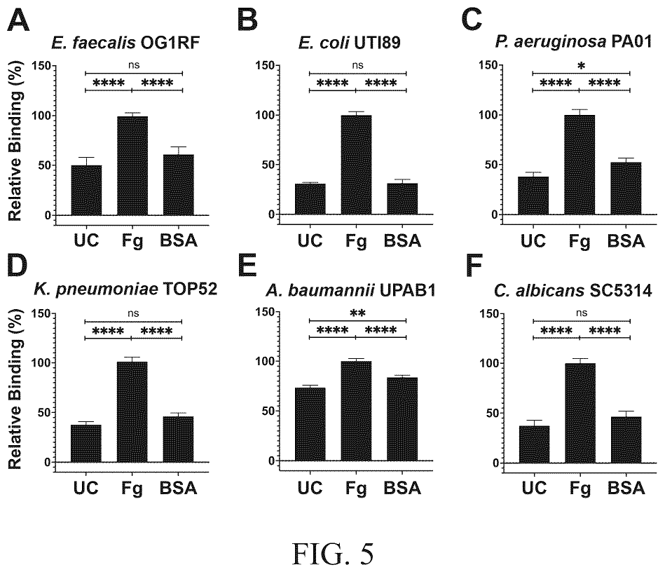

Uropathogen binding to Fg- (Fibrinogen), BSA- (bovine serum albumin), and uncoated silicone (UC) were compared as shown in FIG. 5, panels A-F. The standard error of the mean (SEM) are represented as error bars. Between 3-5 replicates of n=4-12 each were performed for each pathogen and condition. Differences between groups were tested for significance using the Mann-Whitney U test. *P≤0.05, **P≤0.01, ***P≤0.001; ****P≤ns, difference not significant.

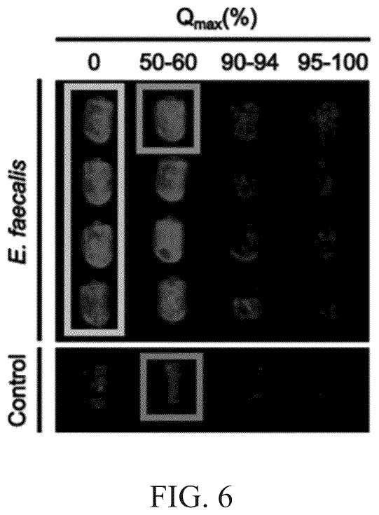

FIG. 6 shows how the relative binding percentage was calculated for FIG. 5, panels A-F. Relative binding percentage was calculated as the sample (e.g., the signal in the blue square) minus the background (e.g., the signal in the purple square) divided by the average of the 0% Qₘₐₓ samples. The 0% Qₘₐₓ samples are identified in the green square. A visual description of this calculation is included in the email with this document.

Fg significantly enhanced the binding to the catheter for all uropathogens when compared with uncoated and BSA-coated silicone catheters (FIG. 5, panels A-F). Interestingly, P. aeruginosa and A. baumannii binding to BSA-coated silicone was ˜14% and ˜10% higher than uncoated controls, respectively (FIG. 5, panels C and E). The results indicate a role for other host-secreted proteins during infection. However, these values were still significantly lower than the increase in binding observed on Fg-coated silicone (FIG. 5, panels C and E). Taken together, these data suggest that uropathogen interaction with host-proteins deposited on silicone surfaces, particularly Fg, increases the ability of uropathogens to colonize urinary catheters.

### Characterization of Liquid-Infused Catheters to Prevent Host-Protein Deposition.

Based on the exploitative interaction of uropathogens with deposited Fg, a material to prevent protein deposition would also reduce microbial colonization. Liquid-infused surfaces resist protein and bacterial fouling. Inventors developed a LIS material by modifying medical-grade silicone using inert trimethyl-terminated polydimethylsiloxane fluid (referred to as “silicone oil” in the present example).

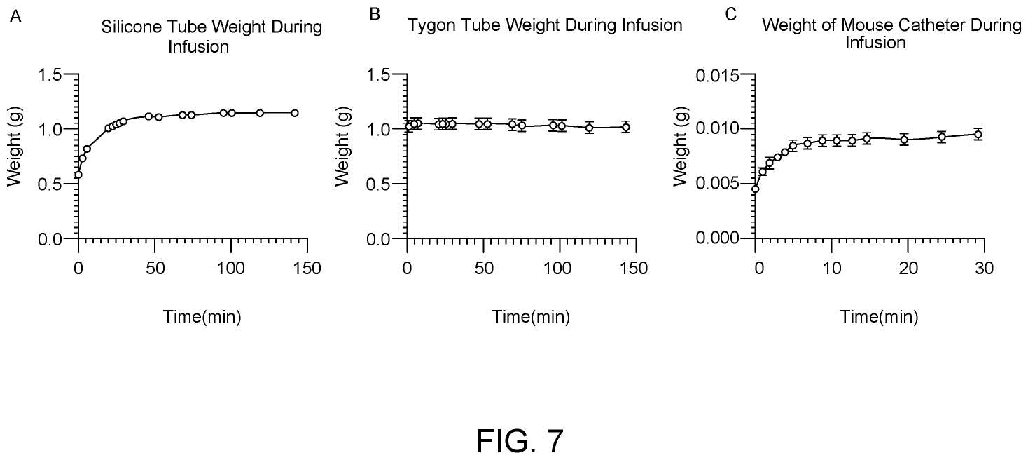

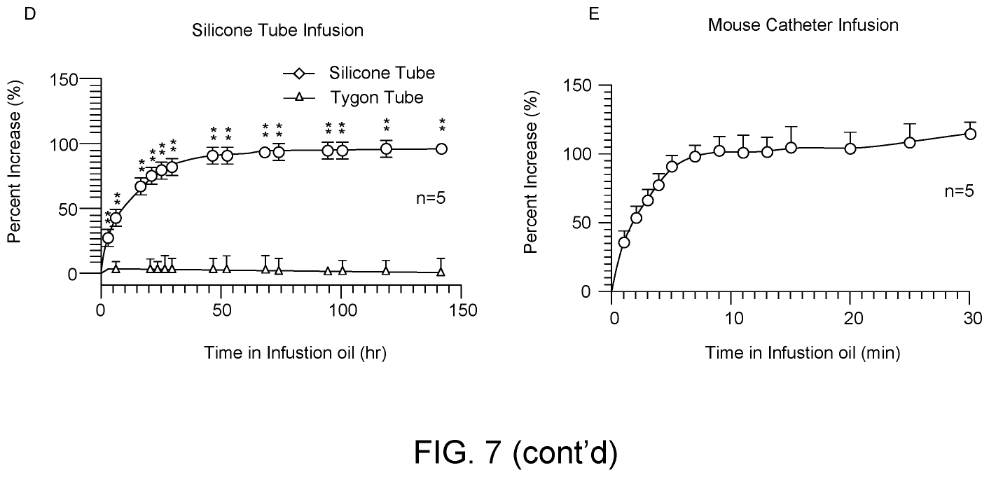

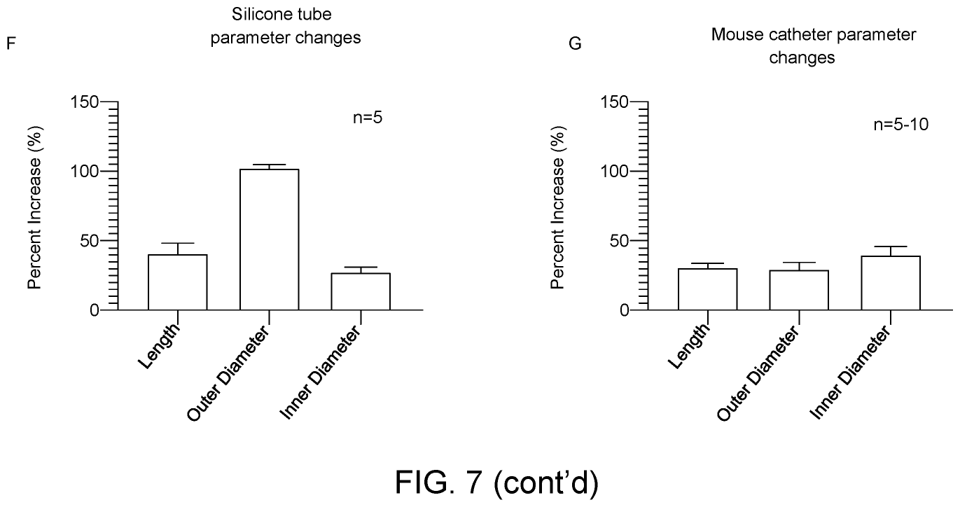

FIG. 7 shows a series of panels characterizing silicone oil infusion of silicone tubing, Tygon® tubing, and a mouse catheter. In FIG. 7, panel A, the weight of silicone tubes were measured at designated time points before and during silicone oil infusion. The mean (±SEM) of n=5 silicone tubes over infusion time is shown in this figure. In FIG. 7, panel B, the weight of Tygon® tubes were measured in designated time points before and during silicone oil infusion, the mean (±SEM) of n=5 silicone tubes over infusion time is shown in this figure. In FIG. 7, panel C, the weight of mouse catheters were measured at designated time points during and before (i.e., at t=0) silicone oil infusion. The mean (±SEM) of n=5 mouse catheter over infusion time is shown. In FIG. 7, panel D, kinetics of silicone oil infusion is shown for silicone and Tygon® tubes. In FIG. 7, panel E, kinetics of silicone oil infusion is shown for mouse silicone catheters. The change in length, outer diameter, and inner diameter of silicone catheters (n=5) is shown in FIG. 7, panel F. The change in length, outer diameter, and inner diameter of mouse catheters (n=5-10) is shown in FIG. 7, panel G. were measured before and after infusion and the percentage change was calculated.

Analysis of the silicone oil's infusion rate into silicone tubing showed a significant increase in silicone weight during the first 3 days of infusion then a gradual decrease in infusion until a plateau was reached after about 50 hrs. The change in raw weight is shown in FIG. 7, panel A. The percent change in weight of the tubing is shown in FIG. 7, panel D in black (upper line). Plastic Tygon® tubes (non-silicone) were used as negative controls. FIG. 7, panel D in grey (lower line) shows the percent change in weight of the Tygon® tubes. FIG. 7, panel B shows the weight of the Tygon® tubing during the infusion. As can be seen from these panels, Tygon® is not suitable for infusion with silicone oil by immersion.

Full infusion of mouse silicone catheters was achieved by 10 min as shown in FIG. 7, panel C and FIG. 7, panel E. Investigation of silicone tube dimensions showed an increase in length, outer and inner diameter of ˜41.3%, ˜103.1%, and ˜27.6%, respectively (FIG. 7, panel F) and mouse catheters showed an increase of ˜30.7%, ˜28.7%, and ˜39.8%, respectively (FIG. 7, panel G).

### LIS Modification Reduces Fg Deposition and Microbial Binding In Vitro.

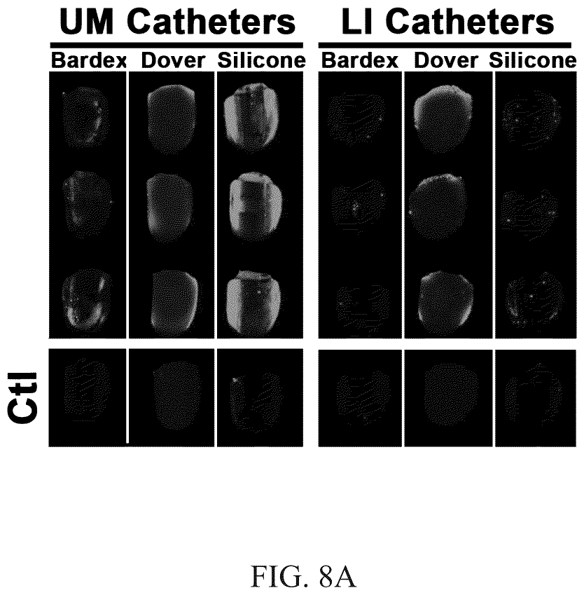

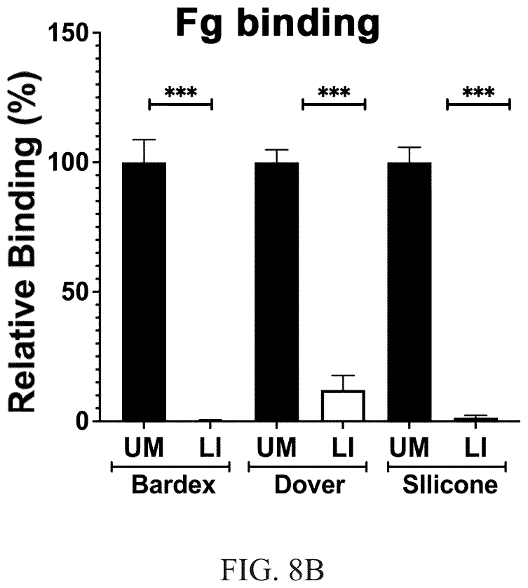

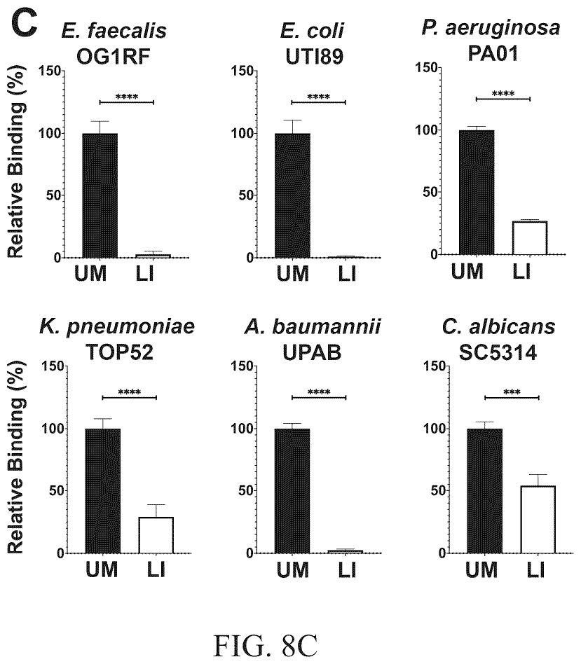

The ability of the LIS-catheters to reduce Fg deposition in vitro was tested for a silicone-oil impregnated, medical-grade silicone material and two commercially available urinary catheters, Dover and Bardex. Unmodified (UM) versions of each material were used as controls. Each was incubated with Fg overnight and assessed by IF. Fg deposition was reduced in all LIS-catheters, showing ˜90% decrease on the Dover catheter and ˜100% on the Bardex catheter and medical-grade silicone tubings when compared with the corresponding unmodified controls (FIG. 8A and FIG. 8B). FIG. 8A shows the absorption of fibrinogen (green) to Bardex, Dover, and silicone catheters which are unmodified (UM Catheters) as compared to silicone oil impregnated, “liquid-infused” (LI) catheters. FIG. 8B shows the quantification of Fg deposition on UM-catheter material (black bars) and LIS-catheter materials (white bars) by IF staining. 3 replicates with n=2-3 each. FIG. 8C shows binding of pathogens to unmodified (UM) catheters (black bars) and liquid-infused (LI) silicone (LIS) catheters (white bars). The experiment was conducted for 3 replicates with 3 samples for each replicate. The error bars represent SEM.

Based on LIS's success in reducing Fg deposition, its ability to prevent microbial surface binding was tested. Six uropathogens were grown in urine supplemented with BSA at 37° C. as shown in Table 4 below. In the table, YPD, LB, and BHI are different culture broth tunes used for growth of bacteria

TABLE 4
List of microbial strains and their corresponding growth conditions,
inoculum concentrations and antibodies.

| | Growth | Inoculum | Primary | |
|---|---|---|---|---|
| WT strain | Conditions | CFU/mL | Antibody | Reference |
| E. faecalis | 24 hr in BHI | 1 × 10\hat{ }7 | Rabbit anti- | (Flores-Mireles, |
| OG1RF | | | Streptococcus | Pinkner, Caparon, |
| | | | group D | & Hultgren, 2014) |
| | | | antigen | |
| E. coli UTI89 | 2 × 24 hr in LB | 1 × 10\hat{ }7 | Rabbit anti- | (Westfall et al., |
| HK::GFP | | | E. coli Serotype | 2019) |
| | | | O/K (ThrmoSci | |
| | | | PA1-25636) | |
| P. aeruginosa | 2 × 24 hr in LB | 1 × 10\hat{ }7 | Rabbit anti- | (Berger et al., |
| PA01 PKA | | | Pseudomonas | 2014) |
| | | | (ThermoSci | |
| | | | PA1-73116) | |
| A. baumanii | 2 × 24 hr in LB | 1 × 10\hat{ }8 | Rabbit anti- | (Di Venanzio et |
| UPAB1 | | | A. baumannii | al., 2019) |
| K. pneumoniae | 2 × 24 hr in LB | 1 × 10\hat{ }7 | Rabbit anti- | (Rosen et al., |
| TOP52 | | | K. pneumoniae | 2008) |
| | | | (ThermoSci | |
| | | | PA1-7226) | |
| C. albicans | 24 hr in YPD | 1 × 10\hat{ }7 | Rabbit anti- | (Baron et al., |
| SC3415 | | | C. albicans | 2020) |
| | | | (ThrmoSci PA1-2158) | |

Cultures were normalized in urine, added to UM control and LIS-catheters, incubated under static conditions and assessed via IF. Analysis found the LIS-catheters showed significantly reduced binding of all uropathogens when compared to UM controls (FIG. 8C). These results further demonstrate the capability of LIS-catheters to reduce not only protein deposition but to also impede microbial colonization.

### Fg Deposition and Microbial Biofilms on Catheters was Reduced by LIS.

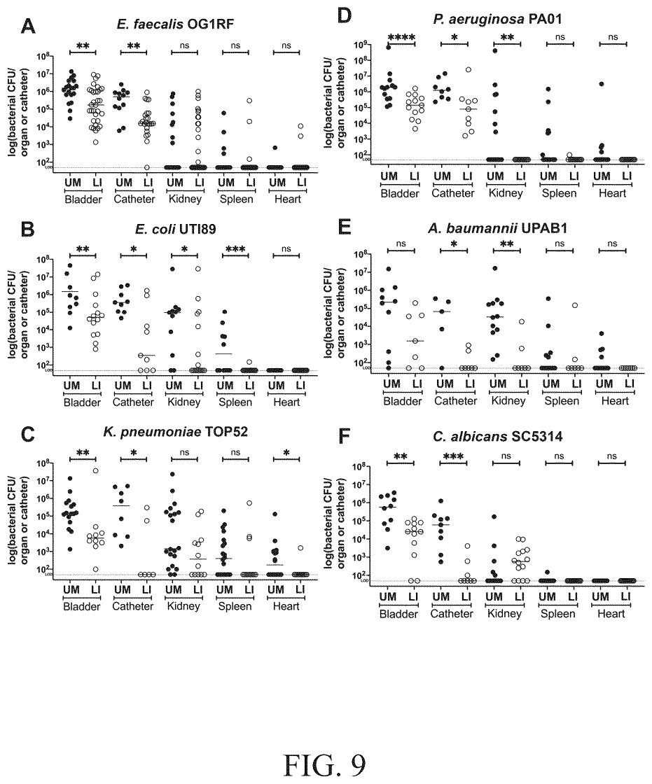

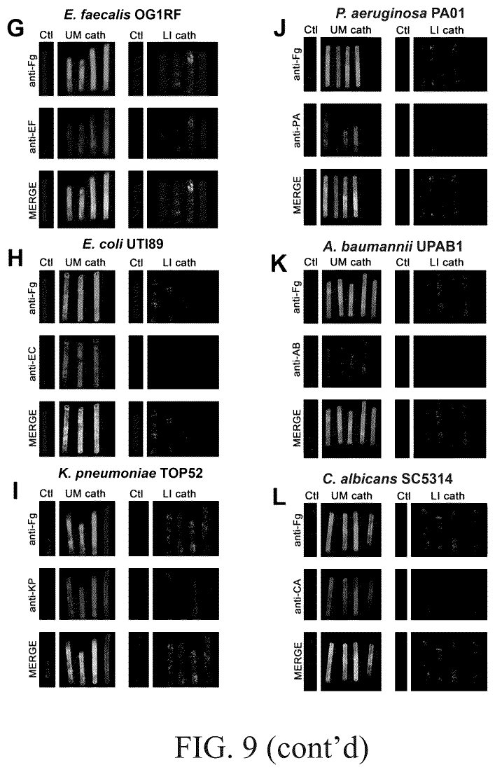

Mice were catheterized with either an UM- or LIS-catheter and challenged with ˜2×10⁷ CFU of one of six uropathogens for 24 hrs. Bladders and catheters were harvested and assessed for microbial burden by CFU (colony forming unit) enumeration or fixed for staining. Kidneys, spleens and hearts were collected to determine microbial burden. The results of the study are shown in FIG. 9.

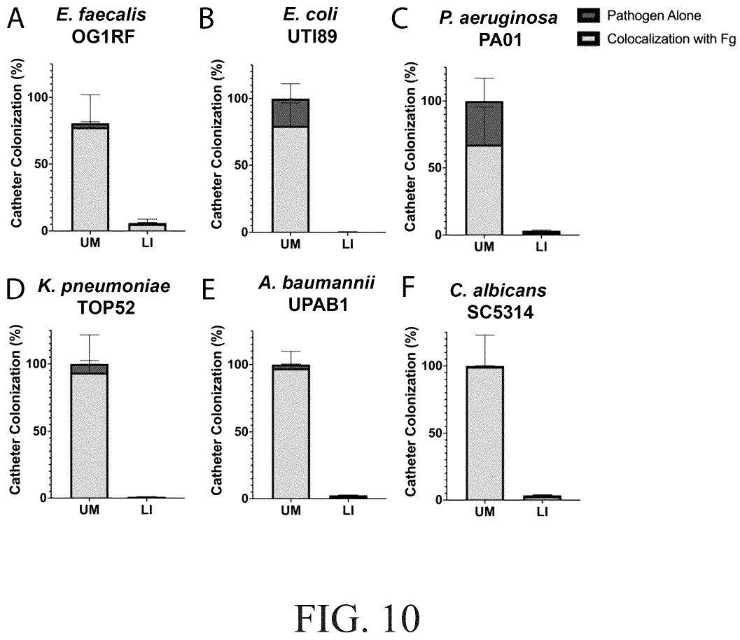

Mice with LIS-catheters had significantly reduced microbial colonization in the bladder and on catheters when compared with UM-catheterized mice regardless of the infecting uropathogen (FIG. 9, panels A-F). Organ and catheter CFUs from mice with either a UM-catheter (closed, black circles) or LIS-catheter (open, white circles) show the dissemination profile of the pathogen. FIG. 9, panels G-L show IF images of catheters. The IF images identify Fg (green), a respective uropathogen (red), and a merged image (MERGE) to compare deposition on UM-catheters (left) with LIS-catheters (right). The yellow color in the merged imaged corresponds to areas of overlap between the area of fibrinogen adhesion (green) and pathogen. Non-implanted catheters of each condition were used as controls (Ctl). All animal studies for CFUs, catheter imaging, and bladder imaging had at least 10 animals per strain and catheter type. FIG. 10, panels A-G show bar graphs representative of the quantification of uropathogen-Fg colocalization on UM and LIS-catheters from mice catheterized and infected with one of six uropathogens. 100% catheter colonization is representative of the entire area of the catheter in a given image. The height of the red bar is indicative of the relative percentage of the total area the pathogen covers to the area of the catheter in the IF image. The height of the yellow bar is the relative percentage of the area of the catheter coated with both fibrinogen (Fg) and the pathogen. Quantification was done using pixel color counter from Fiji where colocalization (yellow) of Fg (green) and pathogen (red) were quantified and compared to the total pathogen colonization of the catheter.

Additionally, colonization was significantly lower in LIS-catheterized mouse kidneys for P. aeruginosa, A. baumannii, and E. coli infections (FIG. 9, panels B, D, and E) and LI-catheterized mice infected with E. coli or C. albicans showed significantly less colonization of the spleen (FIG. 9, panels B and F). K. pneumoniae (FIG. 9, panel C) kidney and spleen colonization was not statistically significant, however they showed a trend of less colonization and significantly less colonization of the heart.

Furthermore, IF imaging and quantification of catheters confirmed decreased Fg deposition and microbial biofilms on LIS-catheters compared to UM (e.g., as shown in FIG. 10). In FIG. 10, panels A-G show quantification of uropathogen-Fg colocalization on UM and LIS-catheters from mice catheterized and infected with one of six uropathogens. Quantification was done using pixel color counter from Fiji where colocalization (yellow) of Fg (green) and pathogen (red) were quantified and compared to the total pathogen colonization of the catheter.

These data demonstrate pathogens preferentially bind to Fg, and that the LIS-modification successfully reduced Fg deposition. Fg served as the microbes' binding platform. The reduced Fg deposition disrupts uropathogen biofilm formation on catheters and colonization of the bladder in vivo.

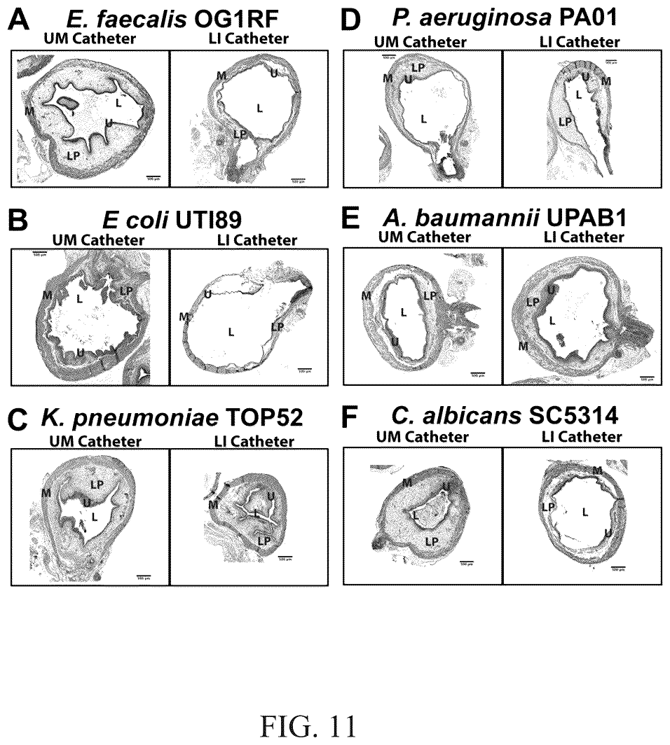

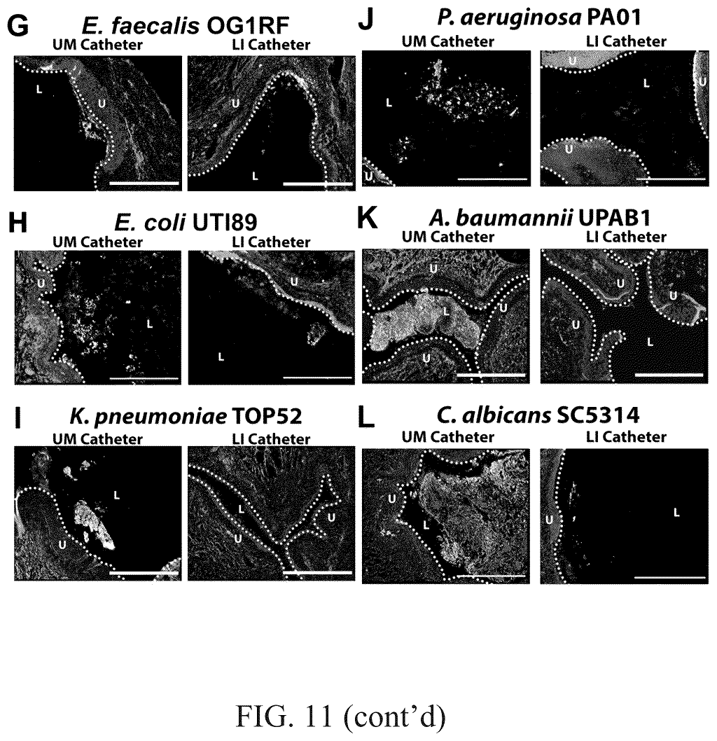

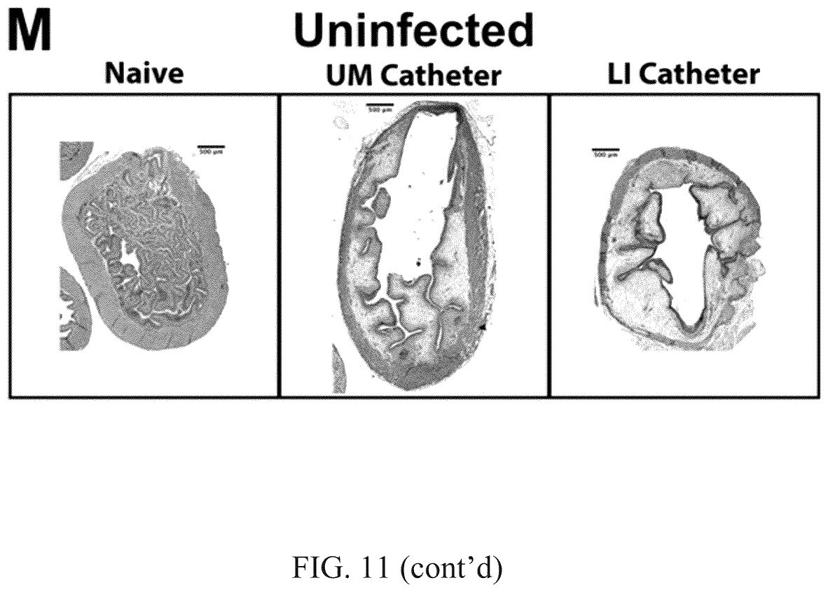

FIG. 11 shows bladders catheterized with an UM or LIS-catheters (designated as LI catheter in the images). The bladders were either infected with one of the six uropathogens described herein (panels A-F of FIG. 11 and panels G-L of FIG. 11) or were uninfected controls panel M of FIG. 11). IF images are shown in panels G-L of FIG. 11. Hematoxylin and eosin (H&E) stained bladders are shown in panels A-F and M of FIG. 11. Bladders were stained for nuclei (blue), Fg (green), respective uropathogens (red), and neutrophils (white). Yellow arrows on the IF images indicate microbes in LIS-catheterized bladders. The urothelial/lumen boundaries are outlined in white dotted lines and labeled U (urothelium) and L (lumen). All scale bars are 500 μm.

Hematoxylin and eosin (H&E) analysis shows the LIS-catheter does not exacerbate bladder inflammation regardless of the presence of infection or not (panels A-F and M of FIG. 11), an important factor to account for when developing a new medical device. For some pathogens, the LIS-catheter results in less inflammation than bladders catheterized with an UM-catheter (FIG. 11, panels A, B, and F). Furthermore, Fg presence, uropathogen colonization, and neutrophil recruitment was examined in UM- and LIS-catheterized and infected bladders by IF microscopy as shown in panels G-L of FIG. 11. This analysis revealed a reduction of microbial colonization as well as decreased neutrophil recruitment in the LIS-catheterized mice as compared to UM-catherized mice.

Importantly, H&E analysis (FIG. 11, panels A-F and M) shows the LIS-catheter does not exacerbate bladder inflammation regardless of the presence of infection or not, which is an important factor to account for when developing a new medical device. In fact, for some pathogens, the LIS-catheter results in less inflammation than bladders catheterized with an UM-catheter (FIG. 11, panels A, B, and F). Furthermore, the inventors examined Fg presence, uropathogen colonization, and neutrophil recruitment in UM- and LIS-catheterized and infected bladders by IF microscopy (FIG. 11, panels G-L). This analysis revealed a reduction of microbial colonization as well as decreased neutrophil recruitment in LIS-catheterized mice as compared to UM-catheterized mice.

LIS Modification Reduces Protein Deposition on Catheters in CA UTI Mouse Model of E. faecalis.

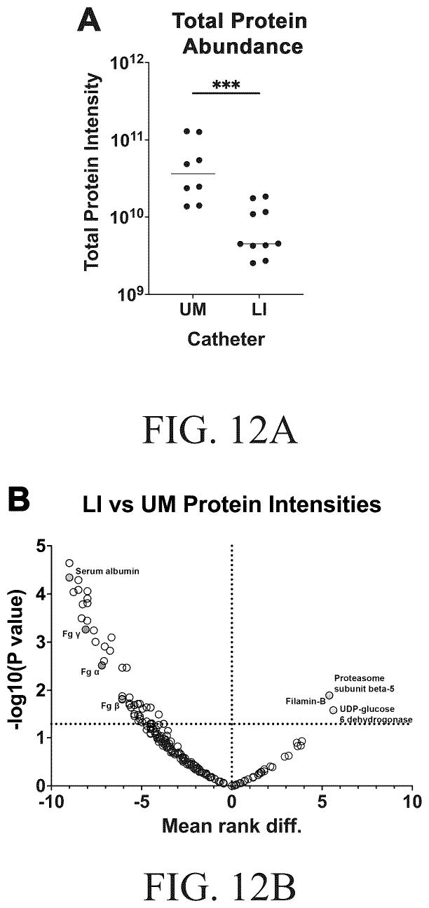

A quantitative-proteomics comparison was performed to identify proteins deposited on UM- and LIS-catheters retrieved 24 hpi with E. faecalis. Harvested catheters were prepared and protease digested with trypsin as in Zougman et al. (2014). nLC-MS/MS was performed in technical duplicate and label-free-proteomics (LFQ) processed as in Cox and Mann, 865 proteins were identified at a 1% FDR (Cox & Mann, 2008). The total abundance of protein was significantly reduced in LIS-catheters vs UM-catheters (FIG. 12A). A Mann-Whitney test was used to evaluate significance (p=0.0005). Additionally, abundance of Fg and over 130 other proteins significantly decreased while only three proteins showed a significant increase (UDP-glucose 6-dehydrogenase, filamin-B, and proteasome subunit beta type-5) (FIG. 12B). This data further demonstrates that the LIS modification not only reduced Fg deposition but also a wide variety of host-proteins, which could play a role in microbial colonization and biofilm formation as demonstrated earlier with BSA (see e.g., FIG. 5).

### LIS-Catheter Reduces Host-Protein Deposition In Vivo

A subset of UM-catheters and LIS-catheters taken from mice 24 hpi (hours post-infection) with E. faecalis were assessed for protein deposition via mass spectrometry as shown in FIGS. 12A and 12B. 4 UM (unmodified) catheters and 5 LI (liquid infused) silicone (LIS)-catheters were used. In the figures, LI represents liquid infused silicone catheters. UM represents unmodified catheters. In FIG. 12A, intensities of the 95% most abundant proteins were summed using a total proteome approach and compared between the UM-catheter and the LIS-catheter groups. In FIG. 12B, a volcano plot was created for a subset of proteins using the mean rank difference and Mann-Whitney statistical analysis to generate p-values. Negative mean rank difference indicates less protein on the LIS-catheter than on the UM-catheter and a significant difference is shown with a −log₁₀(P-value) over 1.3. The Fg chains (α-, β-, and γ-) are highlighted in green, serum albumin in orange, UDP-glucose 6-dehydrogenase, filamin-B and proteasome subunit beta type-5 are in yellow.

### Image Montage of Liquid Infused and Unmodified Catheters

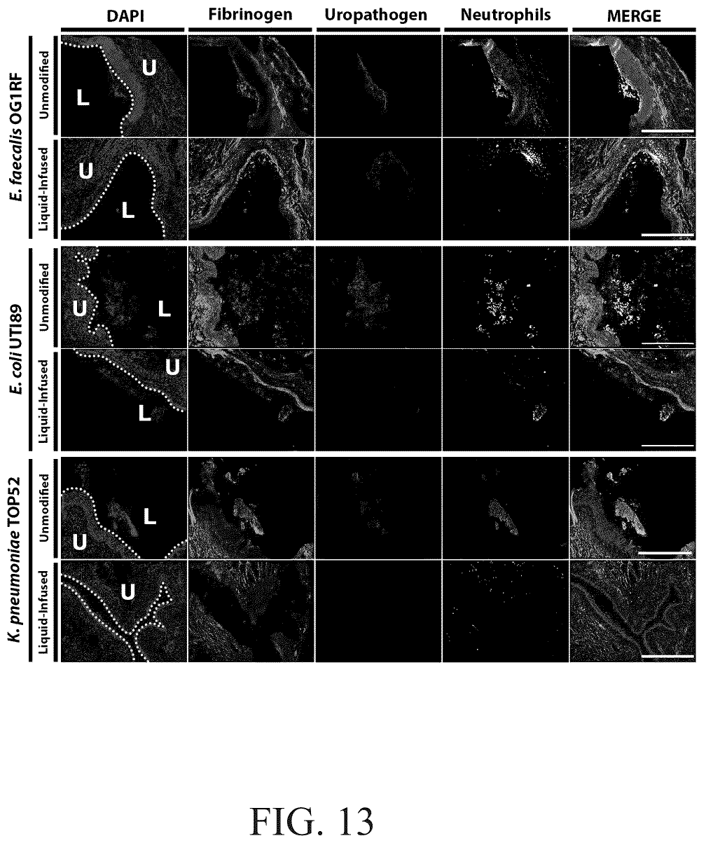

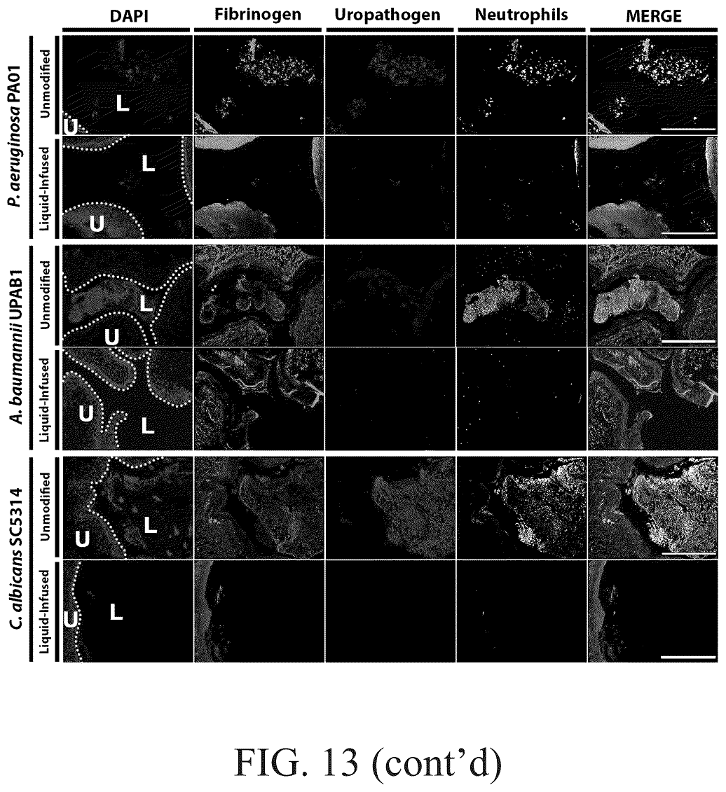

FIG. 13 is an image montage of liquid infused silicone and unmodified catheters. Mice were implanted with either an unmodified catheter (UM) or a liquid infused silicone (LIS) catheter and infected with 1×10⁶ CFU of the respective uropathogens labeled at the end of each row. At 24 hpi, bladder tissues were harvested, fixed, and parafilm-embedded. The bladder tissues were then subjected to IF analysis. Antibody staining was used to detect Fg (anti-Fg; green), uropathogens (red), neutrophils (anti-Ly6G; white) and cell nuclei (DAPI; blue). Scale bars are 500 μm. Images are stitched 2×2 tiles taken at 20× magnification.

### DISCUSSION

To the inventors' knowledge, this is the first study to show a diverse set of uropathogens including gram-negative, gram-positive, and fungal species interact with Fg to more effectively bind to silicone urinary catheter surfaces. Furthermore, disrupting Fg deposition with LIS-catheters reduced the ability of uropathogens to bind and colonize the catheter surface and bladder in an in vivo CAUTI mouse model. Moreover, LIS also reduced dissemination of E. coli, P. aeruginosa, and A. baumannii into the kidneys and other organs. Finally, LIS-catheters did not increase inflammation and, for half of the pathogens, inflammation was reduced. Furthermore, the deposition of other host-secreted proteins on LIS-catheters is around 6.5 fold less then UM-catheters. Together, these findings indicate that catheters made using LIS are a promising antibiotic sparing approach for reducing or preventing CAUTIs by interfering with protein deposition.

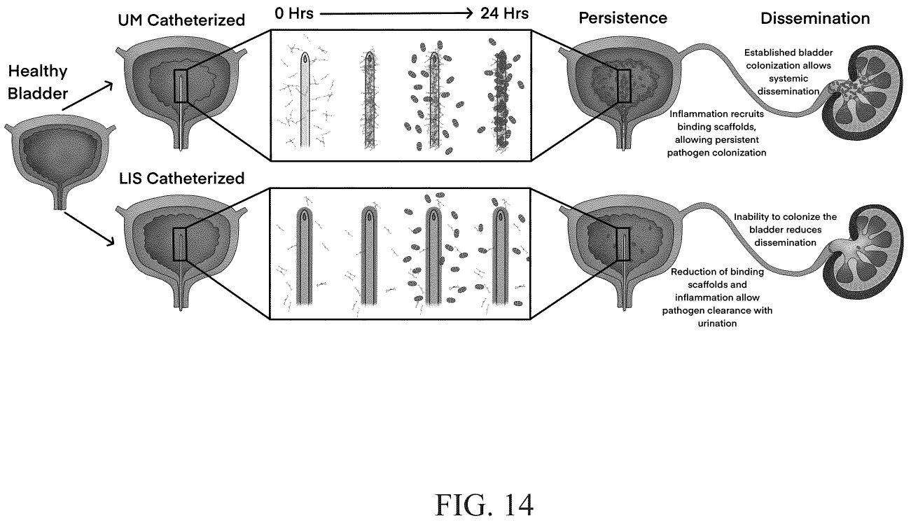

FIG. 14 is an illustrative embodiment of liquid-infused silicone (LIS)-catheter reducing bladder inflammation, incidence of catheter-associated urinary tract infection (CAUTI), and dissemination. Urinary catheter-induced inflammation promotes the release of Fg into the bladder to heal physical damage. Consequently, this Fg is deposited onto the catheter creating a scaffold for incoming pathogens to bind, establish infection, and promote systemic dissemination. However, catheterization with a LIS-catheter reduces Fg deposition onto its surface; thus, reducing the availability of a binding scaffolds for incoming pathogens. Consequently, overall bladder colonization and systemic dissemination are reduced making LIS-catheters a strong candidate for CAUTI prevention.

Pathogen-Fg interaction is important during urinary catheterization for both E. faecalis and S. aureus. Binding to Fg is critical for efficient bladder colonization and biofilm formation on the catheter via protein-protein interaction using EbpA and CHB adhesins, respectively and their disruption hinders colonization. Gram-negative pathogens, A. baumannii and P. mirabilis co-localize with Fg during urinary catheterization; however, the bacterial factors and any mode of interaction have not been described, to inventors' knowledge. Interaction of E. coli and K. pneumoniae with Fg during CAUTI has not been described.

Type 1 pili, a chaperon-usher pathway (CUP) pili, allows pathogens to colonize the bladder urothelium by binding to mannosylated receptors on the urothelial surface through the tip adhesin FimH. Furthermore, other CUP pili including the P pili, important for pyelonephritis, and the Fml pilus, important for colonizing inflamed bladder urothelium, bind specifically to sugar residues Galα1-4Gal in glycolipids and Gal(β1-3)GalNAc in glycoproteins, respectively. Interestingly, Fg is highly glycosylated, containing a wide variety of sugar residues including mannose, N-acetyl glucosamine, fucose, galactose, and N-acetylneuraminic acid. Without wishing to be bound to any particular theory, the glycosylation in Fg may be recognized by CUP pili for colonization. Furthermore, A. baumannii CUP1 and CUP2 pili are essential for CAUTI, this together with its interactions with Fg in vivo, suggests that these pili may play a role in Fg interaction. Similarly, P. aeruginosa also encodes CUP pili, CupA, CupB, CupC and CupD, which are important for biofilm formation. Furthermore, C. albicans has several adhesins, ALS1, ALS3, and ALS9, which have a conserved peptide binding cavity shown to bind to Fg γ-chain (Hoyer & Cota, 2016).

Inhibition of initial uropathogen binding reduces colonization and biofilm formation on urinary catheter surfaces and prevent subsequent CAUTI. To prevent surface binding, a variety of modified surfaces impregnated with antimicrobial or bacteriostatic compounds have been generated and, have proven to reduce microbial binding in vitro but not in vivo. Without wishing to be bound to any particular theory, in vitro studies do not efficiently mimic the complexities of the in vivo environment, for example; 1) Growth media: the majority of the in vitro studies use laboratory rich or defined culture media, and laboratory media does not recapitulate the catheterized bladder environment that pathogens encounter. Specifically, urine culture conditions activate different bacterial transcriptional profiles than when grown in defined media, which may affect microbial persistence and survival. 2) Host factors: host-secreted proteins are released into the bladder due to catheter-induced physical damage and subsequent inflammation. Without wishing to be bound to any particular theory, these proteins are deposited on the catheter surface and can hinder the release of antimicrobials or block interaction of antimicrobials with the pathogen. As has been observed with Fg deposition, host-protein deposition is not uniform, which may lead to antimicrobial release or pathogen-antimicrobial agent interaction at a sub-inhibitory concentrations. Consequently, these interactions can contribute to the development of multidrug-resistance among uropathogens.

Based on the role of deposited host-proteins in promoting microbial colonization, antifouling catheter coatings present a better approach to decreasing CAUTI prevalence rather than using biocidal or biostatic compounds, such as antibiotics, that promote resistance. Antifouling coatings are made from polymers and have shown resistance to protein deposition; however, these coatings can become unstable over time and be difficult to produce. However, antifouling coatings made from polymers antifouling coating can be challenging to produce in large quantities. Furthermore, molecular degradation and desorption can affect the integrity and function of the hydration shells over time.

Most reports on the use of purely antifouling coatings to combat CAUTI have shown successful reduction in bacterial colonization in vitro, but not in vivo. Many antifouling coatings are optimized to target bacterial adhesion, as it is understood that the first stage of biofilm development is bacterial attachment to a surface. However, in a complex environment such as the in vivo bladder, the first change to the catheter surface is the adhesion of a complex set of host-generated proteins and biological molecules, generally referred to as a conditioning film, which can mask the surface. Yet studies on catheter coatings to-date have rarely focused on the role of the host in infection establishment; namely, the host-secreted proteins. Data presented herein suggest that this missing element may at least partly explain the differing results seen for most antifouling catheter treatments in vitro vs in vivo.

This example used a clinically relevant silicone oil to create a simple liquid-infused polymer that was not only bacteria-resistant but also protein-resistant, filling in the missing link between in vitro and in vivo work, the conditioning film. Furthermore, lack of an exacerbated inflammation response seen in the results shows reduced capsule formation in implants in which the liquid layer has been mechanically removed or stripped from the surface of the silicone substrate, suggesting that the use of a substrate infused with a silicone oil and not having a free overlayer of silicone oil conveys additional anti-inflammatory benefits.

A deeper understanding of the pathogenesis of CAUTI is critical to moving beyond current developmental roadblocks and create more efficient intervention strategies. Infusion of silicone with an immiscible liquid and resulting in a coating that does not form an overlayer significantly decreases Fg deposition and microbial binding as shown herein by using in vitro conditions that more thoroughly recapitulate the catheterized bladder environment. Importantly, in vitro results presented herein were confirmed in vivo using a mouse model of CAUTI as described herein. Data presented herein shows that LIS-catheters are refractory to bacterial colonization without targeting microbial survival, which often leads to antimicrobial resistance, and thus holds tremendous potential for the development of lasting and effective CAUTI treatments. These types of technologies are needed to achieve better public health by decreasing healthcare-associated infections and promoting long-term wellness.

### Materials and Methods:

### Mouse Infection Models

Mice used in this study were ˜6-week-old female wild-type C57BL/6 mice purchased from Jackson Laboratory and The National Institute of Cancer Research. Mice were subjected to transurethral implantation and inoculated as previously described (Conover, Flores-Mireles, Hibbing, Dodson, & Hultgren, 2015). Briefly, mice were anesthetized by inhalation of isoflurane and implanted with a 6-mm-long UM-silicone or LIS-catheter. Mice were infected immediately following catheter implantation with 50 μl of ˜2×10⁷ CFU/mL in PBS introduced into the bladder lumen by transurethral inoculation (unless otherwise noted (ST1). For all mouse experiments microbes were grown in their corresponding media (ST1). To harvest the catheters and organs, mice were sacrificed at 24 hrs post infection by cervical dislocation after anesthesia inhalation; the silicone catheter, bladder, kidneys, heart and spleen were aseptically harvested. Catheters were either subjected to sonication (Branson, Ultrasonic Bath) for CFU enumeration analysis, fixed for imaging via standard IF procedure described above, or sent for proteomic analysis as described using nonimplanted catheters as controls. Bladders for immunofluorescence and histology were fixed and processed as described below. Kidneys, Spleens and Hearts were all used for CFU analysis. The University of Notre Dame Institutional Animal Care and Use Committee approved all mouse infections and procedures as part of protocol number 18-08-4792MD and #22-016971. All animal care was consistent with the Guide for the Care and Use of Laboratory Animals from the National Research Council.

### Bladder IHC and H&E Staining of Mouse Bladders

Mouse bladders were fixed in formalin overnight, before being processed for sectioning and staining as described in Walker et al., 2017. Briefly, bladder sections were deparaffinized, rehydrated, and rinsed with water. Antigen retrieval was accomplished by boiling the samples in Na-citrate, washing in water, and then incubating in PBS three times. Sections were then blocked (1×PBS, 1.5% BSA, 0.1% Sodium Azide), washed in PBS, and incubated with appropriate primary antibodies overnight at 4° C. Next, sections were washed with PBS, incubated with secondary antibodies for 2 h at room temperature (RT), and washed once more in PBS prior to Hoechst dye staining. Hematoxylin and Eosin (H&E) stain for light microscopy was done by the CORE facilities at the University of Notre Dame (ND CORE). All imaging was done using a Zeiss inverted light microscope (Carl Zeiss, Axio Observer). Zen Pro (Carl Zeiss, Thornwood, NY) and ImageJ software were used to analyze the images.

### Human Urine Collection

Human urine was collected and pooled from at least two healthy female donors between 20-40 years of age. Donors had no history of kidney disease, diabetes or recent antibiotic treatment. Urine was sterilized using a 0.22 μm filter (Sigma Aldrich) and pH 6.0-6.5. When supplemented with Bovine Serum Albumin (BSA) (VWR Lifesciences), urine was filter sterilized again following BSA addition. All participants signed an informed consent form and protocols were approved by the local Internal Review Board at the University of Notre Dame under study #19-04-5273.

### Microbial Growth Conditions in Supplemented Urine

E. faecalis, and C. albicans were grown static for ˜5 hrs in 5 mL of respective media (Table 2) followed by static overnight culture in human urine supplemented with 20 mg/mL BSA (urine BSA20). E. coli, K. pneumoniae, P. mirabilis, A. baumanii and P. aeruginosa were grown 5 hrs shaking at 37° C. in LB then static for 24 hours, supplemented into fresh urine BSA for another 24 hours static (2×24 hrs) in urine BSA20. All cultures were washed in PBS (Sigma) 3 times and resuspended in assay appropriate media.

### Silicone Disk Preparation

8 mm disks of UM-silicone (Nalgene 50 silicone tubing, Brand Products) or LIS were cut using a leather hole punch. UM disks were washed 3 times in PBS and air dried. LIS disks were stored in filter sterilized modifying liquid at RT. Disks were skewered onto needles (BD) to hold them in place and put in 5 mL glass tubes (Thermo Scientific) or placed on the bottom of 96 well plate wells (Fisher Scientific) (UM silicone only). Plates and glass tubes were UV sterilized for >30 min prior to use.

### Protein Binding Assays

Human fibrinogen free from plasminogen and von Willebrand factor (Enzyme Research Laboratory #FB3) was diluted to 150 ug/mL in PBS. 500 uL of 150 ug/mL Fg was added to each disk in glass tubes, sealed, and left over night at 4° C. Disks were then processed according to standard immunofluorescence (IF) procedure as described in Colomer-Winter et al., 2019. Briefly, disks were washed 3 times in PBS, fixed with 10% Neutralized formalin (Leica), blocked, and stained using Goat anti-Fg primary antibody (Sigma) (1:1000) and Donkey anti-Goat IRD800 secondary antibody (Invitrogen) (1:1000). Disks were then dried over night at 4° C. and imaged on an Odyssey Imaging System (LI-COR Biosciences) to examine the infrared signal. Intensities for each catheter piece were normalized against a negative control and then made relative to the pieces coated with Fg which was assigned to 100%. Images were processed using Image Studio Software (LI-COR, Lincoln, NE) Microsoft Excel and graphed on GraphPad Prism (GraphPad Software, San Diego, CA).

### Microbial Binding Assays

For assessing the effect of protein deposition on microbial binding, 100 uL of 150 ug/mL Human Fg, 100 μL of 150 μg/mL BSA, or 100 μL of PBS were incubated on UM-silicone disks in 96 well plates overnight at 4° C. The following day disks were washed 3 times with PBS followed by a 2 hr RT incubation in 100 uL of urine containing microbes at a concentration of ˜10\hat{ }8 CFU/mL. For assessing microbial binding to UM-silicone versus LIS, 500 uL of microbe containing media was added to prepared disks in glass tubes. Standard IF procedure was then followed as described herein using goat anti-Fg and rabbit anti-microbe primary antibodies (1:1000) (see ST1 for details). Secondary antibodies used were Donkey anti-Goat IRD800 and Donkey anti-Rabbit IRD680 (1:5000). Quantification of binding was done using ImageStudio Software (LI-COR). Intensities for each catheter piece were normalized against a negative control and then made relative to the pieces coated with Fg which was assigned to 100%.

### Weight Measurement of Silicone Tubes and Tygon® Tubes Versus Infusion Time

Five samples of 20 cm Tygon® tube (14-171-219, Saint-Gobain Tygon S3™ 3603 Flexible Tubings, Fisher Scientific, USA) or silicone tube (8060-0030, Nalgene™ 50 Platinum-cured Silicone Tubing, Thermo Scientific, USA) were utilized in weight measurement. Weight of the tubes prior to infusion were measured with an analytical balance (AL204, Analytical Balance, Mettler Toledo, Germany), results were marked as “0 h infused in silicone oil”. After the measurement of the initial weights, the tubes were incubated with silicone oil (DMS-T15, Polydimethylsiloxane, trimethylsiloxy, 50 cSt, GelestSInc., USA) until designated time points. For each time point, tubes were removed from the oil with forceps and held vertically for 30 seconds for the excess silicone oil to flow out of the tube. The bottoms of the tubes were then gently dabbed with Kimwipes™ (Kimwipe, Kimberly-Clark Corp., USA), and then subjected to weight measurement. After measurement, the tubes were placed back into silicone oil until the next time point. Tubes were measured every 3 hrs for the first 2 days; every 6 hrs from day 3 to day 6; and every 24 hrs from day 6 and onwards. Measurements were taken until data showed no significant increase, and that the plateau trendline consist of at least 3 data points.

### Weight Measurement of Mouse Catheters Versus Infusion Time

Five samples of 20 cm mouse catheter (SIL 025, RenaSil Silicone Rubber Tubing, Braintree Scientific, Inc., USA) were utilized in weight measurement. Weight of the tubes prior to infusion were measured with an analytical balance, results were marked as “0 min infused in silicone oil”. After the measurement of the initial weights, the tubes were incubated with silicone oil until designated time points. For each time point, catheters were removed from the oil with forceps, a Kimwipes™ was immediately pressed against the bottom of the catheters to remove the excess silicone oil via capillary action. After the excess oil was drained, catheters were then subjected to weight measurement. The catheters were placed back into silicone oil until the next time point. Catheters were measured every 1 minute for the first 5 minutes of the experiment; every 2 minutes from 5-15 minutes; and every 5 minutes from 15 minutes and onwards. Measurements were taken until data showed no significant increase, and that the plateau trendline consist of at least 3 data points.

### Parameter Measurement of Silicone Tube Before and After Infusion

The length, inner diameter and outer diameter of the silicone tubes were measured before silicone oil infusion and after complete infusion (after incubating with silicone oil for >7 days). All parameters were measured using a digital caliper (06-664-16, Fisherbrand™ Traceable™ Digital Calipers, Fisher Scientific, USA).

### Parameter Measurement of Mouse Catheters Before and After Infusion

The length, inner diameter, and outer diameter of the mouse catheters were measured before silicone oil infusion and after complete infusion (after incubating with silicone oil for >30 minutes). The length of the mouse catheter was measured using a digital caliper. Photos of the tube openings of the catheters and a scale of known length were taken. The inner and outer diameter were then estimated via ImageJ. Percentage weight change of Tygon® tube, silicone tube and mouse catheters were calculated based on the formula below:

### Proteomic Analysis of Mouse Catheters

Five mice were catheterized with a LIS-catheter, 4 mice were catheterized with an UM-catheter and catheters harvested 24 hrs after infection with E. faecalis OG1RF. Harvested catheters were put into 100 μL of SDS buffer (100 mM Tris HCl pH-8.8, 10 mM DTT and 2% SDS), then vortexed for 30 sec, heated for 5 min at 90° C., sonicated for 30 min and the process repeated once more. Samples were sent to the Mass Spectrometry and Proteomics Facility at Notre Dame (MSPF) for proteomic analysis. Proteins were further reduced in DTT, alkylated and digested with trypsin using Suspension Trap and protocols (Zougman, Selby, & Banks, 2014) nLC-MS-MS/MS was performed essentially as described in (Sanchez et al., 2020) on a Q-Exactive instrument (Thermo).

Proteins were identified and quantified using MaxLFQ (Label Free Quantification) within MaxQuant and cutoff at a 1% FDR (Cox & Mann, 2008). This generated a total of 8 data records from UM-catheters and 10 from LIS-catheters. Data reduction was performed by removing contaminants proteins. Protein abundance for each catheter type was of 105 then calculated by summing the LFQ intensity of proteins which comprised 95% of the total abundance on the catheters. Strict filtering criteria of at least 2 replicates with technical replication from the UM-catheters and 3 replicates with technical duplication from the LIS-catheters were required to keep an identification. Abundance of the reduced proteins was plotted using Graph Pad Prism. Statistical significance was tested using a Mann-Whitney U test. A volcano plot was created using the ranked mean difference for each protein and −log of calculated P-values with an alpha=0.05.

### Statistical Analysis

Unless otherwise stated, in the current example, data from at least 3 experiments were pooled for each assay. Significance of experimental results were assessed by Mann-Whitney U test using GraphPad Prism, version 7.03 (GraphPad Software, San Diego, CA). Significance values on graphs are *p≤0.05, **p≤0.01, ***p≤0.001 and ****p≤0.00001.

Antibodies Used in this Study

Primary antibodies against microbial pathogens used in the study are listed in Table 2. Primary antibodies against non-pathogens used are provided as follows: Fg, Goat anti-Fibrinogen and neutrophils, Rat anti-Ly6G.

Secondary antibodies used for IF in the study: IRDye 800CW donkey anti goat (LI-COR) and IRDye 680LT Donkey anti-rabbit (LI-COR). Secondary antibodies for IHC; Donkey anti-goat (Life Technologies Corporation), Donkey anti-rabbit (Invitrogen), Donkey anti-mouse (Invitrogen) and Donkey anti-rat (Invitrogen).

### VII. Experimental Example 2

Described herein is an embodiment of a method of modifying catheters. In certain embodiments, the methods and devices described significantly reduce deposition of Fg on the surface, while allowing for adhesion of non-Fg proteins.

### Catheter Modification Protocol

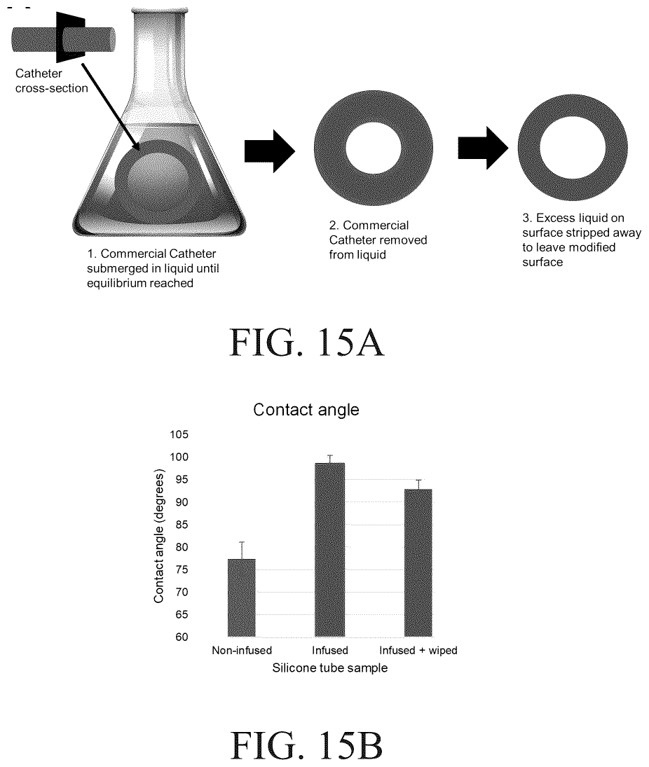

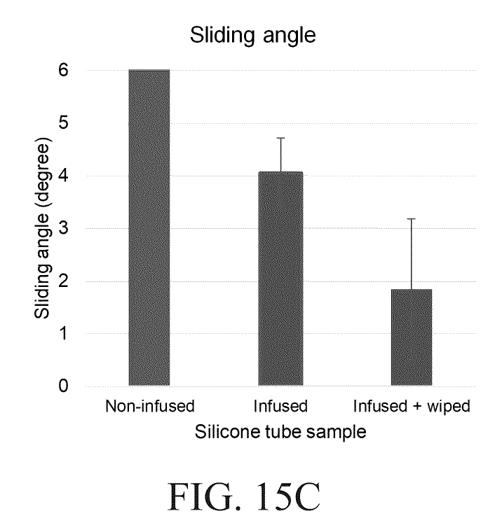

Inventors developed a surface treatment that significantly reduces deposition of Fg on silicone catheters in vivo while significantly increasing the adhesion of non-Fg proteins. The coatings are created on commercially available catheters by submerging the entire catheter in a free silicone liquid with the capacity to diffuse throughout the polymer until equilibrium is reached as shown in FIG. 15A. For example, in an embodiment, a commercial silicone catheter is immersed in a silicone oil of trimethoxy-terminated polydimethylsiloxane fluid. Then, excess liquid on the catheter surface is removed via physical stripping of the layer (e.g., mechanical stripping), leaving behind a physically and chemically altered surface which changes the way in which proteins interact with the surface.

FIG. 15B is a graph showing the contact angle of a non-infused (red), an infused sample with an overlayer of silicone oil (purple outlined in red), and a silicone oil infused sample which was wiped (purple). The results show that silicone oil infused and wiped samples (purple) have a lower contact angle than infused samples (purple outlined in red) which have not been wiped and have an overlayer of impregnation fluid on them as depicted in FIG. 15A. However, the contact angle is higher than non-infused silicone.

FIG. 15C is a graph showing the sliding angle of a non-infused (red), an infused sample with an overlayer of silicone oil (purple outlined in red), and an infused sample which was wiped (purple). The sliding angle of the infused+wiped sample (purple) is much lower than either the infused or non-infused samples.

### Mechanism for Altered Adhesion

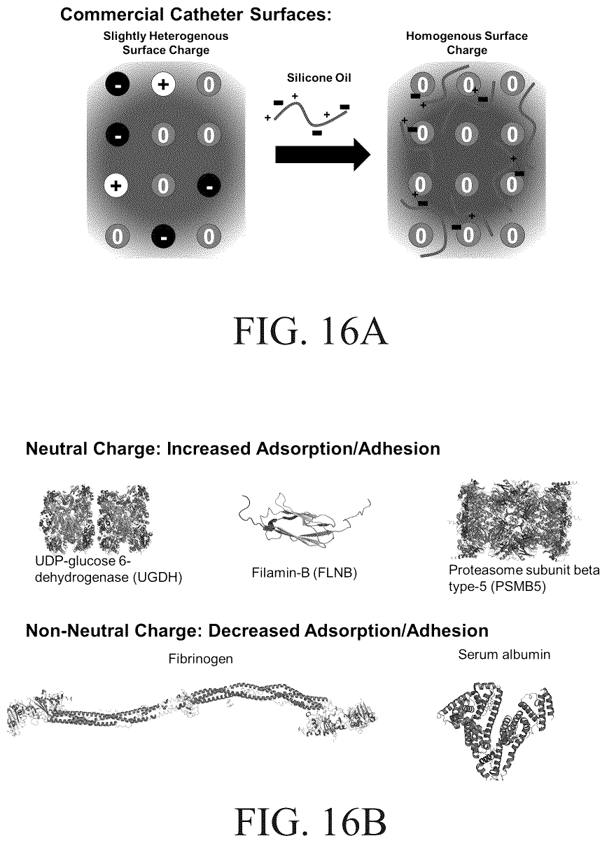

Without wishing to be bound to any particular theory, the surface of polydimethylsiloxane (PDMS) presents a locally heterogeneously charged surface to which proteins such as fibrinogen (Fg) and serum albumin adsorb. The semi-ionic nature of siloxane molecules is caused by difference in electronegativity between the silicone and oxygen atoms. Introducing unbound siloxane molecules into the system (e.g., via a silicone oil) allows the new compound liquid/solid material to reach charge equilibrium, as the free molecules are drawn via attraction forces to regions where their own ionic charges cancel out those of the cross-linked solid polymer as shown in FIG. 16A.

FIG. 16A shows an illustrative embodiment of the mechanism of action. Commercial silicone catheters have nm-scale heterogeneity of surface charge. Adding a free silicone fluid, which can migrate through the polymer and associate positive charges to negative (and vise-versa), allows the system to become closer to fully neutral. In this way, proteins with weaker surface charges, such as UGDH, FLNB, and PSMB5 are more likely to interact with the surface than proteins with locally positive or negative surface charges such as Fg and serum albumin, which instead are more likely be drawn to other charged molecules within the complex in vivo environment. These proteins are depicted in FIG. 16B. FIG. 16B lists examples of proteins, which show both increased and decreased adhesion after infusion.

The use of free siloxane molecules, which can migrate dynamically within the polymer bulk, allows for the treatment of any commercial silicone mixture, regardless of specific composition, as the free molecules will naturally adapt to the specific charge distribution created by the use of different additives, cross-linkers, or curing protocols.

### VIII. Experimental Example 3

Among other things, described herein are changes of silicone substrates under different infusion conditions.

FIG. 2 is an illustration of the different infusion conditions used in the experiment. In the experiment, silicone samples were immersed for a period of time to infuse silicone samples with a silicone oil impregnation fluid. Weight measurements were taken over a period of 80 hours in order to determine the amount of silicone oil absorbed by the sample over time.

For fully impregnated samples, the silicone samples were immersed in silicone oil and removed from the silicone oil after the samples were substantially fully impregnated with silicone oil. The free liquid overlayer of silicone oil was not removed from the surface of the samples, as is shown in the figure.

For the overlayer stripped samples, the silicone samples were immersed in silicone oil and removed from the silicone oil after the samples were substantially fully impregnated (“fully infused”) with silicone oil. The free liquid overlayer of silicone oil was removed mechanically from the surface of each of the samples as shown in FIG. 2.

% Qₘₐₓ refers to various degrees of partial impregnation of silicone substrates. The silicone samples were immersed in silicone oil for varying periods of time and removed from the silicone oil after the sample was infused with silicone oil at the desired % Qₘₐₓ.

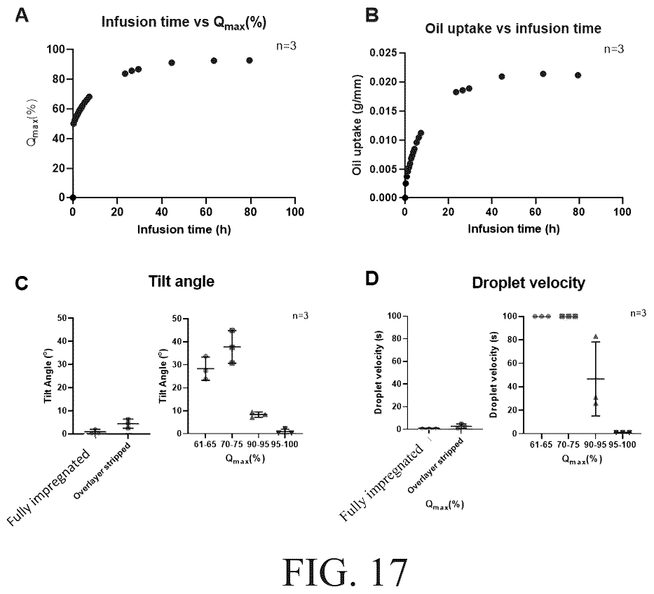

FIG. 17, panels A-D show changes of silicone samples under different infusion conditions. % Qₘₐₓ refers to various degrees of partial impregnation of silicone substrates. In FIG. 17, panel A, % Qₘₐₓ increases with increasing infusion time, then reaches plateau after 60 hours of infusion. FIG. 17, panel B shows oil uptake by silicone samples increases with increasing infusion time, then plateaus after 60 hours of infusion. FIG. 17, panel C shows the tilt angle of silicone samples infused under different infusion conditions. FIG. 17, panel D shows droplet velocity of silicone samples infused under different infusion conditions. Samples without droplet movement are marked as 100 s in the graph. For all graphs, error bars show the standard deviation (SD), n=3.

Among other things, these results demonstrate that the two different tested methods of removing the excess free overlayer of silicone oil (i.e., overlayer stripping and partial infusion) are fundamentally different from the “fully infused” samples with a free overlayer of silicone oil.

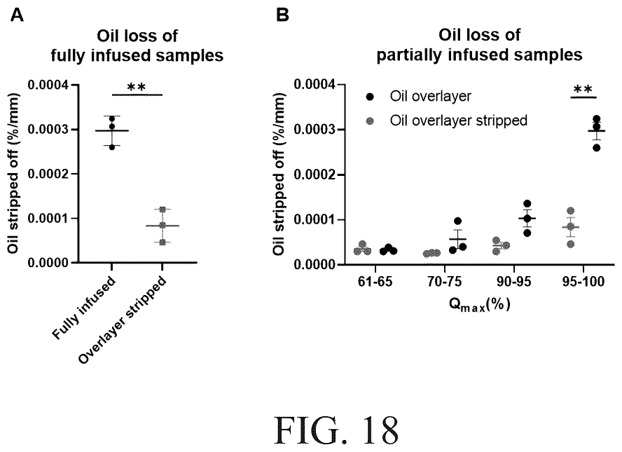

Continuing with the experimental examples, FIG. 18, panels A and B show silicone oil loss of silicone samples via repeated exposure of air-water interface. In FIG. 18, the overlayer stripped samples have a % Qₘₐₓ from 95% to 100%. In FIG. 18, the partially impregnated samples have a % Qₘₐₓ from 90% to 94%. FIG. 18, panel A shows silicone oil loss of fully impregnated silicone samples with or without the free silicone oil overlayer stripped. FIG. 18, panel B shows partially impregnated silicone samples with or without the free silicone oil overlayer stripped. These silicone samples were then tested for silicone oil loss through water dipping. For all graphs, error bars show the standard deviation (SD). Differences between groups were tested for significance using the Welch's t test. **, P<0.005. n=3.

These results demonstrate that the two different methods of removing the excess overlayer (i.e., overlayer stripping and partial impregnation) do significantly reduce the amount of free silicone liquid that can be physically removed from the surface. For example, after mechanically stripping the overlayer, fully or partially impregnated silicone samples experience less loss of oil than samples which have not been stripped of silicone oil.

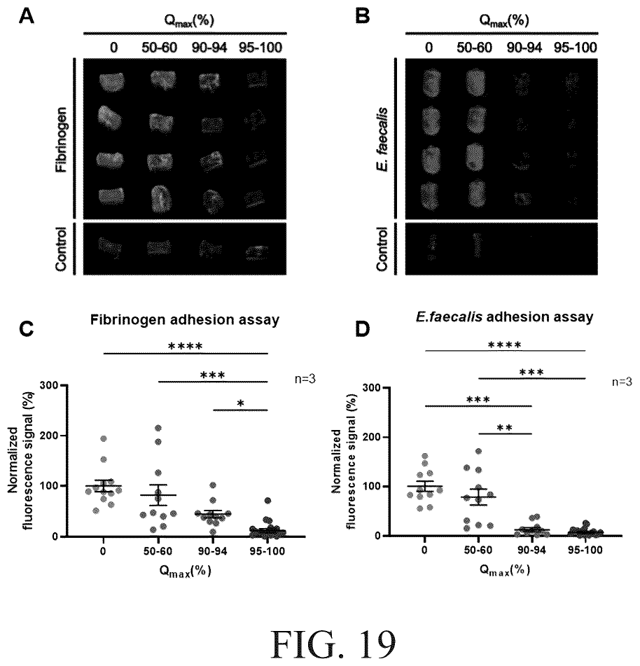

FIG. 19, panels A-D show fibrinogen (Fg) and E. faecalis adhesion levels decrease with increasing degrees of impregnation of the substrate. FIG. 19, panel A shows silicone discs were stained with immunofluorescence (IF) for Fg deposition (green). FIG. 19, panel B shows silicone discs were stained with IF for E. faecalis (red) binding. Non-infused silicone discs incubated in PBS were used as controls as controls for autofluorescence. FIG. 19, panel C shows quantification of Fg localization on silicone discs from panel A. FIG. 19, panel D shows quantification of E. faecalis localization on silicone discs from panel B. For all graphs, error bars show the standard deviation (SD). Differences between groups were tested for significance using the Kruskal-Wallis test. *, P<0.05; **, P<0.005; ***, P<0.0005 and ****, P<0.0001. 3 replicates of n=3-4 each were performed for each condition.

These results demonstrate that the two different methods of removing excess free silicone overlayer (i.e., overlayer stripping and partial impregnation) result in a significant reduction of the amount of protein and E. faecalis bacteria adhesion as compared to controls that did not undergo infusion with silicone oil.

### IX. Experimental Example 4

Among other things, the present example shows differences between PDMS catheters which have not been infused with silicone oil, PDMS catheters which have been infused with silicone oil (LIS-catheters), and LIS-catheters that have had the overlayer stripped.

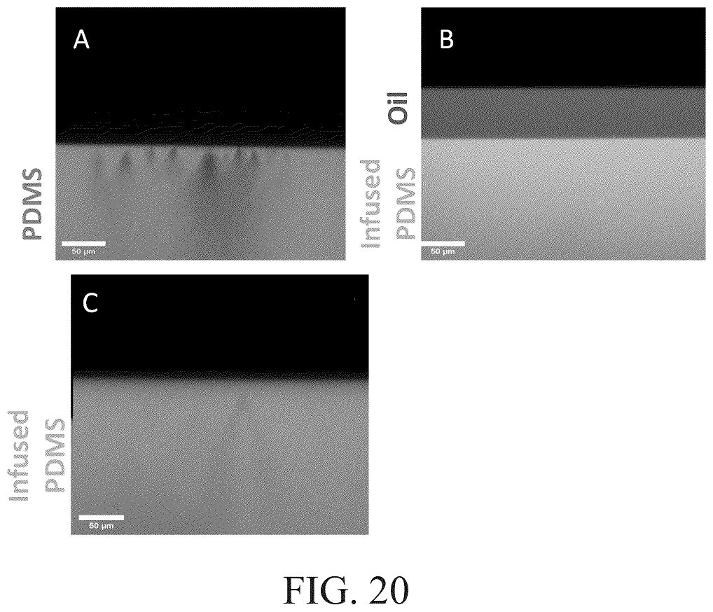

Overlayer stripping removes free silicone oil from the surface of PDMS substrates, which is shown in FIG. 20. Overlayer stripping was achieved by infusing a catheter segment with silicone oil until equilibrium was reached, draining excess silicone oil from the catheter, then rolling the catheter on an absorbent tissue (Kimwipe, Kimberly-Clark Corp., USA) to remove any free silicone liquid from the surface. FIG. 20, panel A shows a confocal microscopy image of a PDMS substrate that has not been infused with any silicone oil. The green shading in the image shows the PDMS. FIG. 20, panel B shows a confocal microscopy image of a PDMS substrate which has an overlayer of silicone oil. The overlayer of silicone oil is shown in red. The yellow shading of the infused PDMS indicates the infusion of silicone oil (red) into the PDMS substrate (green). FIG. 20, panel C shows the effect of overlayer stripping on an infused PDMS substrate. There is no silicone oil (red) on the surface of the infused PDMS (yellow) as shown in the figure. The stripping process removes the overlayer of silicone oil from the infused PDMS. The scale bar for the images is 50 μm.

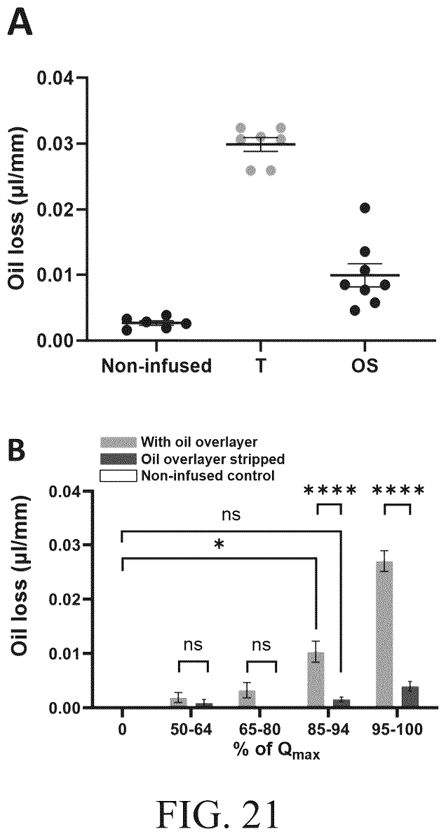

FIG. 21, panels A and B demonstrate that LIS-catheters with free liquid removed (i.e., by overlayer stripping) show a reduced loss of free liquid into an environment as compared to fully infused LIS-catheters. PDMS catheter segments were prepared by infusing the segment with dyed silicone oil. To prepare the infusion solution, 9 mg of pyrromethene (05971, Pyrromethene 597-8C9, Exciton, USA) was added to every 100 ml of silicone oil and mixed thoroughly. The solution was then filtered through a 0.45 μm filter to remove any particulates. Samples were then infused and, where needed, stripped of their excess liquid overlayer as previously described. In brief, overlayer stripping was achieved by infusing catheter segments with silicone oil until equilibrium was reached. Excess silicone oil was drained from the catheter, then rolled on an absorbent tissue (Kimwipe, Kimberly-Clark Corp., USA) to remove any free silicone liquid from the surface of the catheter.

Once prepared, any excess dyed surface oil was removed by passing the samples through an air/water interface of a 10 mL volume of DI water 10 times in succession. A 1 ml aliquot of toluene (108-883, Toluene anhydrous, Alfa Aesar, USA) was then added to the DI water containing the removed silicone oil, manually shaken for 1 minute, then left to settle for at least 1 minute to allow the tolune and water phases to separate. The top layer was carefully extracted and placed in a glass cuvette for spectrophotometer (840-277000, GENESYS™ 30 Visible Spectrophotometer, Thermo Fisher, USA) measurement. The absorbance of the samples was measured at 2 nm intervals within the 350-650 nm range, with the presence of a peak indicating the presence of dyed silicone oil that had been removed from the catheter segment. All results were standardized to the size of the sample catheter segment in mm and reported as either μL of oil or the percentage of total amount of oil infused into the sample oil lost to the DI water per mm of catheter length.

FIG. 21, panel A shows the amount of silicone oil removed from a non-infused catheter section as discussed above. The panel shows amounts of silicone oil removed from fully infused LIS-catheter sections (T) and fully infused LIS-catheter sections with the silicone oil overlayer stripped (OS). In FIG. 21, panel A, more silicone oil is removed from the fully infused LIS-catheter than the fully infused LIS-catheter where the overlayer has been stripped. The “*” and “****” symbols are indicative of the level of significance. For FIG. 21, panel A, “*” indicates P<0.05 and “****” indicates P<0.0001.

FIG. 21, panel B shows the amount of silicone oil removed from LIS-catheter sections at varying levels of infusion with silicone oil either having the overlayer stripped (dark grey) or left intact (light grey). More silicone oil was removed from LIS-catheters that did not have the overlayer stripped than from LIS-catheters that did have the overlayer stripped. LIS-catheters were grouped at the following levels of infusion as measured by Qₘₐₓ: 50%-64%, 65-80%, 95-94%, and 95-100%. LIS-catheters with a Qₘₐₓ of 95-100% are deemed to be fully infused. A negative control was also provided which has a Qₘₐₓ of 0%. The catheter had not been infused at all with silicone oil. At each level of infusion, the silicone oil overlayer was either stripped (overlayer stripping, OS) or left intact (traditional infusion, T). The statistical significance of the experimental results was evaluated using Mann-Whitney U Tests performed in GraphPad Prism, version 7.03 (GraphPad Software, San Diego, CA). Significance levels on the graphs are denoted as follows: *p≤0.05, **p≤0.01, ***p≤0.0001, and ****p≤0.00001.

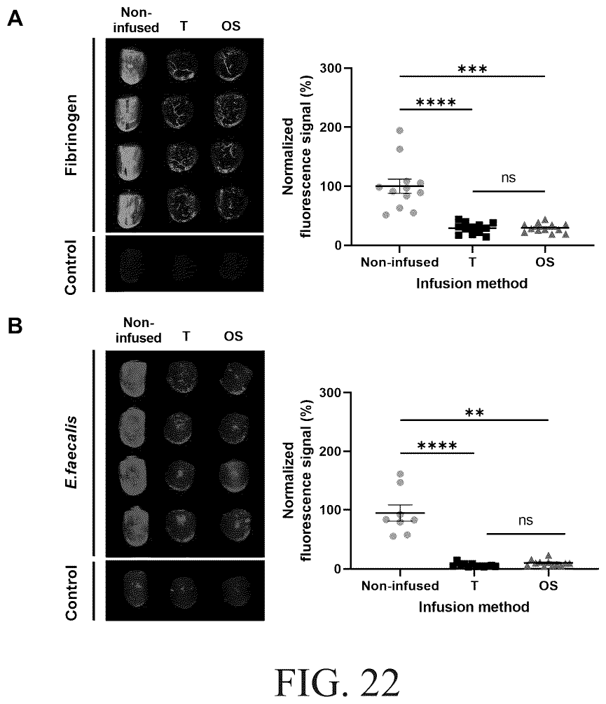

FIG. 22, panels A and B demonstrate that LIS-catheters with free silicone oil stripped from the surface of the catheter remain functional. FIG. 22, panels A and B characterize protein and bacterial adhesion on LIS-catheter sections. PDMS catheters were not infused (“non-infused”) with silicone oil, fully infused with silicone oil (traditional infusion, T), or fully infused with silicone oil and had the overlayer of silicone oil stripped (OS).

FIG. 22, panel A shows fibrinogen adhesion images (left) and quantification (right) on non-infused (control) PDMS catheters, fully infused LIS-catheters (traditional infusion without overlayer stripping, T), and fully infused LIS-catheters with the overlayer stripped (OS). Catheters used for this experiment were PDMS catheters infused with silicone oil. As shown in FIG. 22, panel A, fully infused LIS-catheters with the overlayer stripped had a reduced amount of fibrinogen adhering to them as compared to LIS-catheters that had not been infused with silicone oil.

FIG. 22, panel B shows E. faecalis adhesion images (left) and quantification (right) on non-infused (control) PDMS catheters, fully infused LIS-catheters (traditional infusion without overlayer stripping, T), and fully infused LIS-catheters with the overlayer stripped (OS). As shown in FIG. 22, panel B, fully infused LIS-catheters with the overlayer stripped had a reduced amount of E. faecalis adhering to them as compared to PDMS catheters that have not been infused with silicone oil.

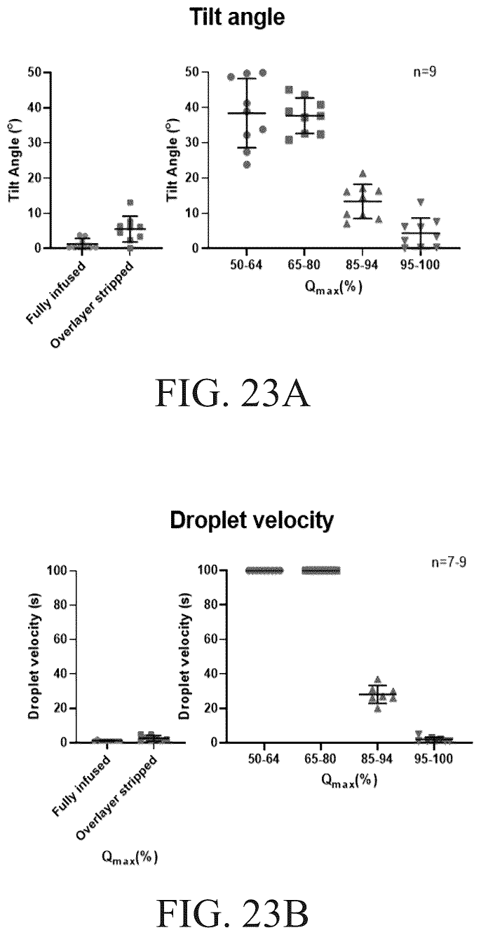

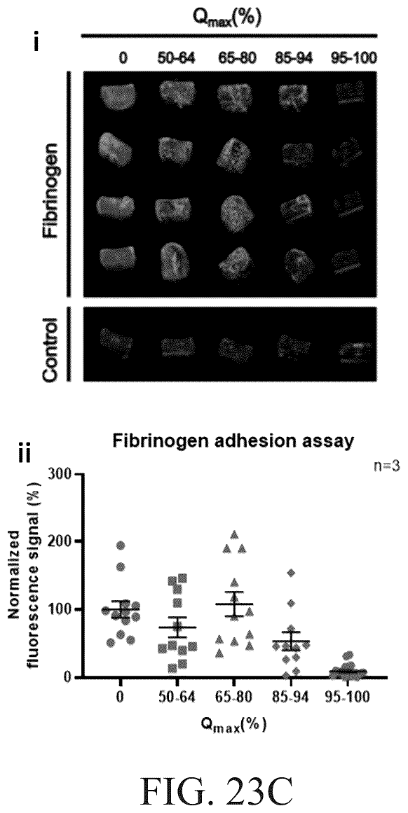

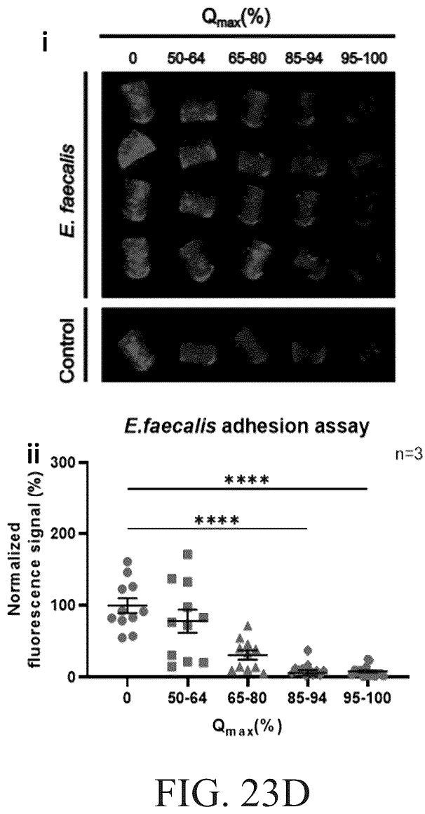

FIGS. 23A-23D further show that LIS-catheters with free silicone oil overlayer removed continue to retain a surface on which water droplets will not stick. A surface on which water droplets will not stick will have a low tilt angle (degrees °) and low (i.e., fast) droplet velocity (s), whereas a surface on which water will stick will have a high tilt angle and a high (i.e., slow) droplet velocity.

FIG. 23A shows tilt angle analysis of LIS-catheters. In the left panel of FIG. 23A, the tilt angle of a fully infused LIS-catheter is shown and compared to a fully infused LIS-catheter in which the silicone oil overlayer has been stripped. In the right panel of FIG. 23A, the tilt angle of LIS-catheters with both the free liquid overlayer intact (fully infused) and with the overlayer stripped are shown at complete infusion (left plot) and at different levels of infusion (right plot). LIS-catheters in this panel have had the overlayer of silicone oil stripped from the surface of the LIS-catheter. As the level of infusion increases, there is a corresponding decrease in tilt angle.

FIG. 23B shows droplet velocity analysis of LIS-catheters. In the left panel of FIG. 23B, the droplet velocity of a fully infused LIS-catheter is shown and compared to a fully infused LIS-catheter in which the silicone oil overlayer has been stripped. In the right panel of FIG. 23B, the droplet velocity of LIS-catheters is shown at different levels of infusion. LIS-catheters in this panel have had the overlayer of silicone oil stripped from the surface of the LIS-catheter. At higher levels of infusion, the droplet velocity decreases (i.e., the droplet moves faster). The maximum droplet velocity recorded for experiments was 100 s, which represents the upper time limit for this experimental measurement.

Fully infused LIS-catheter sections with the overlayer stripped were comparable in both tilt angle and droplet velocity to a fully infused LIS-catheter section with an intact overlayer as shown in FIGS. 23A and 23B. Additionally, decreasing functionality was observed with a decreasing Qmax of (e.g., amount of silicone oil infused in) the LIS-catheters.

FIG. 23C, panels i and ii show fibrinogen adhesion to LIS-catheters which have had the overlayer stripped. FIG. 23C, panel i shows a series of fluorescence images of fibrinogen (green) adhering to LIS-catheters at different levels of silicone oil infusion. FIG. 23C, panel ii shows, quantitatively, the amount of fibrinogen adhering to LIS-catheters with the overlayer stripped. As the amount of silicone oil infused into the LIS-catheters decreases (i.e., as Qmax decreases), there in an increase in the amount of fibrinogen adhering to the LIS-catheters.

FIG. 23D, panels i and ii show E. faecalis adhesion to LIS-catheters which have had the overlayer stripped. FIG. 23D, panel i shows a series of fluorescence images of E. faecalis (red) adhering to LIS-catheters at different levels of infusion. FIG. 23D, panel ii shows, quantitatively, the amount of E. faecalis adhering to LIS-catheters with the overlayer stripped. As the amount of silicone oil infused into the LIS-catheters decreases (i.e., as Qmax decreases), there in an increase in the amount of E. faecalis adhering to the LIS-catheters.

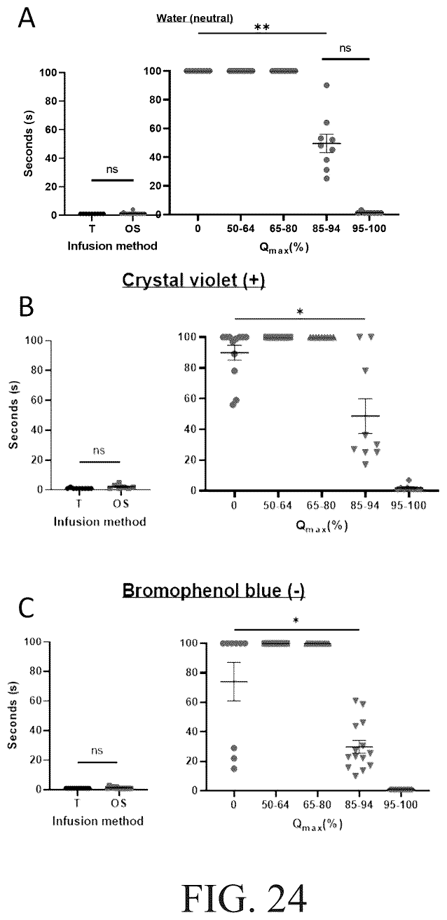

FIG. 24, panels A-C show LIS-catheters with the free liquid overlayer stripped 4 from the surface respond differently to differently charged liquids. Without wishing to be bound to a particular theory, the results suggest that charge plays a role in the functionality of LIS-catheters. FIG. 24, panels A-C show droplet sliding velocity for water droplets with a neutral (FIG. 24, panel A), positive (FIG. 24, panel B), or negative charge (FIG. 24, panel C). The time indicated on the graph is the time it takes for the droplet to travel a 2-cm length of the sample. For the purposes of the present experiment, water was used a neutrally charged liquid, water with crystal violet was used as a positively charged liquid, and water with bromophenol blue was used as a negatively charged liquid.

The protocol for measuring droplet sliding velocity is described as follows. A section of catheter tubing being tested (2 cm, length of 8060-0030, Nalgene™ 50 Platinum-cured Silicone Tubing, Thermo Scientific, USA) was placed on a tilt stage at 30°. A digital angle gauge (AccuMASTER 2 in 1 Magnetic Digital Level and Angle Finder, Calculated Industries) was affixed to the tilt stage to ensure the angle is maintained. A 20 μl droplet containing water, crystal violet, or bromophenol blue was introduced into the tubing's lumen using a pipette. The time taken for the droplet to travel from the beginning to the end of the tube was measured. To ensure precise time measurement, a camera was utilized to record the entire experiment for a frame-by-frame analysis.

The results in the panels show a difference in average sliding speed for non-infused PMDS substrates (i.e., substrates with a 0 Qmax) for both positively charged and negatively charged droplets as compared to the neutrally charged droplets. At the lowest level of infusion (i.e., 50%-64% Qmax), the difference in sliding speed between positively charged droplets, negatively charged droplets, and neutrally disappears, which suggests that charge neutralization is playing a role in the functionality of LIS-catheters.

### X. Experimental Example 5

Among other things, the present example shows differences between liquid infused substrate (LIS) PDMS catheters (LIS-catheters) as compared to PDMS catheters that were unmodified (UM). The silicone oil overlayer of the LIS-catheters were stripped in this experiment as the catheters were inserted into mice bladders. LIS-catheters described in this example have been infused with a silicone oil. In particular, the present example concerns differences in E. coli catheter-associated urinary tract infections (UTIs) and systemic dissemination during prolonged urinary catheterization.

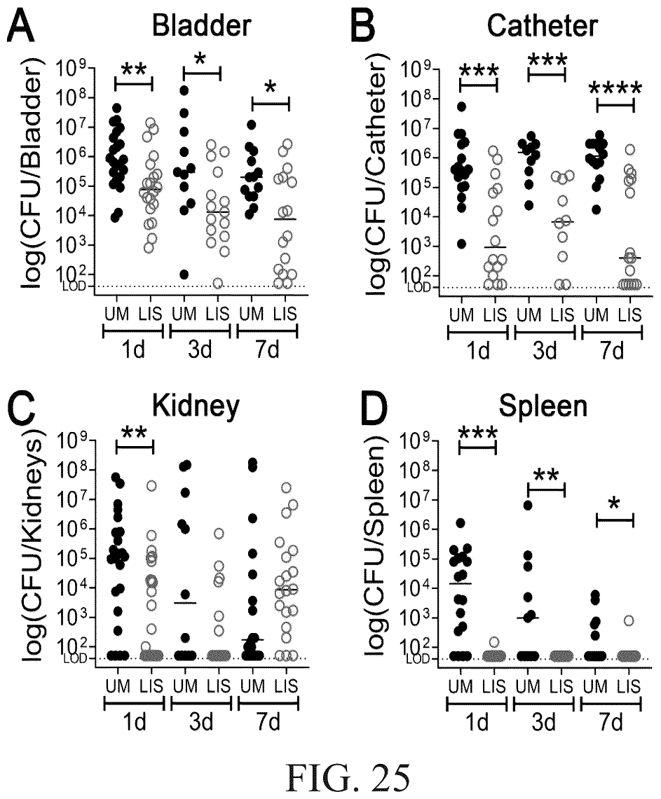

The inventors tested the ability of liquid infused substrate catheters (LIS-catheters) to reduce uropathogenic E. coli colonization in bladders as compared to PDMS catheters that were unmodified. The systemic dissemination of E. coli at 1, 3, and 7 days post infection and catheterization was tested in animal models. For this experiment, mice were catheterized with either an UM- or LIS-catheter and challenged with ˜2×10⁷ CFU of uropathogenic E. coli. An hour before catheterizing the mice, the LIS-catheters were removed from the silicon oil solution, the remaining silicon oil was drained, and catheters were let to dry. Furthermore, if any remaining the overlayer was present that was stripped away during the passage of the LIS-catheter though the urethra. At 1, 3, and 7 days post-infection, bladders and catheters were harvested and assessed for microbial burden by CFU (colony forming unit) enumeration or fixed for staining. Kidneys, spleens and hearts were collected to determine microbial burden. FIG. 25, panels A-D show LIS-catheters reduced uropathogenic E. coli catheter-associated UTIs and systemic dissemination during prolonged urinary catheterization in mice. All animal studies for colony forming units (CFUs) had at least 10 mice per strain and catheter type. Differences between groups were tested for significance using the Mann-Whitney U test. The following levels of significance are presented in the panels: *, P<0.05; **, P≤***, P<0.0005; and ****, P<0.0001.

These data demonstrate that LIS-catheters (labeled as “LIS” in FIG. 25, panels A-D) were effective in reducing bladder and catheter colonization at 1, 3, and 7 days post infection and catheterization as compared to unmodified (UM) (i.e., not infused) PDMS catheters. In FIG. 25, panel C, LIS-catheters reduced E. coli colonization of kidneys at 1 day post infection and catheterization. In FIG. 25, panel D, colonization of E. coli in the spleen was reduced at all-time points.

Elements of different implementations described herein may be combined to form other implementations not specifically set forth above. Elements may be left out of the processes and devices described herein without adversely affecting their operation. Various separate elements may be combined into one or more individual elements to perform the functions of devices or methods described herein.

Throughout the description, where devices or systems are described as having, including, or comprising specific components, or where processes and methods are described as having, including, or comprising specific steps, it is contemplated that, additionally, there are apparatus, and systems of the described technology that consist essentially of, or consist of, the recited components, and that there are processes and methods according to the described technology that consist essentially of, or consist of, the recited processing steps.

While the described technology has been particularly shown and described with reference to specific embodiments, it should be understood by those skilled in the art that various changes in form and detail may be made therein without departing from the spirit and scope of the described technology.

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of modifying a polymeric substrate of a medical device, the method comprising:
infusing the polymeric substrate with an impregnation fluid such that the polymeric substrate is impregnated with the impregnation fluid.

2. The method of claim 1, wherein the polymeric substrate is biocompatible.

3. The method of claim 1, wherein the polymeric substrate comprises silicone.

4. The method of claim 3, wherein the silicone comprises polydimethylsiloxane (PDMS).

5. The method of claim 1, wherein the polymeric substrate comprises a hydrogel, poly(acrylic acid), poly(vinyl alcohol), poly(vinylpyrrolidone), poly(ethylene glycol), polyacrylamide, or a polysaccharide hydrogel.

6. The method of claim 1, wherein the impregnation fluid comprises a hydrophilic liquid or a hydrophilic ionic liquid.

7. The method of claim 1, wherein the polymeric substrate comprises an organogel.

8. The method of claim 7, wherein the organogel comprises one or more of the following materials: anthracene, anthraquinone and steroid-based molecules.

9. The method of claim 1, wherein the impregnation fluid is silicone oil.

10. (canceled)

11. The method of claim 9, wherein the viscosity of the silicone oil is from 0.65 cSt to 10,000 cSt.

12.-13. (canceled)

14. The method of claim 1, wherein the impregnation fluid comprises one or more of the following: low-volatility polydimethylsiloxanes, cyclic polydimethylsiloxanes, silicone emulsions, silicone fluid blends, thermal silicone fluids, alkyl silicones, aryl-alkyl silicones, fluorosilicone fluids, hydrophilic silicones, polar silicones, ampliphilic silicones, low-temperature fluids, and naturally derived silicones.

15. The method of claim 9, wherein the method comprises removing substantially all of the free silicone oil from a surface the polymeric substrate.

16. The method of claim 15, wherein the method comprises mechanically or chemically removing post-infusion excess silicone oil from the surface of the polymeric substrate.

17. The method of claim 9, wherein the method does not produce an immobilized liquid layer of silicone on the surface of the polymeric substrate.

18. (canceled)

19. The method of claim 9, wherein infusing the polymeric substrate with the silicone oil comprises impregnating the polymeric substrate from 50% to 99.99% of the maximum absorption capacity of the polymeric substrate.

20-21. (canceled)

22. The method of claim 1, wherein the method reduces adhesion and/or adsorption of one or more protein(s) to a surface of the polymeric substrate.

23. The method of claim 22, wherein the one or more protein(s) comprise fibrinogen or serum albumin.

24. The method of claim 1, wherein the method increases adhesion and/or adsorption of one or more protein(s) to the polymeric substrate.

25. The method of claim 24, wherein the one or more protein(s) comprises one or more members of the group consisting of: UDP-glucose 6-dehydrogenase (UGDH), filamin B (FLNB), and Proteasome 20S Subunit Beta 5 (PSMB5).

26. (canceled)

27. The method of claim 1, wherein the polymeric substrate comprises an outward facing surface which interfaces with a tissue.

28. The method of claim 1, wherein the polymeric substrate comprises an inward facing surface which interfaces with a biological fluid.

29.-30. (canceled)

31. A method of modifying a urinary catheter to alter protein adhesion to a polymeric substrate of the catheter, the method comprising:
infusing, by immersion for a period of time, the polymeric substrate of the urinary catheter with a silicone oil, wherein the polymeric substrate comprises silicone, wherein the period of time is characterized in that the polymeric substrate is impregnated with the silicone oil; and
removing substantially all of the silicone oil from the surface(s) of the polymeric substrate such that the surface(s) of the polymeric substrate does not comprise free silicone oil.

32. (canceled)

33. A medical device comprising:
a polymeric substrate, wherein the polymeric substrate is infused with an impregnation fluid.

34.-59. (canceled)
